(19) United States
(12) Patent Application Publication
(10) Pub. No.: US 2025/0270529 A1
(43) Pub. Date: Aug. 28, 2025

(54) ENGINEERED CRISPR-CAS13F SYSTEM AND USES THEREOF

(71) Applicant: HuidaGene Therapeutics (Singapore) Pte. Ltd., Singapore (SG)

(72) Inventors: Huawei Tong, Shanghai (CN); Xing Wang, Shanghai (CN)

(21) Appl. No.: 18/696,184

(22) PCT Filed: Sep. 29, 2022
(86) PCT No.: PCT/CN2022/122833

§ 371 Date: Mar. 27, 2024

(30) Foreign Application Priority Data
Sep. 29, 2021 (WO) PCT/CN2021/121926
Mar. 28, 2022 (WO) PCT/CN2022/083461

(51) Int. Cl. C12N 9/22; C12N 15/11; C12N 15/86; C12N 15/90

(52) U.S. Cl. CPC C12N 9/226; C12N 15/11; C12N 15/86; C12N 15/907; C12N 2310/20; C12N 2750/14143

(57) ABSTRACT

The disclosure provides novel engineered Cas13f effector proteins that substantially maintain guide sequence-specific cleavage activity and substantially lack guide sequence-independent collateral cleavage activity and uses thereof, such as in RNA-based target gene transcript knock down.

### CROSS-REFERENCE TO RELATED APPLICATIONS

This application is a U.S. national stage application filed under 35 U.S.C. § 371 based on International Patent Application No. PCT/CN2022/122833, filed on Sep. 29, 2022, which claims the benefit of and priority to International Patent Application No. PCT/CN2021/121926, filed on Sep. 29, 2021, entitled “Engineered CRISPR-Cas13 System and Uses Thereof”, and International Patent Application No. PCT/CN2022/083461, filed on Mar. 28, 2022, entitled “Engineered CRISPR-Cas13 System and Uses Thereof”. The entire contents of each of the aforementioned applications, including any sequence listing and drawings, are incorporated herein by reference in their entireties.

### REFERENCE TO AN ELECTRONIC SEQUENCE LISTING

The contents of the electronic sequence listing (“HGP020PCT.xml”; Size is 17,778 bytes and it was created on Sep. 29, 2022) is incorporated herein by reference in its entirety.

### TECHNICAL FIELD

The subject matter disclosed herein is generally directed to systems, methods, and compositions used for targeted RNA modification and editing utilizing systems comprising engineered Cas13f polypeptides. In particular, the present disclosure provides RNA-targeting compositions comprising novel engineered Cas13f polypeptides and at least one targeting nucleic acid component.

### BACKGROUND OF THE DISCLOSURE

CRISPR-Cas13 is quickly becoming a widely adopted RNA editing technology. This system can use its sequence specific guide RNA to selectively modify (e.g., cut or cleave via endonuclease activity) a target RNA, such as mRNA. Compared to the permanent genomic changes introduced by DNA-based editing, RNA controls gene expression at the transcription level, thus providing a safer and more controllable gene therapy approach. Because of the high RNA editing efficiency of the CRISPR-Cas13 systems, they have already been widely used in a number of organisms including yeast, plant, mammal, and zebra fish (see (Abudayyeh et al., 2017; Aman et al., 2018; Cox et al., 2017; Jing et al., 2018; Konermann et al., 2018). An ortholog of CRISPR-Cas13d, CasRx, could mediate RNA knockdown in vivo and effectively alleviate disease phenotypes in various mouse models (He et al., Protein Cell 11:518-524, 2020; Zhou et al., Cell 181:590-603 e516, 2020; and Zhou et al., National Science Review 7:835-837, 2020).

One drawback from these currently identified Cas13 proteins, however, is that they all have non-specific/collateral RNase activity upon activation by crRNA-based target sequence recognition. This activity is particularly strong in Cas13a and Cas13b, and still detectably exists in Cas13d and, to a lesser extent, in Cas13e, for example. While this property can be advantageously used in nucleic acid detection methods, the non-specific/collateral RNase activity of these Cas13 proteins also causes undesirable collateral degradation of bystander RNAs, and has imposed a major barrier for their in vivo application, such as in gene therapy.

On the other hand, for practical utilities such as SHERLOCK that relies on collateral activity for sensitive detection, it can be beneficial to have mutant Cas13f effector proteins that exhibit even higher collateral activity compared to wild type Cas13f.

Thus, there is a need to further optimize wild type Cas13 in the art for different purposes, e.g., either to lower collateral cleavage activity with acceptable on-target cleavage activity for certain uses such as therapeutical applications, or to enhance/increase collateral cleavage activity with acceptable on-target cleavage activity for certain other uses such as diagnostic applications.

Citation or identification of any document in this application is not an admission that such a document is available as prior art to the disclosure.

### SUMMARY

One aspect of the disclosure provides an engineered Cas13f polypeptide, wherein the engineered Cas13f polypeptide:
(1) comprises a mutation in a region spatially close to a) the N-terminal endonuclease catalytic RXXXXH motif (e.g., the N-terminal endonuclease catalytic RNFYSH motif) of a reference Cas13f polypeptide (e.g., of SEQ ID NO: 1), and/or b) the C-terminal endonuclease catalytic RXXXXH motif (e.g., the C-terminal endonuclease catalytic RNKALH motif) of the reference Cas13f polypeptide (e.g., of SEQ ID NO: 1);
(2) substantially preserves (e.g., having at least about 50%, 60%, 70%, 72.5%, 75%, 77.5%, 80%, 82.5%, 85%, 87.5%, 90%, 92.5%, 95%, 96%, 97%, 98%, 99%, or more of) the spacer sequence-specific cleavage activity of the reference Cas13f polypeptide (e.g., of SEQ ID NO: 1) towards a target RNA complementary to the spacer sequence; and
(3) substantially lacks (e.g., having no more than about 50%, 45%, 40%, 35%, 30%, 27.5%, 25%, 22.5%, 20%, 17.50%, 15%, 12.50%, 10%, 7.50%, 5%, 4.5%, 4%, 3.50%, 3%, 2.50%, 2%, 1.5%, 1% or less of) the spacer sequence-independent collateral cleavage activity of the reference Cas13f polypeptide (e.g., of SEQ ID NO: 1) towards a non-target RNA that does not bind to the spacer sequence.

In some embodiments, the region includes residues within 140, 130, 120, 110, 100, 90, 80, 70, 60, 50, 40, 30, 20, or 10 amino acids from any residues of the N-terminal endonuclease catalytic RXXXXH motif or the C-terminal endonuclease catalytic RXXXXH motif.

In some embodiments, the region includes residues more than 100, 110, 120, or 130 residues away from any residues of the N-terminal endonuclease catalytic RXXXXH motif or the C-terminal endonuclease catalytic RXXXXH motif but are spatially within about 1 to about 10 or about 5 Angstrom of any residue of the N-terminal endonuclease catalytic RXXXXH motif or the C-terminal endonuclease catalytic RXXXXH motif.

In some embodiments, the region comprises, consists essentially of, or consists of residues corresponding to the HEPN1 domain (e.g., residues 1-168), the IDL domain (e.g., residues 168-185), the Helical1 domain (e.g., Helical1-1 (Hell-1) domain (e.g., residues 185-234), Helical1-2 (Hell-2) domain (e.g., residues 281-346), Helical1-3 (Hell-3) domain (e.g., residues 477-644)), the Helical2 domain (e.g., residues 346-477), or the HEPN2 domain (e.g., residues 644-790) of the reference Cas13f polypeptide of SEQ ID NO: 1.

In some embodiments, the mutation comprises, consists essentially of, or consists of, within a stretch of about 8 to about 20 (e.g., about 9 or about 17) consecutive amino acids within the region,
(a) substitution(s) of one or more (e.g., 1, 2, 3, 4, 5, or more) non-Ala (A) residues to Ala (A) residues;
(b) substitution(s) of one or more (e.g., 1, 2, 3, 4, 5, or more) charged residues, nitrogen-containing side chain group residues, bulky (such as F or Y) residues, aliphatic residues, and/or polar residues to charge-neutral short chain aliphatic residues (such as A, V, or I);
(c) substitution(s) of one or more (e.g., 1, 2, 3, 4, 5, or more) Ile (I) and/or Leu (L) residues to Ala (A) residues; and/or
(d) substitution(s) of one or more (e.g., 1, 2, 3, 4, 5, or more) Ala (A) residues to Val (V) residues.

In some embodiments, the one or more non-Ala residues and/or the one or more charged or polar residues comprise N, Q, R, K, H, D, E, Y, S, T, L residues or a combination thereof.

In some embodiments, the one or more non-Ala residues and/or the one or more charged or polar residues comprise N, Q, R, K, H, D, Y, L residues or a combination thereof.

In some embodiments, one or more Y residue(s) within the stretch is substituted.

In some embodiments, the one or more Y residues(s) correspond to Y666 and/or Y677 of the reference Cas13f polypeptide of SEQ ID NO: 1.

In some embodiments, one or more D residue(s) within the stretch is substituted.

In some embodiments, the one or more D residues(s) correspond to D160 and/or D642 of the reference Cas13f polypeptide of SEQ ID NO: 1.

In some embodiments, the charge-neutral short chain aliphatic residue is Ala (A).

In some embodiments, the mutation comprises, consists essentially of, or consists of:
(a) substitutions within 1, 2, 3, 4, or 5 of the stretches of about 8 to about 20 (e.g., about 9 or about 17) consecutive amino acids within the region;
(b) a mutation corresponding to a mutation (e.g., any one in Tables 1-5) that results in an engineered Cas13f polypeptide having at least about 75% of a spacer sequence-specific cleavage activity and no more than about 25% of a spacer sequence-independent collateral cleavage activity, or a combination thereof; and/or
(c) a mutation corresponding to the F7V2, F10V1, F10V4, F40V4, F40S22, F40S26, F40S36, F10S21, F10S24, F10S26, F10S27, F10S33, F10S34, F10S35, F10S36, F10S45, F10S46, F10S48, F10S49, F40S23, or F40S27 mutation in Table 5, or a combination thereof.

In some embodiments, the engineered Cas13f polypeptide retains at least about 50%, 60%, 70%, 72.5%, 75%, 77.5%, 80%, 82.5%, 85%, 87.5%, 90%, 92.5%, 95%, 96%, 97%, 98%, 99%, or more of the spacer sequence-specific cleavage activity of the reference Cas13f polypeptide of SEQ ID NO: 1 towards the target RNA.

In some embodiments, the engineered Cas13f polypeptide has no more than 50%, 45%, 40%, 35%, 30%, 27.50%, 250%, 22.50%, 20%, 17.50%, 150%, 12.50%, 10%, 7.5%, 5%, 4.5%, 4%, 3.5%, 3%, 2.50%, 2%, 1.5%, 1%, or less of the spacer sequence-independent collateral cleavage activity of the reference Cas13f polypeptide of SEQ ID NO: 1 towards the non-target RNA.

In some embodiments, the engineered Cas13f polypeptide has at least about 80% of the spacer sequence-specific cleavage activity of the reference Cas13f polypeptide of SEQ ID NO: 1 towards the target RNA and no more than about 40% of the spacer sequence-independent collateral cleavage activity of the reference Cas13f polypeptide of SEQ ID NO: 1 towards the non-target RNA.

In some embodiments, the mutation is F40S23 (i.e., Y666A/Y677A double mutation).

In some embodiments, the engineered Cas13f polypeptide comprises, consists essentially of, or consists of the amino acid sequence of SEQ ID NO: 3.

In some embodiments, the engineered Cas13f polypeptide further comprises a mutation corresponding to a combination of any one, two, or more (e.g., 3, 4, or 5 more) mutations in Table 6 (such as, D160A, D642A, and/or L641A).

In some embodiments, the mutation is a combination of any one, two, or more (e.g., 3, 4, or 5 more) single mutations in Table 6 (such as, D160A, D642A, and/or L641A) with F40S23 (i.e., Y666A/Y677A double mutation).

In some embodiments, the mutation is a Y666A/Y677A double mutation in combination with 1, 2, or 3 mutations selected from D160A, L641A, and D642A.

In some embodiments, the mutation is any combination mutations in Tables 7-12.

In some embodiments, the mutation is a D160A/D642A/Y666A/Y677A quadruple mutation.

In some embodiments, the engineered Cas13f polypeptide has increased spacer sequence-specific cleavage activity than that of the engineered Cas13f polypeptide of SEQ ID NO: 3.

In some embodiments, the mutation is a mutation corresponding to a combination of a mutation in Tables 13-16 with D160A/D642A/Y666A/Y677A mutation.

In some embodiments, the engineered Cas13f polypeptide comprises, consists essentially of, or consists of the amino acid sequence of SEQ ID NO: 4.

In some embodiments, the engineered Cas13f polypeptide further comprises an amino acid substitution of a non-basic amino acid residue to Arg (R) residue.

In some embodiments, the engineered Cas13f polypeptide further comprises a mutation corresponding to a combination of any one, two, or more (e.g., 3, 4, or 5 more) single mutations in Tables 13-16.

In some embodiments, the engineered Cas13f polypeptide has increased spacer sequence-specific cleavage activity than that of the engineered Cas13f polypeptide of SEQ ID NO: 4.

In some embodiments, the engineered Cas13f polypeptide has a sequence identity of at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% and less than 100% to the reference Cas13f polypeptide of SEQ ID NO: 1.

In some embodiments, the engineered Cas13f polypeptide further comprises a nuclear localization signal (NLS) sequence or a nuclear export signal (NES).

In some embodiments, the engineered Cas13f polypeptide further comprises an N- and/or a C-terminal NLS.

Another aspect of the disclosure provides a polynucleotide encoding the engineered Cas13f polypeptide of the disclosure.

In some embodiments, the polynucleotide is codon-optimized for expression in a eukaryote, a mammal, such as a human or a non-human mammal, a plant, an insect, a bird, a reptile, a rodent (e.g., mouse, rat), a fish, a worm/nematode, or a yeast.

Another aspect of the disclosure provides a CRISPR-Cas13f system comprising:
a) the engineered Cas13f polypeptide of the disclosure or a polynucleotide coding sequence (e.g., a DNA coding sequence or an RNA coding sequence) thereof; and
b) a guide RNA (gRNA) or a polynucleotide coding sequence (e.g., a DNA coding sequence or an RNA coding sequence) thereof, the gRNA comprising:
i. a direct repeat (DR) sequence capable of forming a complex with the engineered Cas13f polypeptide; and,
ii. a spacer sequence capable of hybridizing to a target RNA, and guiding or recruiting the complex to the target RNA.

In some embodiments, the DR sequence has substantially the same secondary structure of that of SEQ ID NO: 2.

In some embodiments, the spacer sequence is in a length of at least 15 nucleotides. In some embodiments, the spacer sequence is in a length of 30 nucleotides.

Another aspect of the disclosure provides a vector comprising the polynucleotide of the disclosure.

In some embodiments, the polynucleotide is operably linked to a promoter. In some embodiments, the polynucleotide is operably linked to an enhancer.

In some embodiments, the promoter is a constitutive promoter, an inducible promoter, a ubiquitous promoter, or a cell, tissue, or organ specific promoter.

In some embodiments, the vector is a plasmid.

In some embodiments, the vector is a retroviral vector, a phage vector, an adenoviral vector, a herpes simplex viral (HSV) vector, an AAV vector, or a lentiviral vector.

In some embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, AAV 13, AAV.PHP.eB, or AAV-DJ.

In some embodiments, the AAV vector is an RNA-encapsulated AAV vector.

Another aspect of the disclosure provides a delivery system comprising (1) a delivery vehicle, and (2) the engineered Cas13f polypeptide of the disclosure, the polynucleotide of the disclosure, CRISPR-Cas13f system of the disclosure, or the vector of the disclosure.

In some embodiments, the delivery vehicle is a nanoparticle (e.g., LNP), a liposome, an exosome, a microvesicle, or a gene-gun.

Another aspect of the disclosure provides a cell or a progeny thereof, comprising the engineered Cas13f polypeptide of the disclosure, the polynucleotide of the disclosure, CRISPR-Cas13f system of the disclosure, the vector of the disclosure, or the delivery system of the disclosure.

In some embodiments, the cell is a eukaryotic cell (e.g., a non-human mammalian cell, a human cell, or a plant cell) or a prokaryotic cell (e.g., a bacteria cell).

Another aspect of the disclosure provides a non-human multicellular eukaryote comprising the cell or progeny of the disclosure.

In some embodiments, the non-human multicellular eukaryote is an animal (e.g., rodent or primate) model for a human genetic disorder.

Another aspect of the disclosure provides a method of modifying a target RNA, the method comprising contacting the target RNA with the CRISPR-Cas13f system of the disclosure, the vector of the disclosure, the delivery system of the disclosure, or the cell or progeny of the disclosure.

In some embodiments, the target RNA is modified by cleavage by the engineered Cas13f polypeptide.

In some embodiments, the target RNA is an mRNA, a tRNA, an rRNA, a non-coding RNA, a lncRNA, or a nuclear RNA.

In some embodiments, upon binding of the complex of the engineered Cas13f polypeptide and the guide RNA to the target RNA, the engineered Cas13f polypeptide does not exhibit substantial (or detectable) spacer sequence-independent collateral cleavage activity.

In some embodiments, the target RNA is within a cell.

In some embodiments, the cell is a cancer cell.

In some embodiments, the cell is infected with an infectious agent.

In some embodiments, the infectious agent is a virus, a prion, a protozoan, a fungus, or a parasite.

In some embodiments, the cell is a neuronal cell (e.g., astrocyte, glial cell (e.g., Muller glia cell, oligodendrocyte, ependymal cell, Schwan cell, NG2 cell, or satellite cell)).

In some embodiments, the CRISPR-Cas13f system is encoded by a first polynucleotide encoding the engineered Cas13f polypeptide, and a second polynucleotide comprising or encoding the guide RNA, wherein the first and the second polynucleotides are introduced into the cell.

In some embodiments, the first and the second polynucleotides are introduced into the cell by the same vector.

In some embodiments, the contacting causes one or more of: (i) in vitro or in vivo induction of cellular senescence; (ii) in vitro or in vivo cell cycle arrest; (iii) in vitro or in vivo cell growth inhibition; (iv) in vitro or in vivo induction of anergy; (v) in vitro or in vivo induction of apoptosis; and (vi) in vitro or in vivo induction of necrosis.

Another aspect of the disclosure provides a method of treating a condition or disease in a subject in need thereof, the method comprising administering to the subject a composition comprising the CRISPR-Cas13f system of the disclosure, the vector of the disclosure, the delivery system of the disclosure, or the cell or progeny of the disclosure; wherein upon administrating, the engineered Cas13f polypeptide cleaves the target RNA, thereby treating the condition or disease in the subject.

In some embodiments, the condition or disease is a neurological condition, a cancer, an infectious disease, or a genetic disorder.

In some embodiments, the cancer is Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

In some embodiments, the neurological condition is glaucoma, age-related RGC loss, optic nerve injury, retinal ischemia, Leber's hereditary optic neuropathy, a neurological condition associated with degeneration of RGC neurons, a neurological condition associated with degeneration of functional neurons in the striatum of a subject in need thereof, Parkinson's disease, Alzheimer's disease, Huntington's disease, Schizophrenia, depression, drug addiction, movement disorder such as chorea, choreoathetosis, and dyskinesias, bipolar disorder, Autism spectrum disorder (ASD), or dysfunction.

In some embodiments, the method is an in vitro method, an in vivo method, or an ex vivo method.

Another aspect of the disclosure provides a CRISPR-Cas13f complex comprising the engineered Cas13f polypeptide of the disclosure, and a guide RNA comprising a DR sequence that binds the engineered Cas13f polypeptide and a spacer sequence capable of hybridizing to a target RNA, and guiding or recruiting the complex to the target RNA.

In some embodiments, the target RNA is encoded by a eukaryotic DNA.

In some embodiments, the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, or a yeast DNA.

In some embodiments, the target RNA is an mRNA.

In some embodiments, the CRISPR-Cas13f complex further comprises a target RNA comprising a sequence capable of hybridizing to the spacer sequence.

It should be understood that any one embodiment of the disclosure described herein, including those described only in the examples or claims, or only in one aspects/sections below, can be combined with any other one or more embodiments of the disclosure, unless explicitly disclaimed or improper.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 shows a view of the predicted 3D structure (by I-TASSER) of the reference Cas13f polypeptide of SEQ ID NO: 1 in ribbon representation. The RXXXXH motifs of the two HEPN domains are the catalytic sites.

FIG. 2 is the schematic drawing of an exemplary one-plasmid mammalian dual-fluorescence reporter system for detecting cleavage and collateral activities of Cas13f mutants.

FIG. 3 shows 20 segments in HEPN1, HEPN2, IDL, and Hell-3 domains of reference Cas13f polypeptide of SEQ ID NO: 1 selected for mutagenesis, with each spanning 9 or 17 amino acids.

FIG. 4 shows the percentages of EGFP or mCherry⁺ cells for Cas13f mutants normalized to dead Cas13f (dCas13f).

FIG. 5 shows the percentages of EGFP⁺ or mCherry⁺ cells for Cas13f mutants with combination mutations in or nearby F10V1, F10V4, F38V2, F40V2, F40V4, F46V1 and F46V3 normalized to dead Cas13f(dCas13f).

FIG. 6 is the schematic drawing of an exemplary two-plasmid mammalian dual-fluorescence reporter system for detecting cleavage and collateral activities of Cas13f mutants.

FIG. 7 shows quantification of MFIs (mean fluorescence intensities) of EGFP and mCherry for Cas13f mutants normalized to NT.

FIG. 8 shows the SOD1 mRNA knockdown efficiency of Cas13f mutants in Cos7 cells normalized to NT.

FIG. 9 shows quantification of MFIs of EGFP and mCherry for Cas13f mutants normalized to NT.

FIG. 10 shows quantification of MFIs of EGFP and mCherry for Cas13f mutants normalized to NT.

FIG. 11 shows the SOD1 mRNA knockdown efficiency of Cas13f v2, v3, v2+H638A&D642A mutants in Cos7 cells normalized to NT.

FIG. 12 shows the functional domain structure of Cas13f v3. The four amino acid mutations marked in red are the mutations of Cas13f v3 compared with the reference Cas13f polypeptide.

FIG. 13 is the schematic drawing of an exemplary mammalian fluorescence reporter system for detecting the cleavage activities of Cas13f mutants.

FIG. 14 Mean fluorescence intensity of RFP of BFP positive cells for Cas13f mutants normalized to the non-targeting negative control (“NT”). All values are presented as mean±s.d. (n=2), *P<0.05, **P<0.01.

FIG. 15 Mean fluorescence intensity of RFP of BFP positive cells for Cas13f mutants normalized to the non-targeting negative control (“NT”). All values are presented as mean±s.d. (n=2 or 1), *P<0.05, **P<0.01.

FIG. 16 Mean fluorescence intensity of RFP of BFP positive cells for Cas13f mutants normalized to the non-targeting negative control (“NT”). All values are presented as mean±s.d. (n=2), *P<0.05, **P<0.01.

FIG. 17 Mean fluorescence intensity of RFP of BFP positive cells for Cas13f mutants normalized to the non-targeting negative control (“NT”). All values are presented as mean±s.d. (n=2 or 1), *P<0.05, **P<0.01.

The figures herein are for illustrative purposes only and are not necessarily drawn to scale.

### DETAILED DESCRIPTION OF THE EXAMPLE EMBODIMENTS

### 1. Overview

Several subtypes of Class 2 type VI exist, including at least subtype VI-A (Cas13a/C2c2), VI-B (Cas13b1 and Cas13b2), VI-C(Cas13c), VI-D (Cas13d, CasRx), VI-E (Cas13e), and VI-F (Cas13f). The Cas13 subtypes generally share very low sequence identity/similarity, but can all be classified as type VI Cas proteins (e.g., generally referred to herein as “Cas13”) based on the presence of two conserved HEPN-like RNase domains. Although these two domains appear to be a conserved feature of Cas13 enzymes and are typically located close to the two terminal ends, their spacing within the protein appears to be unique for each subtype. At least three crystal structures for type VI-A Cas13a proteins have been published, including Cas13a from Leptotrichia shahii (LshCas13a), Lachnospiraceae bacterium (LbaCas13a), and Leptotrichia buccalis (LbuCas13a). Similar to other Class 2 complexes, the crRNA-Cas13a complex is bi-lobed with a nuclease (NUC) lobe and a crRNA recognition (REC) lobe. The crRNA-bound form of Cas13a adopts a “clenched fist”-like structure, with the REC lobe being imperfectly stacked on top of the NUC lobe. The REC lobe has a variable N-terminal domain (NTD), followed by a helical domain (Helical-1). Meanwhile, the NUC lobe consists of the two HEPN domains (HEPN-1 and HEPN-2) separated by a linker domain (Helical-3). In addition, the HEPN-1 domain is split into two subdomains by another helical domain (Helical-2). The NTD, Helical-1, and HEPN2 domains form a narrow, positively charged cleft that anchors the 5′ repeat-derived end of the bound crRNA (the 5′-handle), whereas the 3′ end of the crRNA is bound by the Helical-2 domain.

The Cas13 CRISPR locus is initially transcribed into a long pre-crRNA transcript. The Cas13 proteins then cleave the pre-crRNA at fixed positions upstream of the stem-loop structure formed by the palindromic nature of the direct repeat (DR) sequences. Pre-crRNA processing in type VI involves metal-independent cleavages upstream of the stem-loop, and does not require a trans-activating crRNA (tracrRNA) or other host factors. The mature crRNA, which comprises a DR sequence and a guide sequence complementary to a target RNA, assembles with the Cas13 proteins to form a functional RNP complex, which then scans transcripts for the complementary RNA target. Once such RNA target is found and bound by the guide sequence, the RNA target is degraded by the Cas13 endonuclease.

The Cas13 effector proteins display unprecedented sensitivity to recognize specific target RNAs within a heterogeneous population of non-target RNAs. It has been reported that Cas13 can detect target RNAs with femtomolar sensitivity. Thus, on the one hand, the Class 2 type VI enzymes or Cas13 offer tremendous opportunity to knock down target gene products (e.g., mRNA) for gene therapy, yet on the other hand, such use is inherently limited by the co-called collateral activity that poses significant risk of cytotoxicity.

Specifically, in Class 2 type VI systems, a guide sequence non-specific RNA cleavage, referred to as “collateral activity,” is conferred by the higher eukaryotes and prokaryotes nucleotide-binding (HEPN) domain in Cas13 after target RNA binding. Binding of its cognate target ssRNA complementary to the bound crRNA causes substantial conformational changes in Cas13f effector protein, leading to the formation of a single, composite catalytic site for guide sequence independent “collateral” RNA cleavage, thus converting Cas13 into a sequence non-specific ribonuclease. This newly formed highly accessible active site would not only degrade the target RNA in cis if the target RNA is sufficiently long to reach this new active site, but also degrade non-target RNAs in trans based on this promiscuous RNase activity.

Most RNAs appear to be vulnerable to this promiscuous RNAse activity of Cas13f, and most (if not all) Cas13f effector proteins possess this collateral cleavage activity. It has been shown recently that the collateral effects by Cas13-mediated knockdown exist in mammalian cells and animals (manuscript submitted), suggesting that clinical application of Cas13-mediated target RNA knock down will face significant challenge in the presence of collateral effect.

The existence of substantial collateral effects of Cas13-mediated RNA knockdown has been demonstrated using a dual-fluorescent reporter system of the disclosure as described herein. Such collateral effects have been observed for both exogenous and endogenous genes in mammalian cells.

Thus, in order to use the Cas13 enzymes for specifically knocking down a target RNA in gene therapy, it is evident that this guide sequence non-specific collateral activity must be tightly controlled to prevent unwanted spontaneous cellular toxicity. Through unclear mechanism, subtype VI-B systems include a natural means to regulate the collateral activity of Cas13b via the type VI-associated genes csx27 and csx28, but such natural regulatory mechanism appears to be unique to subtype VI-B, as similar mechanism does not seem to exist in other subtypes such as type VI-A and VI-C.

Using the reporter system of the disclosure, it was found that several mutants with 2-4 mutations on the Higher Eukaryotes and Prokaryotes Nucleotide-binding (HEPN) domains retained undiminished on-target activity, but greatly reduced collateral effects.

Interestingly, it was found that the majority of mutants exhibited either low dual cleavage activity, or high on-target cleavage activity but low collateral cleavage activity. However, there is almost no mutants showing low on-target cleavage activity but high collateral cleavage activity. These results suggest a distinct binding mechanism between on-target and collateral cleavage activity.

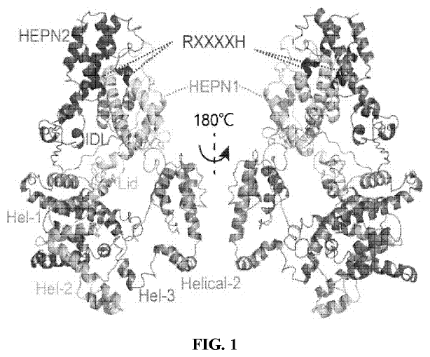

While not wishing to be bound by any particular theory, Applicant believes the following model of target (e.g., gRNA-specific) and collateral cleavage activity aids the rationale design of collateral effect-free mutants of the Cas13f effector proteins. Specifically, as shown in FIG. 1, Cas13f is believed to contain two separated binding domains proximal to the HEPN domains—one is responsible for on-target cleavage, and both are required for collateral cleavage. Consistent with this model, mutations designed on the F10, F38, and F40 regions, surrounding the cleavage site, cause steric hindrance effects or change in charge, leading to weakened interactions between activated Cas13f and promiscuous RNA, but not much (if any) effect between activated Cas13f and the on-target RNA. Thus, mutagenesis on these binding sites abolishes the collateral cleavage activity of Cas13f, while retaining the on-target cleavage activity of the corresponding wild type Cas13f.

Thus, the disclosure described herein provides engineered high-fidelity Class 2 type VI or Cas13f effector protein mutants with minimal residual collateral effects. These mutants are useful, for example, in targeting degradation of RNAs in basic research and therapeutic applications.

On the other hand, multiple low-fidelity Cas13f mutants exhibiting increased dual cleavage activity were identified. Such mutants have utility for better nucleic acid detection application (such as those used in the SHERLOCK assay).

Specifically, in one aspect, the disclosure provides engineered Class 2 type VI or Cas13f effector proteins that largely maintain their sequence-specific cleavage activity against a target RNA, yet with diminished if not eliminated non-guide sequence-specific cleavage activity against non-target RNAs. Such engineered Cas13f effector proteins that substantially lack collateral effect pave the way for using Cas13f in target RNA-knock down-based utility, such as gene therapy. Such engineered Cas13f effector proteins that substantially lack collateral effect are also useful for RNA-base editing, because a nuclease dead version (or “dCas13”) of such engineered Cas13f also has reduced off-target effect, which is still present in dCas13f without the mutations in the subject engineered Cas13f.

Wild type Cas13f not only possesses the ability to bind a target RNA through the guide sequence of the crRNA, but also possesses a non-specific RNA binding site (see the oval shaped motif around the catalytic site) for any RNA at the vicinity of the HEPN catalytic domains. Once the target RNA is recognized by the guide sequence, a conformation change of Cas13f activates its catalytic activity, and the target RNA, bound by both the complementary guide sequence and the non-specific RNA binding site, is cleaved. Once activated, Cas13f also non-specifically cleave non-target RNA that does not bind to the guide sequence, partly due to the binding of such non-target RNA to the non-specific RNA binding site on cas13. Mutations in the non-specific RNA binding motif (as signified by a different shade of the oval motif) reduces/eliminates (or in some cases enhances) the ability of Cas13f to bind RNA, thus collateral activity against non-target RNA is reduced/eliminated (or enhanced) without significantly affecting target RNA cleavage because the target RNA is still bound by the guide sequence.

According to this model, off-target effect in RNA-base editing using a nuclease-deficient (dCas13) version of the engineered Cas13f can also be reduced or eliminated, because the loss of non-specific RNA binding in the engineered dCas13f reduced/eliminates unintended RNA based editing due to the proximity of the RNA base editing domain (e.g., ADAR or CDAR) and an off-target RNA substrate.

In a related aspect, the disclosure also provides engineered Class 2 type VI or Cas13f effector proteins that largely maintain their sequence-specific cleavage activity against a target RNA, yet with enhanced non-guide sequence-specific cleavage activity against non-target RNAs compared to the corresponding wild type Cas13f. Such engineered Cas13f with enhanced collateral effect provides a better (e.g., more sensitive) mutant, compared to the wild type, in nucleic acid detection assays such as SHERLOCK, which takes advantage of the collateral activity to provide an extreme sensitive assay for detecting very small quantities of a guide sequence-specific target RNA in a sample, with or without pre-amplification of the initial nucleic acids in the sample.

More specifically, one aspect of the disclosure provides an engineered Class 2 type VI Clustered Regularly Interspaced Short Palindromic Repeat (CRISPR)-Cas13f effector, wherein the engineered Class 2 type VI Cas effector protein: (1) comprises a mutation in a region spatially close to an endonuclease catalytic domain of the corresponding wild type effector protein; (2) substantially preserves guide sequence-specific endonuclease cleavage activity of the wild type effector protein (or theoretical maximum thereof) towards a target RNA complementary to the guide sequence; and, (3) either substantially lacks or has enhanced guide sequence-independent collateral endonuclease cleavage activity of the wild type effector protein (or theoretical maximum thereof) towards a non-target RNA that is substantially not complement to/does not bind to the guide sequence.

In certain embodiments, the guide sequence-specific endonuclease cleavage activity and the guide sequence-independent collateral endonuclease cleavage activity can both be measured as compared to the corresponding wild type Cas13f effector proteins, as normalized against a corresponding nuclease-deficient (catalytically inactive) Cas13f (such as dCas13f).

The nuclease-deficient Cas13f may be lack of catalytic domain, motif, or key catalytic residues such that it exhibits no appreciable or detectable level of guide sequence-dependent target RNA endonuclease cleavage activity, as well as guide sequence-independent collateral endonuclease cleavage activity. Thus in the due reporter system described herein, dCas13f typically has 100% remaining/baseline EGFP signal as an indication of no appreciable or detectable level of guide sequence-dependent target RNA endonuclease cleavage activity, and has 100% remaining/baseline mCherry signal as an indication of no appreciable or detectable level of guide sequence-independent collateral endonuclease cleavage activity. Meanwhile, wild type Cas13f typically exhibit strong guide sequence-dependent target RNA endonuclease cleavage activity (as reflected by nearly 80%, 90%, 95%, or close to 100% reduction of the dCas13f EGFP reference signal). The theoretical maximum of such guide sequence-dependent target RNA endonuclease cleavage activity is 100%, which is equivalent to complete elimination of all dCas13f EGFP reference signal.

Wild-type Cas13f also typically exhibit various levels of guide sequence-independent collateral endonuclease cleavage activity, leading to about 50%-70% reduction of the dCas13f mCherry reference signal. The theoretical maximum of such guide sequence-independent collateral endonuclease cleavage activity is 100%, which is equivalent to complete elimination of all dCas13f mCherry reference signal. In certain embodiments, the engineered Cas13f effector protein of the disclosure exhibits reduced or diminished guide sequence-independent collateral endonuclease cleavage activity compared to the corresponding wild type Cas13f (or theoretical maximum thereof) from which the engineered Cas13f derives. For example, the engineered Cas13f effector protein may substantially lack (e.g., retains less than 50%, 40%, 35%, 30%, 27.50%, 250%, 22.50%, 20%, 17.50%, 150%, 12.50%, 10%, 7.5%, 5%, 4%, 3%, 2.5%, 2%, 1% or less of) guide sequence-independent collateral endonuclease cleavage activity of the wild type Cas13f towards a non-target RNA that does not bind to the guide sequence. For example, if the wild type Cas13f eliminates about 70% (with the theoretical maximum being 100% elimination) of the dCas13f mCherry baseline signal due to collateral activity, and the mutant Cas13f with diminished collateral activity only eliminates about 10% of the dCas13f mCherry baseline signal due to remaining collateral activity, the mutant only exhibits or retains about 1/7 (or about 150%) of the wild type collateral activity (or 10% of the theoretical maximum).

In certain embodiments, the engineered Cas13f effector protein of the disclosure exhibits increased or enhanced guide sequence-independent collateral endonuclease cleavage activity compared to the corresponding wild type Cas13f from which the engineered Cas13f derives. For example, the engineered Cas13f effector protein may have substantially enhanced or increased (e.g., has more than 100%, 110%, 120%, 130%, 140%, 150%, 160%, 170%, 180%, 190%, 200% or more of) guide sequence-independent collateral endonuclease cleavage activity of the wild type Cas13f towards anon-target RNA that does not bind to the guide sequence. For example, if the wild type Cas13f eliminates about 50% of the dCas13f mCherry baseline signal due to collateral activity, and the mutant Cas13f with enhanced collateral activity eliminates about 90% of the dCas13f mCherry baseline signal due to its enhanced collateral activity, the mutant exhibits about 90/50 (or about 180%) of the wild type collateral activity.

In certain embodiments, the mutation occurs within a region, e.g., within one of two RNA binding domains at, near, or proximal to one of the HEPN-type catalytic domains, of a wild type Cas13f. In certain embodiments, the mutation weakens (e.g., significantly weakens or eliminates) binding of the wild type Cas13f to a non-specific RNA target (e.g., one not substantially complementary to a guide RNA), but substantially retains binding to a target RNA substantially complementary to the guide RNA. In certain embodiments, the mutation causes steric hindrance effects and/or change in charge, polarity, and/or size of the sidechain of the involved residues, leading to weakened interactions between activated Cas13f and promiscuous RNA, but not much (if any) effect between activated Cas13f and the on-target RNA.

As used herein, “Cas13” is a Class 2 type VI CRISPR-Cas effector protein that displays collateral activity as wild type enzyme upon binding to a cognate target RNA complementary to a guide sequence of its crRNA. The collateral activity of a wild type Class 2 type VI effector protein enables it to cleave RNase or endonuclease activity against a non-target RNA that does not or substantially does not complement with the guide sequence of the crRNA. The wild type Class 2 type VI effector protein may also exhibit one or more of the following characteristics: having one or two conserved HEPN-like RNase domains, such as HEPN domains having the conserved RXXXXH motif (with X being any amino acid), e.g., the RXXXXH motifs described herein below; having a “clenched fist”-like structure when the Class 2 type VI effector protein (e.g., Cas13) binds a cognate crRNA; having a bi-lobed structure with a nuclease (NUC) lobe and a crRNA recognition (REC) lobe, optionally, the REC lobe has a variable N-terminal domain (NTD), followed by a helical domain (Helical-1), and/or optionally, the NUC lobe consists of the two HEPN domains (HEPN-1 and HEPN-2) separated by a linker domain (Helical-3), wherein the HEPN-1 domain is optionally split into two subdomains by another helical domain (Helical-2); processes pre-crRNA transcript into crRNA; does not require a trans-activating crRNA (tracrRNA) or other host factors for pre-crRNA processing; and exhibits femtomolar sensitivity to recognize guide sequence-specific target RNAs within a heterogeneous population of non-target RNAs.

In certain embodiments, the Class 2 type VI effector protein (e.g., Cas13) has one of the RXXXXN motifs in the HEPN-like domains located at or close to (e.g., within 50-160 residues, or within 10, 20, 30, 40, 50, 60, 70, 80, 90, 100, 110, 120, 130, 140, 150 or 160 residues of) the N-terminus. In certain embodiments, the Class 2 type VI effector protein (e.g., Cas13) has one of the RXXXXN motifs in the HEPN-like domains located at or close to (e.g., within 50-160 residues, or within 10, 20, 30, 40, 50, 60, 70, 80, 90, 100, 110, 120, 130, 140, 150 or 160 residues of) the C-terminus. In certain embodiments, the Class 2 type VI effector protein (e.g., Cas13) has one of the RXXXXN motifs of the HEPN-like domains located at or close to (e.g., within 50-160 residues, or within 10, 20, 30, 40, 50, 60, 70, 80, 90, 100, 110, 120, 130, 140, 150 or 160 residues of) the N-terminus, while the other of the RXXXXN of the HEPN-like domains is located at or close to (e.g., within 50-160 residues, or within 10, 20, 30, 40, 50, 60, 70, 80, 90, 100, 110, 120, 130, 140, 150 or 160 residues of) the C-terminus. An RXXXXN motif is “at or near” the N- or C-terminus, if either the R or the N residue of the RXXXXN motif is at or near the N- or C-terminus.

Based on biological and cellular experimental data, the engineered Cas13f effector proteins have drastically reduced non-sequence-specific cleavage activity against non-target RNAs, yet simultaneously exhibiting substantially the same if not higher sequence-specific cleavage activity against a target RNA that substantially complements the guide sequence of the crRNA. The engineered effector proteins enable high fidelity RNA targeting/editing.

In certain embodiments, the Cas13f effector protein is a Cas13f effector protein, or an ortholog, paralog, homolog, natural or engineered mutant thereof, or functional fragment thereof that substantially maintains the guide sequence-specific cleavage activity.

In certain embodiments, the mutant or functional fragment thereof maintains at least one function of the corresponding wild type effector protein. Such functions include, but are not limited to, the ability to bind a guide RNA/crRNA of the disclosure (described herein below) to form a complex, the guide sequence-specific RNase activity, and the ability to bind to and cleave a target RNA at a specific site under the guidance of the crRNA that is at least partially complementary to the target RNA.

In some embodiments, the Cas13f protein is a wild type or reference Cas13f polypeptide. In certain embodiments, the wild type or reference Cas13f polypeptide has an amino acid sequence of SEQ ID NO: 1 (Cas13f.1) of the disclosure, any one of SEQ ID NOs: 2-7 (Cas13f.2, Cas13f.3, Cas13f.4, and Cas13f.5, respectively) of PCT/CN2020/077211, incorporated herein by reference in its entirety, or any one of SEQ ID NOs: 9-10 (Cas13f.6 and Cas13f.7, respectively) of PCT/CN2022/101884, incorporated herein by reference in its entirety. The direct repeat (DR) sequences for those wild type or reference Cas13f polypeptides are SEQ ID NO: 2 (Cas13f.1) of the disclosure, any one of SEQ ID NOs: 11-14 (Cas13f.2, Cas13f.3, Cas13f.4, and Cas13f.5, respectively) of PCT/CN2020/077211, incorporated herein by reference in its entirety, or any one of SEQ ID NOs: 26-27 (Cas13f6 and Cas13f7, respectively) of PCT/CN2022/101884, incorporated herein by reference in its entirety, respectively.

As used herein, “direct repeat sequence” may refer to the DNA coding sequence in the CRISPR locus, or to the RNA encoded by the same in crRNA. Thus when such a sequence is referred to in the context of an RNA molecule, such as crRNA, each T is understood to represent a U.

In certain embodiments, the wild type Cas13f effector proteins of the disclosure can be: (i) SEQ ID NO: 1 (Cas13f.1) of the disclosure, any one of SEQ ID NOs: 2-7 (Cas13f.2, Cas13f.3, Cas13f.4, and Cas13f.5, respectively) of PCT/CN2020/077211, or any one of SEQ ID NOs: 9-10 (Cas13f.6 and Cas13f7, respectively) of PCT/CN2022/101884, such as SEQ ID NO: 1 of the disclosure; (ii) an ortholog, paralog, homolog of SEQ ID NO: 1 (Cas13f.1) of the disclosure, any one of SEQ ID NOs: 2-7 (Cas13f.2, Cas13f.3, Cas13f.4, and Cas13f.5, respectively) of PCT/CN2020/077211, or any one of SEQ ID NOs: 9-10 (Cas13f.6 and Cas13f.7, respectively) of PCT/CN2022/101884; or (iii) a Cas13f effector protein having amino acid sequence identity of at least about 80%, 85%, 90%, 91%, 92%, 93%, 94%, 95%, 96%, 97%, 98%, or 99% compared to any one of SEQ ID NO: 1 (Cas13f.1) of the disclosure, any one of SEQ ID NOs: 2-7 (Cas13f.2, Cas13f.3, Cas13f.4, and Cas13f.5, respectively) of PCT/CN2020/077211, or any one of SEQ ID NOs: 9-10 (Cas13f6 and Cas13f7, respectively) of PCT/CN2022/101884.

In certain embodiments, the Cas13f effector proteins, orthologs, homologs, derivatives, and functional fragments thereof are naturally existing. In certain other embodiments, the Cas13f effector proteins, orthologs, homologs, derivatives, and functional fragments thereof are not naturally existing, e.g., having at least one amino acid difference compared to a naturally existing sequence.

In certain embodiments, the region spatially close to the endonuclease catalytic domain of the corresponding wild type Cas13f effector protein includes residues within 120, 110, 100, 90, 80, 70, 60, 50, 40, 30, 20, or 10 amino acids from any residues of the endonuclease catalytic domain (e.g., an RXXXXH domain) in the primary sequence of the Cas13f.

In certain embodiments, the region includes residues within 140, 130, 120, 110, 100, 90, 80, 70, 60, 50, 40, 30, 20, or 10 amino acids from any residues of the endonuclease catalytic domain (e.g., an RXXXXH domain) in the primary sequence of the Cas13f.

In certain embodiments, the region spatially close to the endonuclease catalytic domain of the corresponding wild type Cas13f effector protein includes residues more than 100, 110, 120, or 130 residues away from any residues of the endonuclease catalytic domain in the primary sequence of the Cas13f but are spatially within 1-10 or 5 angstrom of a residue of the endonuclease catalytic domain.

In certain embodiments, the endonuclease catalytic domain is a HEPN domain, optionally a HEPN domain comprising an RXXXXH motif.

In certain embodiments, the N-terminal RXXXXH motif has a RNFYSH sequence.

In certain embodiments, the C-terminal RXXXXH motif has a RNKALH sequence.

In certain embodiments, region comprises, consists essentially of, or consists of residues corresponding to residues corresponding to the HEPN1 domain (e.g., residues 1-168), Helical1 domain, Helical2 domain (e.g., residues 346-477), and the HEPN2 domain (e.g., residues 644-790) of SEQ ID NO: 1.

In certain embodiments, the mutation comprises, consists essentially of, or consists of substitutions, within a stretch of 8-20 consecutive amino acids within the region, one or more charged or polar residues to a charge neutral short chain aliphatic residue (such as A). For example, in some embodiments, the stretch is about 9 or 17 residues.

In certain embodiments, the mutation comprises, consists essentially of, or consists of substitutions, within a stretch of 15-20 consecutive amino acids within the region, (a) one or more charged, nitrogen-containing side chain group, bulky (such as F or Y), aliphatic, and/or polar residues to a charge-neutral short chain aliphatic residue (such as A, V, or I); (b) one or more I/L to A substitution(s); and/or (c) one or more A to V substitution(s).

In certain embodiments, substantially all, except for up to 1, 2, or 3, charged and polar residues within the stretch are substituted.

In certain embodiments, a total of about 7, 8, 9, or 10 charged and polar residues within the stretch are substituted.

In certain embodiments, the N- and C-terminal 2 residues of the stretch are substituted to amino acids the coding sequences of which contain a restriction enzyme recognition sequence. For example, in some embodiments, the N-terminal two residues may be VF, and the C-terminal 2 residues may be ED, and the restriction enzyme is Bpi1. Other suitable RE sites are readily envisioned. The RE sites for the N- and C-terminal ends can be, but need not be identical.

In certain embodiments, the one or more charged or polar residues comprise N, Q, R, K, H, D, E, Y, S, and T residues. In certain embodiments, the one or more charged or polar residues comprise R, K, H, N, Y, and/or Q residues.

In certain embodiments, the charge-neutral short chain aliphatic residue is A, I, L, V, or G.

In certain embodiments, the charge-neutral short chain aliphatic residue is Ala (A).

In certain embodiments, the mutation comprises, consists essentially of, or consists of substitutions within 2, 3, 4, or 5 the stretches of 15-20 consecutive amino acids within the region.

In certain embodiments, the mutation with reduced collateral activity comprises, consists essentially of, or consists of: (a) substitutions within 1, 2, 3, 4, or 5 of the stretches of 15-20 consecutive amino acids within the region; (b) a mutation corresponds to a Cas13f mutation (e.g., that of Example 1) that retains at least about 75% of guide RNA-specific cleavage of wild type Cas13f (such as SEQ ID NO: 1) (or theoretical maximum thereof), and exhibits less than about 25 or 27.5% collateral effect of wild type Cas13f (such as SEQ ID NO: 1) (or theoretical maximum thereof); (c) a mutation corresponds to the F7V2, F10V1, F10V4, F40V2, F40V4, F44V2, F10S19, F10S21, F10S24, F10S26, F10S27, F10S33, F10S34, F10S35, F10S36, F10S45, F10S46, F10S48, F10S49, F40S22, F40S23, F40S26, F40S27, or F40S36 mutation of Cas13f mutation; (d) a mutation corresponds to a Cas13f mutation (e.g., that of Example 12) that retains between about 50-75% of guide RNA-specific cleavage of wild type Cas13f (such as SEQ ID NO: 1) (or theoretical maximum thereof), (e) exhibits less than about 25%, 27.5%, or 40% collateral effect of wild type Cas13f (such as SEQ ID NO: 1) (or theoretical maximum thereof); and/or (f) a mutation corresponds to the F2V4, F3V1, F3V3, F3V4, F5V2, F5V3, F6V4, F7V1, F38V4, F40V1, F41V1, F41V3, F42V4, F43V1, F10S2, F10S11, F10S12, F10S18, F10S20, F10S23, F10S25, F10S28, F10S43, F10S44, F10S47, F10S50, F10S51, F10S52, F40S7, F40S9, F40S11, F40S21, F40S22, F40S24, F40S28, F40S29, F40S30, F40S35, or F40S37 mutation of Cas13f mutation.

In certain embodiments, the mutation with enhanced collateral activity comprises, consists essentially of, or consists of: (a) substitutions within 1, 2, 3, 4, or 5 of the stretches of 15-20 consecutive amino acids within the region; (b) a mutation corresponds to a Cas13f mutation (e.g., that of Example 1) that retains at least about 75% of guide RNA-specific cleavage of wild type Cas13f (such as SEQ ID NO: 1) (or theoretical maximum thereof), and exhibits more than about 110%, 115%, 120%, 125%, 130%, 135%, 140%, 145%, 150%, 155%, 160%, 165%, 170%, 175%, 180% or more collateral effect of wild type Cas13f (such as SEQ ID NO: 1); and/or (c) a mutation corresponds to the F38V2, F42V1, F46V3, F38S2, F38S4, F38S5, F38S6, F38S7, F38S8, F38S9, F38S10, F38S11, F38S12, F38S13, F38S15, F38S16, F38S17, F40S1, F40S2, F40S3, F40S4, F40S5, F40S6, F40S8, F40S16, F40S18, F46S1, F46S4, F46S6, F46S7, F46S10, F46S14, F46S15, F10S4, F10S5, F10S6, F10S9, F10S10, F10S7, F38S1, F38S13, or F46S2 mutation of Cas13f mutation (e.g., that of Example 1).

The sequences of the mutations and/or mutants referenced herein for Cas13f are described in detail in the examples and the associated sequence listing.

In certain embodiments, more than one (e.g., any combinations of two or more of) such mutations/mutants may be present in the same engineered Cas13f effector protein.

In certain embodiments, the engineered Cas13f preserves at least about 50%, 60%, 70%, 72.5%, 75%, 80%, 85%, 87.5%, 90%, 95%, 96%, 97%, 97.5%, 98%, or 99% of the guide sequence-specific endonuclease cleavage activity of the wild type Cas13f (or theoretical maximum thereof) towards the target RNA.

In certain embodiments, the engineered Cas13f has at least about 95%, 100%, 105%, 110%, 115%, 120%, 125%, 130%, 135%, 140%, 145%, 150%, 155%, 160% or more of the guide sequence-specific endonuclease cleavage activity of the wild type Cas13f towards the target RNA. That is, the subject engineered Cas13f mutant may have higher guide sequence-specific endonuclease cleavage activity towards the target RNA compared to the wild type Cas13f from which the mutant is derived.

In certain embodiments, the engineered Cas13f lacks at least about 70%, 72.5%, 75%, 77.5%, 80%, 82.5%, 85%, 87.5%, 90%, 92.5%, 95%, 96%, 97%, 98%, 99%, or 100% of the guide sequence-independent collateral endonuclease cleavage activity of the wild type Cas13f (or theoretical maximum thereof) towards the non-target RNA.

In certain embodiments, the engineered Cas13f preserves at least about 80-90% of the guide sequence-specific endonuclease cleavage activity of the wild type Cas13f (or theoretical maximum thereof) towards the target RNA, and lacks at least about 95-100% of the guide sequence-independent collateral endonuclease cleavage activity of the wild type Cas13f (or theoretical maximum thereof) towards the non-target RNA.

In certain embodiments, the guide RNA-specific and collateral (gRNA-independent) cleavage activity by the engineered Cas13f effector proteins are measured using methods substantially as described in any of the examples (such as Examples 1, 2, 4, 5 and 12).

In certain embodiments, the amino acid sequence contains up to 1, 2, 3, 4, or 5 differences in one or more segments defined in Table 1 or 2, as compared to the corresponding segment of SEQ ID NO: 1. For example, additional changes in one or more segments defined in Table 1 or 2 are possible without substantially negatively affect the guide sequence-specific cleavage activity, and/or do not increase the guide sequence-independent collateral effect.

In certain embodiments, the engineered Cas13f of the disclosure has the amino acid sequence of SEQ ID NO: 3 or 4.

In certain embodiments, the engineered Cas13f of the disclosure further comprises a nuclear localization signal (NLS) sequence or a nuclear export signal (NES). For example, in certain embodiments, the engineered Cas13f may comprise an N- and/or a C-terminal NLS.

In a related aspect, the disclosure provides additional derivatives of the subject engineered Cas13f, such as those either substantially lacking or having enhanced collateral cleavage activity, such as Cas13f effector proteins based on any one of SEQ ID NOs: 3-4, or the above orthologs, homologs, derivatives, and functional fragments thereof, which comprises another covalently or non-covalently linked protein or polypeptide or other molecules (such as detection reagents or drug/chemical moieties). Such other proteins/polypeptides/other molecules can be linked through, for example, chemical coupling, gene fusion, or other non-covalent linkage (such as biotin-streptavidin binding). Such derived proteins do not affect the function of the original protein, such as the ability to bind a guide RNA/crRNA of the disclosure (described herein below) to form a complex, the RNase activity, and the ability to bind to and cleave a target RNA at a specific site, under the guidance of the crRNA that is at least partially complementary to the target RNA. In addition, such derived proteins do retain the characteristics of the subject engineered Cas13f either lacking or having enhanced collateral cleavage activity.

That is, in certain embodiments, upon binding of the RNP complex of the subject engineered Cas13f (or derivative thereof) to the target RNA, the engineered Cas13f either does not exhibit substantial (or detectable) or has enhanced collateral RNase activity.

Such derivation may be used, for example, to add a nuclear localization signal (NLS, such as SV40 large T antigen NLS (SEQ ID NO: 5)) to enhance the ability of the subject Cas13f effector proteins, to enter cell nucleus. Such derivation can also be used to add a targeting molecule or moiety to direct the subject Cas13f effector proteins, to specific cellular or subcellular locations. Such derivation can also be used to add a detectable label to facilitate the detection, monitoring, or purification of the subject Cas13f effector proteins. Such derivation can further be used to add a deamination enzyme moiety (such as one with adenine or cytosine deamination activity) to facilitate RNA base editing.

The derivation can be through adding any of the additional moieties at the N- or C-terminal of the subject Cas13f effector proteins, or internally (e.g., internal fusion or linkage through side chains of internal amino acids).

In a related aspect, the disclosure provides conjugates of the subject engineered Cas13f, such as those either substantially lacking or having enhanced substantially lacking collateral cleavage activity, such as Cas13f effector proteins based on any one of SEQ ID NOs: 3-4, or the above orthologs, homologs, derivatives, and functional fragments thereof, which are conjugated with moieties such as other proteins or polypeptides, detectable labels, or combinations thereof. Such conjugated moieties may include, without limitation, localization signals, reporter genes (e.g., GST, HRP, CAT, GFP, HcRed, DsRed, CFP, YFP, BFP), labels (e.g., fluorescent dye such as FITC, or DAPI), NLS, targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deaminase domain (e.g., ADARI, ADAR2, APOBEC, AID, or TAD), methylase domain, demethylase domain, transcription release factor, HDAC, a moiety having ssRNA cleavage activity, a moiety having dsRNA cleavage activity, a moiety having ssDNA cleavage activity, a moiety having dsDNA cleavage activity, DNA or RNA ligase domain, or any combination thereof.

For example, the conjugate may include one or more NLSs, which can be located at or near N-terminal, C-terminal, internally, or combination thereof. The linkage can be through amino acids (such as D or E, or S or T), amino acid derivatives (such as Ahx, p-Ala, GABA or Ava), or PEG linkage.

In certain embodiments, conjugations do not affect the function of the original engineered protein, such as those either substantially lacking or having enhanced collateral effect, such as the ability to bind a guide RNA/crRNA of the disclosure (described herein below) to form a complex, and the ability to bind to and cleave a target RNA at a specific site, under the guidance of the crRNA that is at least partially complementary to the target RNA.

In a related aspect, the disclosure provides fusions of the subject engineered Cas13f, such as those either substantially lacking or having enhanced collateral cleavage activity, such as Cas13f effector proteins based on any one of SEQ ID NOs: 3-4, or the above orthologs, homologs, derivatives, and functional fragments thereof, which fusions are with moieties such as localization signals, reporter genes (e.g., GST, HRP, CAT, GFP, HcRed, DsRed, CFP, YFP, BFP), NLS, protein targeting moieties, DNA binding domains (e.g., MBP, Lex A DBD, Gal4 DBD), epitope tags (e.g., His, myc, V5, FLAG, HA, VSV-G, Trx, etc), transcription activation domains (e.g., VP64 or VPR), transcription inhibition domains (e.g., KRAB moiety or SID moiety), nucleases (e.g., FokI), deaminase domain (e.g., ADARI, ADAR2, APOBEC, AID, or TAD), methylase domain, demethylase domain, transcription release factor, HDAC, a moiety having ssRNA cleavage activity, a moiety having dsRNA cleavage activity, a moiety having ssDNA cleavage activity, a moiety having dsDNA cleavage activity, DNA or RNA ligase domain, or any combination thereof.

For example, the fusion may include one or more NLSs, which can be located at or near N-terminal, C-terminal, internally, or combination thereof. In certain embodiments, conjugations do not affect the function of the original engineered Cas13f protein, such as those either substantially lacking or having enhanced collateral activity, such as the ability to bind a guide RNA/crRNA of the disclosure (described herein below) to form a complex, the RNase activity, and the ability to bind to and cleave a target RNA at a specific site, under the guidance of the crRNA that is at least partially complementary to the target RNA.

In another aspect, the disclosure provides a polynucleotide encoding the engineered Cas13f of the disclosure. The polynucleotide may comprise: (i) a polynucleotide encoding any one of the engineered Cas13f effector protein of the disclosure, such as those either substantially lacking or having enhanced collateral effect, e.g., those based on Cas13f effector proteins of SEQ ID NOs: 3-4, or orthologs, homologs, derivatives, functional fragments, fusions thereof; (ii) a polynucleotide comprising or encoding SEQ ID NO: 2; or (iii) a polynucleotide comprising (i) and (ii).

In certain embodiments, the polynucleotide of the disclosure is codon-optimized for expression in a eukaryote, a mammal (such as a human or a non-human mammal), a plant, an insect, a bird, a reptile, a rodent (e.g., mouse, rat), a fish, a worm/nematode, or a yeast.

In a related aspect, the disclosure provides a polynucleotide having (i) one or more (e.g., 1, 2, 3, 4, 5, 6, 7, 8, 9 or 10) nucleotides additions, deletions, or substitutions compared to the subject polynucleotide described above; (ii) at least 50%, 60%, 70%, 80%, 90%, 95%, or 97% sequence identity to the subject polynucleotide described above; (iii) hybridize under stringent conditions with the subject polynucleotide described above or any of (i) and (ii); or (iv) is a complement of any of (i)-(iii).

In another related aspect, the disclosure provides a vector comprising or encompassing any one of the polynucleotides of the disclosure described herein. The vector can be a cloning vector, or an expression vector. The vector can be a plasmid, phagemid, or cosmid, just to name a few. In certain embodiments, the vector can be used to express the polynucleotide in a mammalian cell, such as a human cell, any one of the engineered Cas13f, such as those either substantially lacking or having enhanced collateral activity, e.g., the subject engineered Cas13f effector proteins based on SEQ ID NOs: 3-4, or orthologs, homologs, derivatives, functional fragments, fusions thereof; or any of the polynucleotide of the disclosure; or any of the complex of the disclosure.

In certain embodiments, the polynucleotide is operably linked to a promoter and optionally an enhancer. For example, in some embodiments, the promoter is a constitutive promoter, an inducible promoter, a ubiquitous promoter, or a tissue specific promoter. In certain embodiments, the vector is a plasmid. In certain embodiments, the vector is a retroviral vector, a phage vector, an adenoviral vector, a herpes simplex viral (HSV) vector, an AAV vector, or a lentiviral vector. In certain embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, or AAV 13. In certain embodiments.

Another aspect of the disclosure provides a delivery system comprising (1) a delivery vehicle, and (2) the engineered Cas13f of the disclosure, the polynucleotide of the disclosure, or the vector of the disclosure.

In certain embodiments, the delivery vehicle is a nanoparticle, a liposome, an exosome, a microvesicle, or a gene-gun.

A further aspect of the disclosure provides a cell or a progeny thereof, comprising the engineered Cas13f of the disclosure, the polynucleotide of the disclosure, or the vector of the disclosure. The cell can be a prokaryote such as E. coli, or a cell from a eukaryote such as yeast, insect, plant, animal (e.g., mammal including human and mouse). The cell can be isolated primary cell (such as bone marrow cells for ex vivo therapy), or established cell lines such as tumor cell lines, 293T cells, or stem cells, iPCs, etc.

In certain embodiments, the cell or progeny thereof is a eukaryotic cell (e.g., a non-human mammalian cell, a human cell, or a plant cell) or a prokaryotic cell (e.g., a bacteria cell).

A further aspect of the disclosure provides a non-human multicellular eukaryote comprising the cell of the disclosure.

In certain embodiments, the non-human multicellular eukaryote is an animal (e.g., rodent or primate) model for a human genetic disorder.

In another aspect, the disclosure provides a complex comprising: (i) a protein composition of any one of the subject engineered Cas13f, such as those either substantially lacking or having enhanced collateral cleavage activity, e.g., engineered Cas13f effector protein, ororthologs, homologs, derivatives, conjugates, functional fragments thereof, conjugates thereof, or fusions thereof; and (ii) a polynucleotide composition, comprising an isolated polynucleotide comprising a cognate DR sequence for the engineered Cas13f effector protein, and a spacer/guide sequence complementary to at least a portion of a target RNA.

In certain embodiments, the DR sequence is at the 3′ end of the spacer sequence.

In certain embodiments, the DR sequence is at the 5′ end of the spacer sequence.

In some embodiments, the polynucleotide composition is the guide RNA/crRNA of the subject engineered Cas13f, such as those either substantially lacking or having enhanced collateral activity, e.g., engineered Cas13f system, which does not include a tracrRNA.

In certain embodiments, for use with the subject engineered Cas13f, such as those either substantially lacking or having enhanced collateral activity, e.g., the subject engineered Cas13f effector proteins, homologs, orthologs, derivatives, fusions, conjugates, or functional fragments thereof having guide sequence-specific RNase activity, the spacer sequence is at least about 10 nucleotides, or between 10-60, 15-50, 20-50, 25-40, 25-50, or 19-50 nucleotides.

In a related aspect, the disclosure provides a eukaryotic cell comprising a subject complex comprising a subject engineered Cas13f, the complex comprising: (1) an RNA guide sequence comprising a spacer sequence capable of hybridizing to a target RNA, and a direct repeat (DR) sequence 5′ or 3′ to the spacer sequence; and, (2) a subject engineered Cas13f, such as those either substantially lacking or having enhanced collateral activity, such as a subject engineered Cas13f effector protein based on a wild type having an amino acid sequence of any one of SEQ ID NOs: 3-4, or a derivative or functional fragment of the Cas; wherein the Cas, the derivative, and the functional fragment of the Cas, are capable of (i) binding to the RNA guide sequence and (ii) targeting the target RNA.

In another aspect, the disclosure provides a composition comprising: (i) a first (protein) composition selected from any one of the engineered Cas13f, such as those either substantially lacking or having enhanced collateral activity, e.g., engineered Cas13f effector proteins based on SEQ ID NOs: 3-4, or orthologs, homologs, derivatives, conjugates, functional fragments, fusions thereof; and (ii) a second (nucleotide) composition comprising an RNA encompassing a guide RNA/crRNA, particularly a spacer sequence, or a coding sequence for the same. The guide RNA may comprise a DR sequence, and a spacer sequence which can complement or hybridize with a target RNA. The guide RNA can form a complex with the first (protein) composition of (i). In some embodiment, the DR sequence can be the polynucleotide of the disclosure. In some embodiment, the DR sequence can be at the 5- or 3′-end of the guide RNA. In some embodiments, the composition (such as (i) and/or (ii)) is non-naturally occurring or modified from a naturally occurring composition. In some embodiments, the target sequence is an RNA from a prokaryote or a eukaryote, such as a non-naturally existing RNA. The target RNA may be present inside a cell, such as in the cytosol or inside an organelle. In some embodiments, the protein composition may have an NLS that can be located at its N- or C-terminal, or internally.

In another aspect, the disclosure provides a composition comprising one or more vectors of the disclosure, the one or more vectors comprise: (i) a first polynucleotide that encodes any one of the engineered Cas13f, such as those either substantially lacking or having enhanced collateral activity, such as a subject engineered Cas13f effector proteins based on SEQ ID NOs: 3-4, or orthologs, homologs, derivatives, functional fragments, fusions thereof; optionally operably linked to a first regulatory element; and (ii) a second polynucleotide that encodes a guide RNA of the disclosure; optionally operably linked to a second regulatory element. The first and the second polynucleotides can be on different vectors, or on the same vector. The guide RNA can form a complex with the protein product encoded by the first polynucleotide, and comprises a DR sequence (such as any one of the 4th aspect) and a spacer sequence that can bind to/complement with a target RNA. In some embodiments, the first regulatory element is a promoter, such as an inducible promoter. In some embodiments, the second regulatory element is a promoter, such as an inducible promoter. In some embodiments, the target sequence is an RNA from a prokaryote or a eukaryote, such as a non-naturally existing RNA.

The target RNA may be present inside a cell, such as in the cytosol or inside an organelle. In some embodiments, the protein composition may have an NLS that can be located at its N- or C-terminal, or internally.

In some embodiments, the vector is a plasmid. In some embodiment, the vector is a viral vector based on a retrovirus, a replication incompetent retrovirus, adenovirus, replication incompetent adenovirus, or AAV. In some embodiments, the vector can self-replicate in a host cell (e.g., having a bacterial replication origin sequence). In some embodiments, the vector can integrate into a host genome and be replicated therewith. In some embodiment, the vector is a cloning vector. In some embodiment, the vector is an expression vector.

The disclosure further provides a delivery composition for delivering any of the engineered Cas13f, such as those either substantially lacking or having enhanced collateral activity, e.g., a subject engineered Cas3f effector proteins based on SEQ ID NOs: 3-4, or orthologs, homologs, derivatives, conjugates, functional fragments, fusions thereof of the disclosure; the polynucleotide of the disclosure; the complex of the disclosure; the vector of the disclosure; the cell of the disclosure, and the composition of the disclosure. The delivery can be through any one known in the art, such as transfection, lipofection, electroporation, gene gun, microinjection, sonication, calcium phosphate transfection, cation transfection, viral vector delivery, etc., using vehicles such as liposome(s), nanoparticle(s), exosome(s), microvesicle(s), a gene-gun or one or more viral vector(s).

The disclosure further provides a kit comprising any one or more of the following: any of the engineered Cas13f, such as those either substantially lacking or having enhanced collateral activity, e.g., a subject engineered Cas3f effector proteins based on SEQ ID NOs: 3-4, or orthologs, homologs, derivatives, conjugates, functional fragments, fusions thereof of the disclosure; the polynucleotide of the disclosure; the complex of the disclosure; the vector of the disclosure; the cell of the disclosure, and the composition of the disclosure. In some embodiments, the kit may further comprise an instruction for how to use the kit components, and/or how to obtain additional components from 3^{rd} party for use with the kit components. Any component of the kit can be stored in any suitable container.

Another aspect of the disclosure provides an engineered Cas13f effector protein comprising any one or more mutations as described in any of the Examples, such as Example 1, 2, 4, 5, or 12.

In certain embodiments, the engineered Cas13f effector protein exhibits about the same or enhanced guide-RNA-mediated cleavage of a target RNA complementary to the guide RNA, as compared to that of the wild type Cas13f effector protein from which the engineered Cas13f effector protein derives (or theoretical maximum thereof).

In certain embodiments, the engineered Cas13f effector protein exhibits reduced or diminished guide-RNA independent or collateral cleavage of a non-specific RNA (e.g., one not substantially complementary to the guide RNA), as compared to that of the wild type Cas13f effector protein (or theoretical maximum thereof) from which the engineered Cas13f effector protein derives. For example, the engineered Cas13f effector protein exhibits about 50%, 40%, 30%, 20%, 15%, 10% or less collateral cleavage compared to that of the wild type Cas13f effector protein (or theoretical maximum thereof) from which the engineered Cas13f effector protein derives.

In certain embodiments, the engineered Cas13f effector protein exhibits increased guide-RNA independent or collateral cleavage of a non-specific RNA (e.g., one not substantially complementary to the guide RNA), as compared to that of the wild type Cas13f effector protein from which the engineered Cas13f effector protein derives. For example, the engineered Cas13f effector protein exhibits about 105%, 110%, 120%, 130%, 140%, 150%, 160%, 170%, 180%, 190%, 200% or more collateral cleavage compared to that of the wild type Cas13f effector protein from which the engineered Cas13f effector protein derives.

With the disclosures generally described herein above, more detailed descriptions for the various aspects of the disclosure are provided in separate sections below. However, it should be understood that, for simplicity and to reduce redundancy, certain embodiments of the disclosure are only described under one section or only described in the claims or examples. Thus it should also be understood that any one embodiment of the disclosure, including those described only under one aspect, section, or only in the claims or examples, can be combined with any other embodiment of the disclosure, unless specifically disclaimed or the combination is improper.

### 2. Representative Engineered Cas13f Polypeptide and Derivatives Thereof

One aspect of the disclosure provides engineered Cas13f effector protein, such as those either substantially lacking or having enhanced collateral activity.

As used herein, “(engineered) Cas13f”, “(engineered) Cas13f effector protein”, “(engineered) Cas13f effector enzyme”, “(engineered) Cas13f protein”, and “(engineered) Cas13f polypeptide” are exchangeable.

In certain embodiments, the Cas13f effector protein is a Cas13f effector protein having two strictly conserved RX4-6H (RXXXXH)-like motifs, characteristic of Higher Eukaryotes and Prokaryotes Nucleotide-binding (HEPN) domains. In certain embodiments, the Cas13f effector proteins that contain two HEPN domains have been previously characterized.

HEPN domains have been shown to be RNase domains and confer the ability to bind to and cleave target RNA molecule. The target RNA may be any suitable form of RNA, including but not limited to mRNA, tRNA, ribosomal RNA, non-coding RNA, lncRNA (long non-coding RNA), and nuclear RNA. For example, in some embodiments, the engineered Cas13f proteins recognize and cleave RNA targets located on the coding strand of open reading frames (ORFs).

Direct comparison of wild type Cas13f effector proteins with the effector protein of other CRISPR-Cas13 systems shows that Cas13f effector proteins are significantly smaller (e.g., about 20% fewer amino acids) than even the smallest previously identified Type VI-D/Cas13d effector proteins and have less than 30% sequence similarity in one-to-one sequence alignments to other previously described effector proteins, including the phylogenetically closest relatives Cas13b.

Cas13f proteins can be used in a variety of applications and are particularly suitable for therapeutic applications since they are significantly smaller than other effector proteins (e.g., CRISPR Cas13a, Cas13b, Cas13c, and Cas13d/CasRx effector proteins) which allows for the packaging of the nucleic acids encoding the effector proteins and their guide RNA coding sequences into delivery systems having size limitations, such as the AAV vectors. Further, the lack of detectable collateral/non-specific RNase activity of the subject engineered Cas13f, upon activation of the guide sequence-specific RNase activity, makes these engineered Cas13f effector proteins less prong to (if not immune from) potentially dangerous generalized off-target RNA digestion in target cells that are desirably not destroyed.

Exemplary Cas13f effector proteins include SEQ ID NO: 1 (Cas13f.1) of the disclosure, SEQ ID NOs: 2-7 (Cas13f2, Cas13f3, Cas13f4, and Cas13f5, respectively) of PCT/CN2020/077211, and SEQ ID NOs: 9-10 (Cas13f6 and Cas13f7, respectively) of PCT/CN2022/101884, such as SEQ ID NO: 1 of the disclosure, any of which may be taken as a reference Cas13f polypeptide.

In the sequences above, the two RX4-6H (RXXXXH) motifs in each effector are double-underlined. Mutations at one or both such domains may create an RNase dead version (or “dCas) of the Cas13f effector proteins, homologs, orthologs, fusions, conjugates, derivatives, or functional fragments thereof, while substantially maintaining their ability to bind the guide RNA and the target RNA complementary to the guide RNA.

The corresponding DR coding sequences for the Cas effector proteins are SEQ ID NO: 2 (Cas13f.1) of the disclosure, any one of SEQ ID NOs: 11-14 (Cas13f.2, Cas13f.3, Cas13f.4, and Cas13f.5, respectively) of PCT/CN2020/077211, incorporated herein by reference in its entirety, or any one of SEQ ID NOs: 26-27 (Cas13f.6 and Cas13f7, respectively) of PCT/CN2022/101884, incorporated herein by reference in its entirety, respectively.

In some embodiments, a subject engineered Cas13f effector protein, such as those either substantially lacking or having enhanced collateral activity is based on a “derivative” of a wild type Cas13f effector proteins, the derivative having an amino acid sequence with at least about 80% sequence identity to the amino acid sequence of any one of the wild type or reference Cas13f polypeptides herein (e.g., 81%, 82%, 83%, 84%, 85%, 86%, 87% 88%, 89%, 90%, 91%, 92%, 93%, 94%, 95%, 96%, 97%, 98%, 99%, or 100%). Such derivative Cas13f effector proteins sharing significant protein sequence identity to any one of the wild type or reference Cas13f polypeptides herein have retained at least one of the functions of the Cas of the corresponding wild type or reference Cas13f polypeptide herein (see below), such as the ability to bind to and form a complex with a crRNA comprising at least one of the DR sequences of Cas13f herein. For example, a Cas13f derivative may share 85% amino acid sequence identity to SEQ ID NO: 1, respectively, and retains the ability to bind to and form a complex with a crRNA having a DR sequence of SEQ ID NO: 2.

In certain embodiments, the sequence identity between the derivative and the wild type Cas13f is based on regions outside the regions defined by any one of the segments in Examples 1.

In some embodiments, the derivative comprises conserved amino acid residue substitutions. In some embodiments, the derivative comprises only conserved amino acid residue substitutions (i.e., all amino acid substitutions in the derivative are conserved substitutions, and there is no substitution that is not conserved).

In some embodiments, the derivative comprises no more than 1, 2, 3, 4, 5, 6, 7, 8, 9, or 10 amino acid insertions or deletions into any one of the sequences of the wild type or reference Cas13f polypeptides herein. The insertion and/or deletion maybe clustered together, or separated throughout the entire length of the sequences, so long as at least one of the functions of the wild type sequence is preserved. Such functions may include the ability to bind the guide/crRNA, the RNase activity, the ability to bind to and/or cleave the target RNA complementary to the guide/crRNA. In some embodiments, the insertions and/or deletions are not present in the RXXXXH motifs, or within 5, 10, 15, or 20 residues from the RXXXXH motifs.

In some embodiments, the derivative has retained the ability to bind guide RNA/crRNA.

In some embodiments, the derivative has retained the guide/crRNA-activated RNase activity.

In some embodiments, the derivative has retained the ability to bind target RNA and/or cleave the target RNA in the presence of the bound guide/crRNA that is complementary in sequence to at least a portion of the target RNA.

In other embodiments, the derivative has completely or partially lost the guide/crRNA-activated RNase activity, due to, for example, mutations in one or more catalytic residues of the RNA-guided RNase. Such derivatives are sometimes referred to as dCas13f.

Thus in certain embodiments, the derivative may be modified to have diminished nuclease/RNase activity, e.g., nuclease inactivation of at least 50%, at least 60%, at least 70%, at least 80%, at least 90%, at least 95%, at least 97%, or 100% as compared with the counterpart wild type proteins. The nuclease activity can be diminished by several methods known in the art, e.g., introducing mutations into the nuclease (catalytic) domains of the proteins. In some embodiments, catalytic residues for the nuclease activities are identified, and these amino acid residues can be substituted by different amino acid residues (e.g., glycine or alanine) to diminish the nuclease activity. In some embodiments, the amino acid substitution is a conservative amino acid substitution. In some embodiments, the amino acid substitution is a non-conservative amino acid substitution.

In some embodiments, the modification comprises one or more mutations (e.g., amino acid deletions, insertions, or substitutions) in at least one HEPN domain. In some embodiments, there is one, two, three, four, five, six, seven, eight, nine, or more amino acid substitutions in at least one HEPN domain. In certain embodiments, the one or more mutations or the two or more mutations may be in a catalytically active domain of the effector protein comprising a HEPN domain, or a catalytically active domain which is homologous to a HEPN domain.

The skilled person will understand that corresponding amino acid positions in different Cas13f proteins, such as different Cas13f proteins, may be mutated to the same effect. A multisequence alignment of several representative Cas13f family enzymes can be made by one of skill in the art. One of skill in the art can readily map the mutations in any Cas13f family protein sharing substantial sequence homology/identical to determine the mutations “corresponding to” the exemplified Cas13f mutations described herein.

In certain embodiments, one or more mutations abolishes catalytic activity of the protein completely or partially (e.g., altered cleavage rate, altered specificity, etc.).

The presence of at least one of these mutations results in a derivative having reduced or diminished guide sequence-dependent RNase activity as compared to the corresponding wild type protein lacking the mutations. The additional presence of any one of the mutations in the subject engineered Cas13f substantially lacking collateral effect can reduce/eliminate off-target effect resulting from non-specific RNA binding.

In certain embodiments, the effector protein as described herein is a “dead” effector protein, such as a dead Cas13f effector protein (i.e., dCas13f). In certain embodiments, the effector protein has one or more mutations in HEPN domain 1 (N-terminal). In certain embodiments, the effector protein has one or more mutations in HEPN domain 2 (C-terminal). In certain embodiments, the effector protein has one or more mutations in HEPN domain 1 and HEPN domain 2.

In some embodiment, the dCas13f is a Cas13f mutant with R77A, H82A, R764A, and H769A mutations based on the reference Cas13f polypeptide of SEQ ID NO: 1.

The inactivated Cas or derivative or functional fragment thereof can be fused or associated with one or more heterologous/functional domains (e.g., via fusion protein, linker peptides, “GS” linkers, etc.).

These functional domains can have various activities, e.g., methylase activity, demethylase activity, transcription activation activity, transcription repression activity, transcription release factor activity, histone modification activity, RNA cleavage activity, DNA cleavage activity, nucleic acid binding activity, base-editing activity, and switch activity (e.g., light inducible). In some embodiments, the functional domains are Krüppel associated box (KRAB), SID (e.g. SID4X), VP64, VPR, VP16, Fok1, P65, HSF1, MyoD1, Adenosine Deaminase Acting on RNA such as ADAR1, ADAR2, APOBEC, cytidine deaminase (AID), TAD, mini-SOG, APEX, and biotin-APEX.

In some embodiments, the functional domain is a base editing domain, e.g., ADAR1 (including wild type or ADAR2DD version thereof, with or without the E1008Q and/or the E488Q mutation(s)), ADAR2 (including wild type or ADAR2DD version thereof, with or without the E1008Q and/or the E488Q mutation(s)), APOBEC, or AID.

In some embodiments, the functional domain may comprise one or more nuclear localization signal (NLS) domains. The one or more heterologous functional domains may comprise at least two or more NLS domains. The one or more NLS domain(s) may be positioned at or near or in proximity to a terminus of the effector protein (e.g., Cas13f effector proteins) and if two or more NLSs, each of the two may be positioned at or near or in proximity to a terminus of the effector protein (e.g., Cas13f effector proteins).

In some embodiments, at least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In some embodiments, multiple (e.g., two, three, four, five, six, seven, eight, or more) identical or different functional domains are present.

In some embodiments, the functional domain (e.g., a base editing domain) is further fused to an RNA-binding domain (e.g., MS2).

In some embodiments, the functional domain is associated to or fused via a linker sequence (e.g., a flexible linker sequence or a rigid linker sequence). Exemplary linker sequences and functional domain sequences are provided in PCT/CN2021/121926.

The positioning of the one or more functional domains on the inactivated Cas proteins is one that allows for correct spatial orientation for the functional domain to affect the target with the attributed functional effect. For example, if the functional domain is a transcription activator (e.g., VP16, VP64, or p65), the transcription activator is placed in a spatial orientation that allows it to affect the transcription of the target. Likewise, a transcription repressor is positioned to affect the transcription of the target, and a nuclease (e.g., Fok1) is positioned to cleave or partially cleave the target. In some embodiments, the functional domain is positioned at the N-terminus of the Cas/dCas. In some embodiments, the functional domain is positioned at the C-terminus of the Cas/dCas. In some embodiments, the inactivated CRISPR-associated protein (dCas) is modified to comprise a first functional domain at the N-terminus and a second functional domain at the C-terminus.

Various examples of inactivated CRISPR-associated proteins fused with one or more functional domains and methods of using the same are described, e.g., in International Publication No. WO 2017/219027, which is incorporated herein by reference in its entirety, and in particular with respect to the features described herein.

In some embodiments, instead of using full-length wild type or derivative Cas13f effector proteins, “functional fragments” thereof can be used.

A “functional fragment,” as used herein, refers to a fragment of a wild type Cas13f protein or a derivative thereof, that has less-than full-length sequence. The deleted residues in the functional fragment can be at the N-terminus, the C-terminus, and/or internally. The functional fragment retains at least one function of the wild type Cas13f, or at least one function of its derivative. Thus a functional fragment is defined specifically with respect to the function at issue. For example, a functional fragment, wherein the function is the ability to bind crRNA and target RNA, may not be a functional fragment with respect to the RNase function, because losing the RXXXXH motifs at both ends of the Cas may not affect its ability to bind a crRNA and target RNA, but may eliminate/destroy the RNase activity.

In certain embodiments, the engineered Cas13f of the disclosure including a functional fragment of an engineered Cas13f that substantially retains the corresponding wild type Cas13fs guide sequence-dependent RNase activity, but substantially lacks collateral activity.

In some embodiments, compared to full-length wild type sequences, the engineered Cas13f effector proteins or derivatives thereof or functional fragments thereof lacks about 30, 60, 90, 120, 150, or about 180 residues from the N-terminus.

In some embodiments, compared to full-length wild type sequences, the engineered Cas13f effector proteins or derivatives thereof or functional fragments thereof lacks about 30, 60, 90, 120, or about 150 residues from the C-terminus.

In some embodiments, compared to full-length wild type sequences, the engineered Cas13f effector proteins or derivatives thereof or functional fragments thereof lacks about 30, 60, 90, 120, 150, or about 180 residues from the N-terminus, and lacks about 30, 60, 90, 120, or about 150 residues from the C-terminus.

In some embodiments, the engineered Cas13f effector proteins or derivatives thereof or functional fragments thereof have RNase activity, e.g., guide/crRNA-activated specific RNase activity.

In some embodiments, the engineered Cas13f effector proteins or derivatives thereof or functional fragments thereof have no substantial/detectable collateral RNase activity.

The present disclosure also provides a split version of the engineered Cas13f effector protein described herein. The split version of the engineered Cas13f may be advantageous for delivery. In some embodiments, the engineered Cas13f is split into two parts of the enzyme, which together substantially comprise a functioning engineered Cas13f.

The split can be done in a way that the catalytic domain(s) are unaffected. The CRISPR-associated protein may function as a nuclease or may be an inactivated enzyme, which is essentially a RNA-binding protein with very little or no catalytic activity (e.g., due to mutation(s) in its catalytic domains).

Split enzymes are described, e.g., in Wright et al., “Rational design of a split-Cas9 enzyme complex,” Proc. Nat'l. Acad. Sci. 112(10): 2984-2989, 2015, which is incorporated herein by reference in its entirety.

For example, in some embodiments, the nuclease lobe and α-helical lobe are expressed as separate polypeptides. Although the lobes do not interact on their own, the crRNA recruits them into a ternary complex that recapitulates the activity of full-length CRISPR-associated proteins and catalyzes site-specific cleavage. The use of a modified crRNA abrogates split-enzyme activity by preventing dimerization, allowing for the development of an inducible dimerization system.

In some embodiments, the split CRISPR-associated protein can be fused to a dimerization partner, e.g., by employing rapamycin sensitive dimerization domains. This allows the generation of a chemically inducible CRISPR-associated protein for temporal control of the activity of the protein. The CRISPR-associated protein can thus be rendered chemically inducible by being split into two fragments and rapamycin-sensitive dimerization domains can be used for controlled re-assembly of the protein.

The split point is typically designed in silico and cloned into the constructs. During this process, mutations can be introduced to the split CRISPR-associated protein and non-functional domains can be removed.

In some embodiments, the two parts or fragments of the split CRISPR-associated protein (i.e., the N-terminal and C-terminal fragments), can form a full CRISPR-associated protein, comprising, e.g., at least 70%, at least 80%, at least 90%, at least 95%, or at least 99% of the sequence of the wild type CRISPR-associated protein.

The Cas13f effector proteins described herein can be designed to be self-activating or self-inactivating. For example, the target sequence can be introduced into the coding construct of the CRISPR-associated protein. Thus, the CRISPR-associated protein can cleave the target sequence, as well as the construct encoding the protein thereby self-inactivating their expression. Methods of constructing a self-inactivating CRISPR system are described, e.g., in Epstein and Schaffer, Mol. Ther 24: S50, 2016, which is incorporated herein by reference in its entirety.

In some other embodiments, an additional crRNA, expressed under the control of a weak promoter (e.g., 7SK promoter), can target the nucleic acid sequence encoding the CRISPR-associated protein to prevent and/or block its expression (e.g., by preventing the transcription and/or translation of the nucleic acid). The transfection of cells with vectors expressing the CRISPR-associated protein, the crRNAs, and crRNAs that target the nucleic acid encoding the CRISPR-associated protein can lead to efficient disruption of the nucleic acid encoding the CRISPR-associated protein and decrease the levels of CRISPR-associated protein, thereby limiting its activity.

In some embodiments, the activity of the CRISPR-associated protein can be modulated through endogenous RNA signatures (e.g., miRNA) in mammalian cells. A CRISPR-associated protein switch can be made by using a miRNA-complementary sequence in the 5′-UTR of mRNA encoding the CRISPR-associated protein. The switches selectively and efficiently respond to miRNA in the target cells. Thus, the switches can differentially control the Cas activity by sensing endogenous miRNA activities within a heterogeneous cell population. Therefore, the switch systems can provide a framework for cell-type selective activity and cell engineering based on intracellular miRNA information (see, e.g., Hirosawa et al., Nucl. Acids Res. 45(13): e118, 2017).

The engineered Cas13f effector proteins, such as those either substantially lacking or having enhanced collateral activity can be inducibly expressed, e.g., their expression can be light-induced or chemically-induced. This mechanism allows for activation of the functional domain in the CRISPR-associated proteins. Light inducibility can be achieved by various methods known in the art, e.g., by designing a fusion complex wherein CRY2 PHR/CIBN pairing is used in split CRISPR-associated proteins (see, e.g., Konermann et al., “Optical control of mammalian endogenous transcription and epigenetic states,” Nature 500:7463, 2013.

Chemical inducibility can be achieved, e.g., by designing a fusion complex wherein FKBP/FRB (FK506 binding protein/FKBP rapamycin binding domain) pairing is used in split CRISPR-associated proteins. Rapamycin is required for forming the fusion complex, thereby activating the CRISPR-associated proteins (see, e.g., Zetsche et al., “A split-Cas9 architecture for inducible genome editing and transcription modulation,” Nature Biotech. 33:2:139-42, 2015).

Furthermore, expression of the engineered Cas13f effector proteins, such as those either substantially lacking or having enhanced collateral activity can be modulated by inducible promoters, e.g., tetracycline or doxycycline controlled transcriptional activation (Tet-On and Tet-Off expression system), hormone inducible gene expression system (e.g., an ecdysone inducible gene expression system), and an arabinose-inducible gene expression system. When delivered as RNA, expression of the RNA targeting effector protein can be modulated via a riboswitch, which can sense a small molecule like tetracycline (see, e.g., Goldfless et al., “Direct and specific chemical control of eukaryotic translation with a synthetic RNA-protein interaction,” Nucl. Acids Res. 40:9: e64-e64, 2012).

Various embodiments of inducible CRISPR-associated proteins and inducible CRISPR systems are described, e.g., in U.S. Pat. No. 8,871,445, US Publication No. 2016/0208243, and International Publication No. WO 2016/205764, each of which is incorporated herein by reference in its entirety.

In some embodiments, the engineered Cas13f effector proteins, such as those either substantially lacking or having enhanced collateral activity include at least one (e.g., 1, 2, 3, 4, 5, 6, 7, 8, 9, or 10) Nuclear Localization Signal (NLS) attached to the N-terminal or C-terminal of the protein. Non-limiting examples of NLSs include an NLS sequence derived from: the NLS of the SV40 virus large T-antigen, having the amino acid sequence of SEQ ID NO: 5; the NLS from nucleoplasmin (e.g., the nucleoplasmin bipartite NLS); c-myc NLS; hRNPA1 M9 NLS; IBB domain from importin-alpha; myoma T protein; human p53; mouse c-abl IV; influenza virus NS1; Hepatitis virus delta antigen; mouse Mx1 protein; human poly(ADP-ribose) polymerase; and human glucocorticoid receptor. In some embodiments, the CRISPR-associated protein comprises at least one (e.g., 1, 2, 3, 4, 5, 6, 7, 8, 9, or 10) Nuclear Export Signal (NES) attached the N-terminal or C-terminal of the protein. In a preferred embodiment a C-terminal and/or N-terminal NLS or NES is attached for optimal expression and nuclear targeting in eukaryotic cells, e.g., human cells.

In some embodiments, the engineered Cas13f effector proteins, such as those either substantially lacking or having enhanced collateral activity are mutated at one or more amino acid residues to alter one or more functional activities.

For example, in some embodiments, the engineered Cas13f effector proteins, such as those either substantially lacking or having enhanced collateral activity is mutated at one or more amino acid residues to alter its helicase activity.

In some embodiments, the engineered Cas13f effector proteins, such as those either substantially lacking or having enhanced collateral activity is mutated at one or more amino acid residues to alter its nuclease activity (e.g., endonuclease activity or exonuclease activity), such as the collateral nuclease activity that is not dependent on guide sequence.

In some embodiments, the engineered Cas13f effector proteins, such as those either substantially lacking or having enhanced collateral activity is mutated at one or more amino acid residues to alter its ability to functionally associate with a guide RNA.

In some embodiments, the engineered Cas13f effector proteins, such as those either substantially lacking or having enhanced collateral activity is mutated at one or more amino acid residues to alter its ability to functionally associate with a target nucleic acid.

In some embodiments, the engineered Cas13f effector proteins, such as those either substantially lacking or having enhanced collateral activity described herein are capable of cleaving a target RNA molecule.

In some embodiments, the engineered Cas13f effector proteins, such as those either substantially lacking or having enhanced collateral activity is mutated at one or more amino acid residues to alter its cleaving activity. For example, in some embodiments, the engineered Cas13f effector proteins, such as those either substantially lacking or having enhanced collateral activity may comprise one or more mutations that render the enzyme incapable of cleaving a target nucleic acid.

In some embodiments, the engineered Cas13f effector proteins, such as those either substantially lacking or having enhanced collateral activity is capable of cleaving the strand of the target nucleic acid that is complementary to the strand to which the guide RNA hybridizes.

In some embodiments, a engineered Cas13f effector proteins, such as those either substantially lacking or having enhanced collateral activity described herein can be engineered to have a deletion in one or more amino acid residues to reduce the size of the enzyme while retaining one or more desired functional activities (e.g., nuclease activity and the ability to interact functionally with a guide RNA). The truncated engineered Cas13f effector proteins, such as those either substantially lacking or having enhanced collateral activity can be advantageously used in combination with delivery systems having load limitations.

In some embodiments, the engineered Cas13f effector proteins, such as those either substantially lacking or having enhanced collateral activity described herein can be fused to one or more peptide tags, including a His-tag, GST-tag, a V5-tag, FLAG-tag, HA-tag, VSV-G-tag, Trx-tag, or myc-tag.

In some embodiments, the engineered Cas13f effector proteins, such as those either substantially lacking or having enhanced collateral activity described herein can be fused to a detectable moiety such as GST, a fluorescent protein (e.g., GFP, HcRed, DsRed, CFP, YFP, or BFP), or an enzyme (such as HRP or CAT).

In some embodiments, the engineered Cas13f effector proteins, such as those either substantially lacking or having enhanced collateral activity described herein can be fused to MBP, LexA DNA binding domain, or Gal4 DNA-binding domain.

In some embodiments, the engineered Cas13f effector proteins, such as those either substantially lacking or having enhanced collateral activity described herein can be linked to or conjugated with a detectable label such as a fluorescent dye, including FITC and DAPI.

In any of the embodiments herein, the linkage between the engineered Cas13f effector proteins, such as those either substantially lacking or having enhanced collateral activity described herein and the other moiety can be at the N- or C-terminal of the CRISPR-associated proteins, and sometimes even internally via covalent chemical bonds. The linkage can be affected by any chemical linkage known in the art, such as peptide linkage, linkage through the side chain of amino acids such as D, E, S, T, or amino acid derivatives (Ahx, p-Ala, GABA or Ava), or PEG linkage.

### 3. Polynucleotides

The disclosure also provides nucleic acids encoding the proteins described herein (e.g., an engineered Cas13f protein, such as those either substantially lacking or having enhanced collateral activity).

In some embodiments, the nucleic acid is a synthetic nucleic acid. In some embodiments, the nucleic acid is a DNA molecule. In some embodiments, the nucleic acid is an RNA molecule (e.g., an mRNA molecule encoding the engineered Cas13f protein, such as those either substantially lacking or having enhanced collateral activity, derivative or functional fragment thereof). In some embodiments, the mRNA is capped, polyadenylated, substituted with 5-methyl cytidine, substituted with pseudouridine, or a combination thereof.

In some embodiments, the nucleic acid (e.g., DNA) is operably linked to a regulatory element (e.g., a promoter) in order to control the expression of the nucleic acid. In some embodiments, the promoter is a constitutive promoter. In some embodiments, the promoter is an inducible promoter. In some embodiments, the promoter is a cell-specific promoter. In some embodiments, the promoter is an organism-specific promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, and a R-actin promoter. For example, a U6 promoter can be used to regulate the expression of a guide RNA molecule described herein.

In some embodiments, the nucleic acid(s) are present in a vector (e.g., a viral vector or a phage). The vector can be a cloning vector, or an expression vector. The vectors can be plasmids, phagemids, Cosmids, etc. The vectors may include one or more regulatory elements that allow for the propagation of the vector in a cell of interest (e.g., a bacterial cell or a mammalian cell). In some embodiments, the vector includes a nucleic acid encoding a single component of a CRISPR-associated (Cas) system described herein. In some embodiments, the vector includes multiple nucleic acids, each encoding a component of a CRISPR-associated (Cas) system described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding the engineered Cas13f protein substantially lacking collateral activity, derivatives, functional fragments, or guide/crRNA, including the DR sequences.

In another aspect, the present disclosure also provides nucleic acid sequences encoding amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences of the subject engineered Cas13f protein substantially lacking collateral activity.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the disclosure provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The proteins described herein (e.g., an engineered Cas13f protein substantially lacking collateral activity) can be delivered or used as either nucleic acid molecules or polypeptides.

In certain embodiments, the nucleic acid molecule encoding the engineered Cas13f protein, such as those either substantially lacking or having enhanced collateral activity, derivatives or functional fragments thereof are codon-optimized for expression in a host cell or organism. The host cell may include established cell lines (such as 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.oijp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

4. RNA Guides or crRNA

As used herein, the terms “guide sequence” and “spacer sequence” are exchangeable.

As used herein, the terms “RNA guide”, “crRNA”, “guide RNA”, and “gRNA” are exchangeable.

In some embodiments, the CRISPR systems described herein include at least RNA guide (e.g., a gRNA or a crRNA).

The architecture of multiple RNA guides is known in the art (see, e.g., International Publication Nos. WO 2014/093622 and WO 2015/070083, the entire contents of each of which are incorporated herein by reference).

In some embodiments, the CRISPR systems described herein include multiple RNA guides (e.g., one, two, three, four, five, six, seven, eight, or more RNA guides).

In some embodiments, the RNA guide includes a crRNA. In some embodiments, the RNA guide includes a crRNA but not a tracrRNA.

Sequences for guide RNAs from multiple CRISPR systems are generally known in the art, see, for example, Grissa et al. (Nucleic Acids Res. 35 (web server issue): W52-7, 2007; Grissa et al., BMC Bioinformatics 8:172, 2007; Grissa et al., Nucleic Acids Res. 36 (web server issue): W145-8, 2008; and Moller and Liang, PeerJ 5: e3788, 2017; the CRISPR database at: crispr.i2bc.paris-saclayfr/crispr/BLAST/CRISPRsBlast.php; and MetaCRAST available at: github.com/molleraj/MetaCRAST). All incorporated herein by reference.

In some embodiments, the crRNA includes a direct repeat (DR) sequence and a spacer sequence. In certain embodiments, the crRNA comprises, consists essentially of, or consists of a direct repeat sequence linked to a guide sequence or spacer sequence, preferably at the 3′-end of the spacer sequence. In general, an engineered Cas13f protein, such as those either substantially lacking or having enhanced collateral activity forms a complex with the mature crRNA, which spacer sequence directs the complex to a sequence-specific binding with the target RNA that is complementary to the spacer sequence, and/or hybridizes to the spacer sequence. The resulting complex comprises the engineered Cas13f protein, such as those either substantially lacking or having enhanced collateral activity and the mature crRNA bound to the target RNA.

The direct repeat sequences for the Cas13f systems are generally well conserved, especially at the ends, with, for example, a GCTGT for Cas13f at the 5′-end, reverse complementary to a ACAGC for Cas13f at the 3′ end. This conservation suggests strong base pairing for an RNA stem-loop structure that potentially interacts with the protein(s) in the locus.

In some embodiments, the direct repeat sequence, when in RNA, comprises the general secondary structure of 5′-Sla-Ba-S2a-L-S2b-Bb-S1b-3′, wherein segments S1a and S1b are reverse complement sequences and form a first stem (S1) having 5 nucleotides in Cas13f; segments Ba and Bb do not base pair with each other and form a symmetrical or nearly symmetrical bulge (B), and have 5 (Ba) and 4 (Bb) or 6 (Ba) and 5 (Bb) nucleotides respectively in Cas13f; segments S2a and S2b are reverse complement sequences and form a second stem (S2) having either 6 or 5 base pairs in Cas13f; and L is a 5-nucleotide loop in Cas13f.

In certain embodiments, Sla has a sequence of GCUGU in Cas13f.

In certain embodiments, S2a has a sequence of A/G CCUC G/A in Cas13f (wherein the first A or G may be absent).

In some embodiments, the direct repeat sequence comprises, consists essentially of, or consists of a nucleic acid sequence of SEQ ID NO: 2.

As used herein, “direct repeat sequence” may refer to the DNA coding sequence in the CRISPR locus, or to the RNA encoded by the same in crRNA. Thus when SEQ ID NO: 2 is referred to in the context of an RNA molecule, such as crRNA, each T is understood to represent a U.

In some embodiments, the direct repeat sequence comprises, consists essentially of, or consists of a nucleic acid sequence having up to 1, 2, 3, 4, 5, 6, 7, or 8 nucleotides of deletion, insertion, or substitution of SEQ ID NO: 2. In some embodiments, the direct repeat sequence comprises, consists essentially of, or consists of a nucleic acid sequence having at least 80%, 85%, 90%, 95%, or 97% of sequence identity with SEQ ID NO: 2 (e.g., due to deletion, insertion, or substitution of nucleotides in SEQ ID NO: 2). In some embodiments, the direct repeat sequence comprises, consists essentially of, or consists of a nucleic acid sequence that is not identical to any one of SEQ ID NO: 2, but can hybridize with a complement of any one of SEQ ID NO: 2 under stringent hybridization conditions, or can bind to a complement of any one of SEQ ID NO: 2 under physiological conditions.

In certain embodiments, the deletion, insertion, or substitution does not change the overall secondary structure of that of SEQ ID NO: 2 (e.g., the relative locations and/or sizes of the stems and bulges and loop do not significantly deviate from that of the original stems, bulges, and loop). For example, the deletion, insert, or substitution may be in the bulge or loop region so that the overall symmetry of the bulge remains largely the same. The deletion, insertion, or substitution may be in the stems so that the length of the stems do not significantly deviate from that of the original stems (e.g., adding or deleting one base pair in each of the two stems correspond to 4 total base changes).

In certain embodiments, the deletion, insertion, or substitution results in a derivative DR sequence that may have ±1 or 2 base pair(s) in one or both stems, have ±1, 2, or 3 bases in either or both of the single strands in the bulge, and/or have ±1, 2, 3, or 4 bases in the loop region.

In certain embodiments, any of the above direct repeat sequences that is different from any one of SEQ ID NO: 2 retains the ability to function as a direct repeat sequence in the Cas13f proteins, as the DR sequence of SEQ ID NO: 2.

In some embodiments, the direct repeat sequence comprises, consists essentially of, or consists of a nucleic acid having a nucleic acid sequence of any one of SEQ ID NO: 2, with a truncation of the initial three, four, five, six, seven, or eight 3′ nucleotides.

In classic CRISPR systems, the degree of complementarity between a guide sequence (e.g., a crRNA) and its corresponding target sequence can be about 50%, 60%, 75%, 80%, 85%, 90%, 95%, 97.5%, 99%, or 100%. In some embodiments, the degree of complementarity is 90-100%.

The guide RNAs can be about 5, 10, 11, 12, 13, 14, 15, 16, 17, 18, 19, 20, 21, 22, 23, 24, 25, 26, 27, 28, 29, 30, 35, 40, 45, 50, 75, 100, 125, 150, 175, 200 or more nucleotides in length. For example, for use in a functional engineered Cas13f effector protein, or homologs, orthologs, derivatives, fusions, conjugates, or functional fragment thereof, the spacer can be between 10-60 nucleotides, 20-50 nucleotides, 25-45 nucleotides, 25-35 nucleotides, or about 27, 28, 29, 30, 31, 32, or 33 nucleotides. For use in dCas version of any of the above, however, the spacer can be between 10-200 nucleotides, 20-150 nucleotides, 25-100 nucleotides, 25-85 nucleotides, 35-75 nucleotides, 45-60 nucleotides, or about 46, 47, 48, 49, 50, 51, 52, 53, 54, or 55 nucleotides.

To reduce off-target interactions, e.g., to reduce the guide interacting with a target sequence having low complementarity, mutations can be introduced to the CRISPR systems so that the CRISPR systems can distinguish between target and off-target sequences that have greater than 80%, 85%, 90%, or 95% complementarity. In some embodiments, the degree of complementarity is from 80% to 95%, e.g., about 83%, 84%, 85%, 86%, 87%, 88%, 89%, 90%, 91%, 92%, 93%, 94%, or 95% (for example, distinguishing between a target having 18 nucleotides from an off-target of 18 nucleotides having 1, 2, or 3 mismatches). Accordingly, in some embodiments, the degree of complementarity between a guide sequence and its corresponding target sequence is greater than 94.5%, 95%, 95.5%, 96%, 96.5%, 97%, 97.5%, 98%, 98.5%, 99%, 99.5%, or 99.9%. In some embodiments, the degree of complementarity is 100%.

It is known in the field that complete complementarity is not required, provided there is sufficient complementarity to be functional. Modulations of cleavage efficiency can be exploited by introduction of mismatches, e.g., one or more mismatches, such as 1 or 2 mismatches between spacer sequence and target sequence, including the position of the mismatch along the spacer/target. The more central (i.e., not at the 3′ or 5′-ends) a mismatch, e.g., a double mismatch, is located; the more cleavage efficiency is affected. Accordingly, by choosing mismatch positions along the spacer sequence, cleavage efficiency can be modulated. For example, if less than 100% cleavage of targets is desired (e.g., in a cell population), 1 or 2 mismatches between spacer and target sequence can be introduced in the spacer sequences.

Type VI CRISPR-Cas effector proteins have been demonstrated to employ more than one RNA guide, thus enabling the ability of these effector proteins, and systems and complexes that include them, to target multiple nucleic acids. In some embodiments, the CRISPR systems comprising the engineered Cas13f protein, such as those either substantially lacking or having enhanced collateral activity, as described herein, include multiple RNA guides (e.g., two, three, four, five, six, seven, eight, nine, ten, fifteen, twenty, thirty, forty, or more) RNA guides. In some embodiments, the CRISPR systems described herein include a single RNA strand or a nucleic acid encoding a single RNA strand, wherein the RNA guides are arranged in tandem. The single RNA strand can include multiple copies of the same RNA guide, multiple copies of distinct RNA guides, or combinations thereof. The processing capability of the Cas13f effector proteins described herein enables these effector proteins to be able to target multiple target nucleic acids (e.g., target RNAs) without a loss of activity. In some embodiments, the Cas13f effector proteins may be delivered in complex with multiple RNA guides directed to different target RNA. In some embodiments, the engineered Cas13f protein, such as those either substantially lacking or having enhanced collateral activity may be co-delivered with multiple RNA guides, each specific for a different target nucleic acid. Methods of multiplexing using CRISPR-associated proteins are described, for example, in U.S. Pat. No. 9,790,490 B2, and EP 3009511 B1, the entire contents of each of which are expressly incorporated herein by reference.

The spacer length of crRNAs can range from about 10-50 nucleotides, such as 15-50 nucleotides, 20-50 nucleotides, 25-50 nucleotide, or 19-50 nucleotides. In some embodiments, the spacer length of a guide RNA is at least 16 nucleotides, at least 17 nucleotides, at least 18 nucleotides, at least 19 nucleotides, at least 20 nucleotides, at least 21 nucleotides, or at least 22 nucleotides. In some embodiments, the spacer length is from 15 to 17 nucleotides (e.g., 15, 16, or 17 nucleotides), from 17 to 20 nucleotides (e.g., 17, 18, 19, or 20 nucleotides), from 20 to 24 nucleotides (e.g., 20, 21, 22, 23, or 24 nucleotides), from 23 to 25 nucleotides (e.g., 23, 24, or 25 nucleotides), from 24 to 27 nucleotides, from 27 to 30 nucleotides, from 30 to 45 nucleotides (e.g., 30, 31, 32, 33, 34, 35, 36, 37, 38, 39, 40, 41, 42, 43, 44, or 45 nucleotides), from 30 or 35 to 40 nucleotides, from 41 to 45 nucleotides, from 45 to 50 nucleotides (e.g., 45, 46, 47, 48, 49, or 50 nucleotides), or longer. In some embodiments, the spacer length is from about 15 to about 42 nucleotides. In some embodiments, the spacer length is about 30 nucleotides.

In some embodiments, the direct repeat length of the guide RNA is 15-36 nucleotides, is at least 16 nucleotides, is from 16 to 20 nucleotides (e.g., 16, 17, 18, 19, or 20 nucleotides), is from 20-30 nucleotides (e.g., 20, 21, 22, 23, 24, 25, 26, 27, 28, 29, or 30 nucleotides), is from 30-40 nucleotides (e.g., 30, 31, 32, 33, 34, 35, 36, 37, 38, 39, or 40 nucleotides), or is about 36 nucleotides (e.g., 33, 34, 35, 36, 37, 38, or 39 nucleotides). In some embodiments, the direct repeat length of the guide RNA is 36 nucleotides.

In some embodiments, the overall length of the crRNA/guide RNA is about 36 nucleotides longer than any one of the spacer sequence length described herein above. For example, the overall length of the crRNA/guide RNA may be between 45-86 nucleotides, or 60-86 nucleotides, 62-86 nucleotides, or 63-86 nucleotides.

The crRNA sequences can be modified in a manner that allows for formation of a complex between the crRNA and the engineered Cas13f protein, such as those either substantially lacking or having enhanced collateral activity, and successful binding to the target, while at the same time not allowing for successful nuclease activity (i.e., without nuclease activity/without causing indels). These modified guide sequences are referred to as “dead crRNAs,” “dead guides,” or “dead guide sequences.” These dead guides or dead guide sequences may be catalytically inactive or conformationally inactive with regard to nuclease activity. Dead guide sequences are typically shorter than respective guide sequences that result in active RNA cleavage. In some embodiments, dead guides are 5%, 10%, 20%, 30%, 40%, or 50%, shorter than respective guide RNAs that have nuclease activity. Dead guide sequences of guide RNAs can be from 13 to 15 nucleotides in length (e.g., 13, 14, or 15 nucleotides in length), from 15 to 19 nucleotides in length, or from 17 to 18 nucleotides in length (e.g., 17 nucleotides in length).

Thus, in one aspect, the disclosure provides non-naturally occurring or engineered CRISPR systems including a functional engineered Cas13f protein, such as those either substantially lacking or having enhanced collateral activity as described herein, and a crRNA, wherein the crRNA comprises a dead crRNA sequence whereby the crRNA is capable of hybridizing to a target sequence such that the CRISPR system is directed to a target RNA of interest in a cell without detectable nuclease activity (e.g., RNase activity).

A detailed description of dead guides is described, e.g., in International Publication No. WO 2016/094872, which is incorporated herein by reference in its entirety.

Guide RNAs (e.g., crRNAs) can be generated as components of inducible systems. The inducible nature of the systems allows for spatio-temporal control of gene editing or gene expression. In some embodiments, the stimuli for the inducible systems include, e.g., electromagnetic radiation, sound energy, chemical energy, and/or thermal energy.

In some embodiments, the transcription of guide RNA (e.g., crRNA) can be modulated by inducible promoters, e.g., tetracycline or doxycycline controlled transcriptional activation (Tet-On and Tet-Off expression systems), hormone inducible gene expression systems (e.g., ecdysone inducible gene expression systems), and arabinose-inducible gene expression systems. Other examples of inducible systems include, e.g., small molecule two-hybrid transcription activations systems (FKBP, ABA, etc.), light inducible systems (Phytochrome, LOV domains, or cryptochrome), or Light Inducible Transcriptional Effector (LITE). These inducible systems are described, e.g., in WO 2016205764 and U.S. Pat. No. 8,795,965, both of which are incorporated herein by reference in the entirety.

Chemical modifications can be applied to the crRNA's phosphate backbone, sugar, and/or base. Backbone modifications such as phosphorothioates modify the charge on the phosphate backbone and aid in the delivery and nuclease resistance of the oligonucleotide (see, e.g., Eckstein, “Phosphorothioates, essential components of therapeutic oligonucleotides,” Nucl. Acid Ther, 24, pp. 374-387, 2014); modifications of sugars, such as 2′-O-methyl (2′-OMe), 2′-F, and locked nucleic acid (LNA), enhance both base pairing and nuclease resistance (see, e.g., Allerson et al. “Fully 2′-modified oligonucleotide duplexes with improved in vitro potency and stability compared to unmodified small interfering RNA,” J. Med. Chem. 48.4: 901-904, 2005). Chemically modified bases such as 2-thiouridine or N6-methyladenosine, among others, can allow for either stronger or weaker base pairing (see, e.g., Bramsen et al., “Development of therapeutic-grade small interfering RNAs by chemical engineering,” Front. Genet., 2012 Aug. 20; 3:154). Additionally, RNA is amenable to both 5′ and 3′ end conjugations with a variety of functional moieties including fluorescent dyes, polyethylene glycol, or proteins.

A wide variety of modifications can be applied to chemically synthesized crRNA molecules. For example, modifying an oligonucleotide with a 2′-OMe to improve nuclease resistance can change the binding energy of Watson-Crick base pairing. Furthermore, a 2′-OMe modification can affect how the oligonucleotide interacts with transfection reagents, proteins or any other molecules in the cell. The effects of these modifications can be determined by empirical testing.

In some embodiments, the crRNA includes one or more phosphorothioate modifications. In some embodiments, the crRNA includes one or more locked nucleic acids for the purpose of enhancing base pairing and/or increasing nuclease resistance.

A summary of these chemical modifications can be found, e.g., in Kelley et al., “Versatility of chemically synthesized guide RNAs for CRISPR-Cas9 genome editing,” J. Biotechnol. 233:74-83, 2016; WO 2016205764; and U.S. Pat. No. 8,795,965 B2; each which is incorporated by reference in its entirety.

The sequences and the lengths of the RNA guides (e.g., crRNAs) described herein can be optimized. In some embodiments, the optimized length of an RNA guide can be determined by identifying the processed form of crRNA (i.e., a mature crRNA), or by empirical length studies for crRNA tetraloops.

The crRNAs can also include one or more aptamer sequences. Aptamers are oligonucleotide or peptide molecules have a specific three-dimensional structure and can bind to a specific target molecule. The aptamers can be specific to gene effector proteins, gene activators, or gene repressors. In some embodiments, the aptamers can be specific to a protein, which in turn is specific to and recruits and/or binds to specific gene effector proteins, gene activators, or gene repressors. The effector proteins, activators, or repressors can be present in the form of fusion proteins. In some embodiments, the guide RNA has two or more aptamer sequences that are specific to the same adaptor proteins. In some embodiments, the two or more aptamer sequences are specific to different adaptor proteins. The adaptor proteins can include, e.g., MS2, PP7, Qβ, F2, GA, fr, JP501, M12, R17, BZ13, JP34, JP500, KU1, M11, MX1, TW18, VK, SP, FI, ID2, NL95, TW19, AP205, ϕkCb5, ϕkCb8r, ϕkCb12r, ϕkCb23r, 7s, and PRR1. Accordingly, in some embodiments, the aptamer is selected from binding proteins specifically binding any one of the adaptor proteins as described herein. In some embodiments, the aptamer sequence is a MS2 binding loop. In some embodiments, the aptamer sequence is a QBeta binding loop. In some embodiments, the aptamer sequence is a PP7 binding loop. A detailed description of aptamers can be found, e.g., in Nowak et al., “Guide RNA engineering for versatile Cas9 functionality,” Nucl. Acid. Res., 44(20):9555-9564, 2016; and WO 2016205764, which are incorporated herein by reference in their entirety.

In certain embodiments, the methods make use of chemically modified guide RNAs. Examples of guide RNA chemical modifications include, without limitation, incorporation of 2′-O-methyl (M), 2′-O-methyl 3′-phosphorothioate (MS), or 2′-O-methyl 3′-thioPACE (MSP) at one or more terminal nucleotides. Such chemically modified guide RNAs can comprise increased stability and increased activity as compared to unmodified guide RNAs, though on-target vs. off-target specificity is not predictable. See, Hendel, Nat Biotechnol. 33(9):985-9, 2015, incorporated by reference). Chemically modified guide RNAs may further include, without limitation, RNAs with phosphorothioate linkages and locked nucleic acid (LNA) nucleotides comprising a methylene bridge between the 2′ and 4′ carbons of the ribose ring.

The disclosure also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein. The bacteriophage coat protein may be selected from the group comprising Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In certain embodiments, the bacteriophage coat protein is MS2.

In some embodiments, the DR sequence for the Cas13f effector protein herein is SEQ ID NO: 2.

In some embodiments, the spacer sequence is selected from SEQ ID NO: 8, 11, and 12. In some embodiments, the gRNA comprising the spacer sequence selected from SEQ ID NO: 8, 11, and 12 is used for treatment of the target sequence corresponding to the Spacer RNA sequences associated diseases. For example, the gRNA comprising the spacer sequence of SEQ ID NO: 11 is used for treatment of Rho-associated diseases, such as, PD; the gRNA comprising the spacer sequence of SEQ ID NO: 12 is used for treatment of SOD1-associated diseases, such as, ALS; the gRNA comprising the spacer sequence of SEQ ID NO: 8 is used for treatment of ATXN2-associated diseases, such as, ALS.

### 5. Target RNA

The target RNA can be any RNA molecule of interest, including naturally-occurring and engineered RNA molecules. The target RNA can be an mRNA, a tRNA, a ribosomal RNA (rRNA), a microRNA (miRNA), an interfering RNA (siRNA), a ribozyme, a riboswitch, a satellite RNA, a microswitch, a microzyme, or a viral RNA.

In some embodiments, the target nucleic acid is associated with a condition or disease (e.g., an infectious disease or a cancer).

Thus, in some embodiments, the systems described herein can be used to treat a condition or disease by targeting these nucleic acids. For instance, the target nucleic acid associated with a condition or disease may be an RNA molecule that is overexpressed in a diseased cell (e.g., a cancer or tumor cell). The target nucleic acid may also be a toxic RNA and/or a mutated RNA (e.g., an mRNA molecule having a splicing defect or a mutation). The target nucleic acid may also be an RNA that is specific for a particular microorganism (e.g., a pathogenic bacteria).

### 6. Complex and Cell

One aspect of the disclosure provides a complex of an engineered Cas13f protein, such as those either substantially lacking or having enhanced collateral activity, such as CRISPR-Cas13f complex, comprising (1) any of the engineered Cas13f protein, such as those either substantially lacking or having enhanced collateral activity (e.g., engineered Cas13f effector proteins, homologs, orthologs, fusions, derivative, conjugates, or functional fragments thereof as described herein), and (2) any of the guide RNA described herein, each including a spacer sequence designed to be at least partially complementary to a target RNA, and a DR sequence compatible with the engineered Cas13f protein, such as those either substantially lacking or having enhanced collateral activity, homologs, orthologs, fusions, derivatives, conjugates, or functional fragments thereof.

In certain embodiments, the complex further comprises the target RNA bound by the guide RNA.

In a related aspect, the disclosure also provides a cell comprising any of the complex of the disclosure.

In certain embodiments, the cell is a prokaryote. In certain embodiments, the cell is a eukaryote.

### 7. Methods of Using CRISPR Systems

The CRISPR-Cas systems having the engineered Cas13f protein, such as those either substantially lacking or having enhanced collateral activity, as described herein, have a wide variety of utilities like the corresponding wild type Cas13-based systems, including modifying (e.g., deleting, inserting, translocating, inactivating, or activating) a target polynucleotide or nucleic acid in a multiplicity of cell types. The CRISPR systems have a broad spectrum of applications in, e.g., tracking and labeling of nucleic acids, enrichment assays (extracting desired sequence from background), controlling interfering RNA or miRNA, detecting circulating tumor DNA, preparing next generation library, drug screening, disease diagnosis and prognosis, and treating various genetic disorders.

Certain engineered Cas13f effector proteins, as described herein, have enhanced collateral effect compared to the wild type, and thus may be better alternatives than the wild type Cas13f effector proteins for utilities that take advantage of the enhanced collateral activity, such as DNA/RNA detection (e.g., specific high sensitivity enzymatic reporter unlocking (SHERLOCK)). Such engineered Cas13f effector proteins with enhanced collateral activity is within the scope of one aspect of the disclosure.

### RNA Detection

In one aspect, the CRISPR systems described herein can be used in RNA detection. As shown in the examples, wild type Cas13f of the disclosure exhibit non-specific/collateral RNase activity upon activation of its guide RNA-dependent specific RNase activity when the spacer sequence is about 30 nucleotides. Thus the engineered CRISPR-associated proteins of the disclosure with enhanced collateral activity (compared to the wild type) can be reprogrammed with CRISPR RNAs (crRNAs) to provide a platform for specific RNA sensing. Further, by choosing specific spacer sequence length, and upon recognition of its RNA target, activated CRISPR-associated proteins engage in enhanced collateral cleavage of nearby non-targeted RNAs. This crRNA-programmed collateral cleavage activity allows the CRISPR systems to detect the presence of a specific RNA by triggering programmed cell death or by nonspecific degradation of labeled RNA.

The SHERLOCK method (Specific High Sensitivity Enzymatic Reporter UnLOCKing) provides an in vitro nucleic acid detection platform with attomolar sensitivity based on nucleic acid amplification and collateral cleavage of a reporter RNA, allowing for real-time detection of the target. To achieve signal detection, the detection can be combined with different isothermal amplification steps. For example, recombinase polymerase amplification (RPA) can be coupled with T7 transcription to convert amplified DNA to RNA for subsequent detection. The combination of amplification by RPA, T7 RNA polymerase transcription of amplified DNA to RNA, and detection of target RNA by collateral RNA cleavage-mediated release of reporter signal is referred as SHERLOCK. Methods of using CRISPR in SHERLOCK are described in detail, e.g., in Gootenberg, et al. “Nucleic acid detection with CRISPR-Cas13a/C2c2,” Science, 2017 Apr. 28; 356(6336):438-442, which is incorporated herein by reference in its entirety.

The disclosure described herein provides mutant/mutant Class 2, Type VI CRISPR-Cas effector proteins, especially Type VI-D, -E, and -F Cas mutants/mutants having enhanced collateral effect, such that they can be more effective in nucleic acid detection assays based on the collateral effect, such as the SHERLOCK assay. Such mutants include any one described in Examples 1 having at least 80%, 85%, or 87.5% or more collateral cleavage efficiency, and optionally better gRNA-guided cleavage compared to a corresponding wild type Cas13f.

In certain embodiments, such Cas13f mutants have enhanced collateral effect comprises, consists essentially of, or consists of a mutation corresponding to F46S15, F10S6, F10S5, F38S12, F10S4, F38S10, or F46V3 mutation in Example 1.

The CRISPR-associated proteins can be used in Northern blot assays, which use electrophoresis to separate RNA samples by size. The CRISPR-associated proteins can be used to specifically bind and detect the target RNA sequence. The CRISPR-associated proteins can also be fused to a fluorescent protein (e.g., GFP) and used to track RNA localization in living cells. More particularly, the CRISPR-associated proteins can be inactivated in that they no longer cleave RNAs as described above. Thus, CRISPR-associated proteins can be used to determine the localization of the RNA or specific splice mutants, the level of mRNA transcripts, up- or down-regulation of transcripts and disease-specific diagnosis. The CRISPR-associated proteins can be used for visualization of RNA in (living) cells using, for example, fluorescent microscopy or flow cytometry, such as fluorescence-activated cell sorting (FACS), which allows for high-throughput screening of cells and recovery of living cells following cell sorting. A detailed description regarding how to detect DNA and RNA can be found, e.g., in International Publication No. WO 2017/070605, which is incorporated herein by reference in its entirety. In some embodiments, the CRISPR systems described herein can be used in multiplexed error-robust fluorescence in situ hybridization (MERFISH). These methods are described in, e.g., Chen et al., “Spatially resolved, highly multiplexed RNA profiling in single cells,” Science, 2015 Apr. 24; 348(6233):aaa6090, which is incorporated herein by reference herein in its entirety.

In some embodiments, the CRISPR systems described herein can be used to detect a target RNA in a sample (e.g., a clinical sample, a cell, or a cell lysate). The collateral RNase activity of the engineered Cas13f, e.g., Cas13f effector proteins described herein, is activated when the effector proteins bind to a target nucleic acid when the spacer sequence is of a specific chosen length (such as about 30 nucleotides). Upon binding to the target RNA of interest, the effector protein cleaves a labeled detector RNA to generate a signal (e.g., an increased signal or a decreased signal) thereby allowing for the qualitative and quantitative detection of the target RNA in the sample. The specific detection and quantification of RNA in the sample allows for a multitude of applications including diagnostics. In some embodiments, the methods include contacting a sample with: i) an RNA guide (e.g., crRNA) and/or a nucleic acid encoding the RNA guide, wherein the RNA guide consists of a direct repeat sequence and a spacer sequence capable of hybridizing to the target RNA; (ii) an engineered Cas13f protein with enhanced collateral activity compared to wild type Cas13f, such as a subject engineered Cas13f effector protein and/or a nucleic acid encoding the effector protein; and (iii) a labeled detector RNA; wherein the effector protein associates with the RNA guide to form a complex; wherein the RNA guide hybridizes to the target RNA; and wherein upon binding of the complex to the target RNA, the effector protein exhibits collateral RNase activity and cleaves the labeled detector RNA; and b) measuring a detectable signal produced by cleavage of the labeled detector RNA, wherein the measuring provides for detection of the single-stranded target RNA in the sample. In some embodiments, the methods further comprise comparing the detectable signal with a reference signal and determining the amount of target RNA in the sample.

In some embodiments, the measuring is performed using gold nanoparticle detection, fluorescence polarization, colloid phase transition/dispersion, electrochemical detection, and semiconductor based-sensing. In some embodiments, the labeled detector RNA includes a fluorescence-emitting dye pair, a fluorescence resonance energy transfer (FRET) pair, or a quencher/fluor pair. In some embodiments, upon cleavage of the labeled detector RNA by the effector protein, an amount of detectable signal produced by the labeled detector RNA is decreased or increased. In some embodiments, the labeled detector RNA produces a first detectable signal prior to cleavage by the effector protein and a second detectable signal after cleavage by the effector protein. In some embodiments, a detectable signal is produced when the labeled detector RNA is cleaved by the effector protein. In some embodiments, the labeled detector RNA comprises a modified nucleobase, a modified sugar moiety, a modified nucleic acid linkage, or a combination thereof. In some embodiments, the methods include the multi-channel detection of multiple independent target RNAs in a sample (e.g., two, three, four, five, six, seven, eight, nine, ten, fifteen, twenty, thirty, forty, or more target RNAs) by using multiple engineered Cas13f, such as the engineered CRISPR-Cas13f systems of the disclosure, each including a distinct orthologous effector protein and corresponding RNA guides, allowing for the differentiation of multiple target RNAs in the sample. In some embodiments, the methods include the multi-channel detection of multiple independent target RNAs in a sample, with the use of multiple instances of engineered Cas13f, such as engineered CRISPR-Cas13f systems of the disclosure, each containing an orthologous effector protein with differentiable collateral RNase substrates. Methods of detecting an RNA in a sample using CRISPR-associated proteins are described, for example, in U.S. Patent Publication No. 2017/0362644, the entire contents of which are incorporated herein by reference.

### Tracking and Labeling of Nucleic Acids

Cellular processes depend on a network of molecular interactions among proteins, RNAs, and DNAs. Accurate detection of protein-DNA and protein-RNA interactions is key to understanding such processes. In vitro proximity labeling techniques employ an affinity tag combined with, a reporter group, e.g., a photoactivatable group, to label polypeptides and RNAs in the vicinity of a protein or RNA of interest in vitro. After UV irradiation, the photoactivatable groups react with proteins and other molecules that are in close proximity to the tagged molecules, thereby labelling them. Labelled interacting molecules can subsequently be recovered and identified. The CRISPR-associated proteins can for instance be used to target probes to selected RNA sequences. These applications can also be applied in animal models for in vivo imaging of diseases or difficult-to culture cell types. The methods of tracking and labeling of nucleic acids are described, e.g., in U.S. Pat. No. 8,795,965, WO 2016205764, and WO 2017070605; each of which is incorporated herein by reference herein in its entirety.

RNA Isolation, Purification, Enrichment, and/or Depletion

The CRISPR systems (e.g., CRISPR-associated proteins) described herein can be used to isolate and/or purify the RNA. The CRISPR-associated proteins can be fused to an affinity tag that can be used to isolate and/or purify the RNA-CRISPR-associated protein complex. These applications are useful, e.g., for the analysis of gene expression profiles in cells.

In some embodiments, the CRISPR-associated proteins can be used to target a specific noncoding RNA (ncRNA) thereby blocking its activity. In some embodiments, the CRISPR-associated proteins can be used to specifically enrich a particular RNA (including but not limited to increasing stability, etc.), or alternatively, to specifically deplete a particular RNA (e.g., particular splice mutants, isoforms, etc.).

These methods are described, e.g., in U.S. Pat. No. 8,795,965, WO 2016205764, and WO 2017070605; each of which is incorporated herein by reference herein in its entirety.

### High-Throughput Screening

The CRISPR systems described herein can be used for preparing next generation sequencing (NGS) libraries. For example, to create a cost-effective NGS library, the CRISPR systems can be used to disrupt the coding sequence of a target gene product, and the CRISPR-associated protein transfected clones can be screened simultaneously by next-generation sequencing (e.g., on the Ion Torrent PGM system). A detailed description regarding how to prepare NGS libraries can be found, e.g., in Bell et al., “A high-throughput screening strategy for detecting CRISPR-Cas9 induced mutations using next-generation sequencing,” BMC Genomics, 15.1 (2014): 1002, which is incorporated herein by reference in its entirety.

### Engineered Microorganisms

Microorganisms (e.g., E. coli, yeast, and microalgae) are widely used for synthetic biology. The development of synthetic biology has a wide utility, including various clinical applications. For example, the programmable CRISPR systems can be used to split proteins of toxic domains for targeted cell death, e.g., using cancer-linked RNA as target transcript. Further, pathways involving protein-protein interactions can be influenced in synthetic biological systems with, e.g., fusion complexes with the appropriate effector proteins such as kinases or enzymes.

In some embodiments, crRNAs that target phage sequences can be introduced into the microorganism. Thus, the disclosure also provides methods of vaccinating a microorganism (e.g., a production strain) against phage infection.

In some embodiments, the CRISPR systems provided herein can be used to engineer microorganisms, e.g., to improve yield or improve fermentation efficiency. For example, the CRISPR systems described herein can be used to engineer microorganisms, such as yeast, to generate biofuel or biopolymers from fermentable sugars, or to degrade plant-derived lignocellulose derived from agricultural waste as a source of fermentable sugars. More particularly, the methods described herein can be used to modify the expression of endogenous genes required for biofuel production and/or to modify endogenous genes, which may interfere with the biofuel synthesis. These methods of engineering microorganisms are described e.g., in Verwaal et al., “CRISPR/Cpf1 enables fast and simple genome editing of Saccharomyces cerevisiae,” Yeast doi: 10.1002/yea.3278, 2017; and Hlavova et al., “Improving microalgae for biotechnology—from genetics to synthetic biology,” Biotechnol. Adv., 33:1194-203, 2015, both of which are incorporated herein by reference in the entirety.

In some embodiments, the CRISPR systems provided herein can be used to induce death or dormancy of a cell (e.g., a microorganism such as an engineered microorganism). These methods can be used to induce dormancy or death of a multitude of cell types including prokaryotic and eukaryotic cells, including, but not limited to mammalian cells (e.g., cancer cells, or tissue culture cells), protozoans, fungal cells, cells infected with a virus, cells infected with an intracellular bacteria, cells infected with an intracellular protozoan, cells infected with a prion, bacteria (e.g., pathogenic and non-pathogenic bacteria), protozoans, and unicellular and multicellular parasites. For instance, in the field of synthetic biology it is highly desirable to have mechanisms of controlling engineered microorganisms (e.g., bacteria) in order to prevent their propagation or dissemination. The systems described herein can be used as “kill-switches” to regulate and/or prevent the propagation or dissemination of an engineered microorganism. Further, there is a need in the art for alternatives to current antibiotic treatments. The systems described herein can also be used in applications where it is desirable to kill or control a specific microbial population (e.g., a bacterial population). For example, the systems described herein may include an RNA guide (e.g., a crRNA) that targets a nucleic acid (e.g., an RNA) that is genus-, species-, or strain-specific, and can be delivered to the cell. Upon complexing and binding to the target nucleic acid, the collateral RNase activity of the Cas13f effector proteins is activated leading to the cleavage of non-target RNA within the microorganisms, ultimately resulting in dormancy or death. In some embodiments, the methods comprise contacting the cell with a system described herein including a Cas13f effector proteins or a nucleic acid encoding the effector protein, and a RNA guide (e.g., a crRNA) or a nucleic acid encoding the RNA guide, wherein the spacer sequence is complementary to at least 15 nucleotides (e.g., 16, 17, 18, 19, 20, 21, 22, 23, 24, 25, 26, 27, 28, 29, 30, 35, 40, 45, 50 or more nucleotides) of a target nucleic acid (e.g., a genus-, strain-, or species-specific RNA guide). Without wishing to be bound by any particular theory, the cleavage of non-target RNA by the Cas13f effector proteins may induce programmed cell death, cell toxicity, apoptosis, necrosis, necroptosis, cell death, cell cycle arrest, cell anergy, a reduction of cell growth, or a reduction in cell proliferation. For example, in bacteria, the cleavage of non-target RNA by the Cas13f effector proteins may be bacteriostatic or bactericidal.

### Application in Plants

The CRISPR systems described herein have a wide variety of utility in plants. In some embodiments, the CRISPR systems can be used to engineer transcriptome of plants (e.g., improving production, making products with desired post-translational modifications, or introducing genes for producing industrial products). In some embodiments, the CRISPR systems can be used to introduce a desired trait to a plant (e.g., without heritable modifications to the genome), or regulate expression of endogenous genes in plant cells or whole plants.

In some embodiments, the CRISPR systems can be used to identify, edit, and/or silence genes encoding specific proteins, e.g., allergenic proteins (e.g., allergenic proteins in peanuts, soybeans, lentils, peas, green beans, and mung beans). A detailed description regarding how to identify, edit, and/or silence genes encoding proteins is described, e.g., in Nicolaou et al., “Molecular diagnosis of peanut and legume allergy,” Curr Opin. Allergy Clin. Immunol. 11(3):222-8, 2011, and WO 2016205764 A1; both of which are incorporated herein by reference in the entirety.

### Pooled-Screening

As described herein, pooled CRISPR screening is a powerful tool for identifying genes involved in biological mechanisms such as cell proliferation, drug resistance, and viral infection. Cells are transduced in bulk with a library of guide RNA (gRNA)-encoding vectors described herein, and the distribution of gRNAs is measured before and after applying a selective challenge. Pooled CRISPR screens work well for mechanisms that affect cell survival and proliferation, and they can be extended to measure the activity of individual genes (e.g., by using engineered reporter cell lines). Arrayed CRISPR screens, in which only one gene is targeted at a time, make it possible to use RNA-seq as the readout. In some embodiments, the CRISPR systems as described herein can be used in single-cell CRISPR screens. A detailed description regarding pooled CRISPR screenings can be found, e.g., in Datlinger et al., “Pooled CRISPR screening with single-cell transcriptome read-out,” Nat. Methods. 14(3):297-301, 2017, which is incorporated herein by reference in its entirety.

### Saturation Mutagenesis (Bashing)

The CRISPR systems described herein can be used for in situ saturating mutagenesis. In some embodiments, a pooled guide RNA library can be used to perform in situ saturating mutagenesis for particular genes or regulatory elements. Such methods can reveal critical minimal features and discrete vulnerabilities of these genes or regulatory elements (e.g., enhancers). These methods are described, e.g., in Canver et al., “BCL11A enhancer dissection by Cas9-mediated in situ saturating mutagenesis,” Nature 527(7577):192-7, 2015, which is incorporated herein by reference in its entirety.

### RNA-Related Applications

The CRISPR systems described herein can have various RNA-related applications, e.g., modulating gene expression, degrading a RNA molecule, inhibiting RNA expression, screening RNA or RNA products, determining functions of lincRNA or non-coding RNA, inducing cell dormancy, inducing cell cycle arrest, reducing cell growth and/or cell proliferation, inducing cell anergy, inducing cell apoptosis, inducing cell necrosis, inducing cell death, and/or inducing programmed cell death. A detailed description of these applications can be found, e.g., in WO 2016/205764 A1, which is incorporated herein by reference in its entirety. In different embodiments, the methods described herein can be performed in vitro, in vivo, or ex vivo.

For example, the CRISPR systems described herein can be administered to a subject having a disease or disorder to target and induce cell death in a cell in a diseased state (e.g., cancer cells or cells infected with an infectious agent). For instance, in some embodiments, the CRISPR systems described herein can be used to target and induce cell death in a cancer cell, wherein the cancer cell is from a subject having a Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, or urinary bladder cancer.

### Modulating Gene Expression

The CRISPR systems described herein can be used to modulate gene expression. The CRISPR systems can be used, together with suitable guide RNAs, to target gene expression, via control of RNA processing. The control of RNA processing can include, e.g., RNA processing reactions such as RNA splicing (e.g., alternative splicing), viral replication, and tRNA biosynthesis. The RNA targeting proteins in combination with suitable guide RNAs can also be used to control RNA activation (RNAa). RNA activation is a small RNA-guided and Argonaute (Ago)-dependent gene regulation phenomenon in which promoter-targeted short double-stranded RNAs (dsRNAs) induce target gene expression at the transcriptional/epigenetic level. RNAa leads to the promotion of gene expression, so control of gene expression may be achieved that way through disruption or reduction of RNAa. In some embodiments, the methods include the use of the RNA targeting CRISPRas substitutes for e.g., interfering ribonucleic acids (such as siRNAs, shRNAs, or dsRNAs). The methods of modulating gene expression are described, e.g., in WO 2016205764, which is incorporated herein by reference in its entirety.

### Controlling RNA Interference

Control over interfering RNAs or microRNAs (miRNA) can help reduce off-target effects by reducing the longevity of the interfering RNAs or miRNAs in vivo or in vitro. In some embodiments, the target RNAs can include interfering RNAs, i.e., RNAs involved in the RNA interference pathway, such as small hairpin RNAs (shRNAs), small interfering (siRNAs), etc. In some embodiments, the target RNAs include, e.g., miRNAs or double stranded RNAs (dsRNA).

In some embodiments, if the RNA targeting protein and suitable guide RNAs are selectively expressed (for example spatially or temporally under the control of a regulated promoter, for example a tissue- or cell cycle-specific promoter and/or enhancer), this can be used to protect the cells or systems (in vivo or in vitro) from RNA interference (RNAi) in those cells. This may be useful in neighboring tissues or cells where RNAi is not required or for the purposes of comparison of the cells or tissues where the CRISPR-associated proteins and suitable crRNAs are and are not expressed (i.e., where the RNAi is not controlled and where it is, respectively). The RNA targeting proteins can be used to control or bind to molecules comprising or consisting of RNAs, such as ribozymes, ribosomes, or riboswitches. In some embodiments, the guide RNAs can recruit the RNA targeting proteins to these molecules so that the RNA targeting proteins are able to bind to them. These methods are described, e.g., in WO 2016205764 and WO 2017070605, both of which are incorporated herein by reference in the entirety.

### Modifying Riboswitches and Controlling Metabolic Regulations

Riboswitches are regulatory segments of messenger RNAs that bind small molecules and in turn regulate gene expression. This mechanism allows the cell to sense the intracellular concentration of these small molecules. A specific riboswitch typically regulates its adjacent gene by altering the transcription, the translation or the splicing of this gene. Thus, in some embodiments, the riboswitch activity can be controlled by the use of the RNA targeting proteins in combination with suitable guide RNAs to target the riboswitches. This may be achieved through cleavage of, or binding to, the riboswitch. Methods of using CRISPR systems to control riboswitches are described, e.g., in WO 2016205764 and WO 2017070605, both of which are incorporated herein by reference in their entireties.

### RNA Modification

In some embodiments, the CRISPR-associated proteins described herein can be fused to a base-editing domain, such as ADAR1, ADAR2, APOBEC, or activation-induced cytidine deaminase (AID), and can be used to modify an RNA sequence (e.g., an mRNA). In some embodiments, the CRISPR-associated protein includes one or more mutations (e.g., in a catalytic domain), which renders the subject CRISPR-associated protein incapable of cleaving RNA (e.g., the dCas13f version of the engineered Cas13f protein described herein).

In some embodiments, such CRISPR-associated proteins can be used with an RNA-binding fusion polypeptide comprising a base-editing domain (e.g., ADARI, ADAR2, APOBEC, or AID) fused to an RNA-binding domain, such as MS2 (also known as MS2 coat protein), Qbeta (also known as Qbeta coat protein), or PP7 (also known as PP7 coat protein).

In some embodiments, the RNA binding domain can bind to a specific sequence (e.g., an aptamer sequence) or secondary structure motifs on a crRNA of the system described herein (e.g., when the crRNA is in an effector-crRNA complex), thereby recruiting the RNA binding fusion polypeptide (which has a base-editing domain) to the effector complex. For example, in some embodiments, the CRISPR system includes a CRISPR associated protein, a crRNA having an aptamer sequence (e.g., an MS2 binding loop, a QBeta binding loop, or a PP7 binding loop), and a RNA-binding fusion polypeptide having a base-editing domain fused to an RNA-binding domain that specifically binds to the aptamer sequence. In this system, the CRISPR-associated protein forms a complex with the crRNA having the aptamer sequence. Further the RNA-binding fusion polypeptide binds to the crRNA (via the aptamer sequence) thereby forming a tripartite complex that can modify a target RNA.

Methods of using CRISPR systems for base editing are described, e.g., in International Publication No. WO 2017/219027, which is incorporated herein by reference in its entirety, and in particular with respect to its discussion of RNA modification.

### RNA Splicing

In some embodiments, an inactivated or dCas13f version of the engineered Cas13f protein substantially lacking collateral activity described herein (e.g., an engineered CRISPR associated protein having one or more further mutations in a catalytic domain) can be used to target and bind to specific splicing sites on RNA transcripts. Binding of the inactivated CRISPR-associated protein to the RNA may sterically inhibit interaction of the spliceosome with the transcript, enabling alteration in the frequency of generation of specific transcript isoforms. Such method can be used to treat disease through exon skipping such that an exon having a mutation may be skipped in a mature protein. Methods of using CRISPR systems to alter splicing are described, e.g., in International Publication No. WO 2017/219027, which is incorporated herein by reference in its entirety, and in particular with respect to its discussion of RNA splicing.

### Therapeutic Applications

The CRISPR systems described herein can have various therapeutic applications. Such applications may be based on one or more of the abilities below, both in vitro and in vivo, of the subject engineered Cas13f, e.g., engineered CRISPR-Cas3f systems: induce cellular senescence, induce cell cycle arrest, inhibit cell growth and/or proliferation, induce apoptosis, induce necrosis, etc.

In some embodiments, the new engineered CRISPR systems can be used to treat various diseases and disorders, e.g., genetic disorders (e.g., monogenetic diseases), diseases that can be treated by nuclease activity (e.g., Pcsk9 targeting, Duchenne Muscular Dystrophy (DMD), BCL11a targeting), and various cancers, etc.

In some embodiments, the CRISPR systems described herein can be used to edit a target nucleic acid to modify the target nucleic acid (e.g., by inserting, deleting, or mutating one or more nucleic acid residues).

In one aspect, the CRISPR systems described herein can be used for treating a disease caused by overexpression of RNAs, toxic RNAs, and/or mutated RNAs (e.g., splicing defects or truncations). For example, expression of toxic RNAs may be associated with the formation of nuclear inclusions and late-onset degenerative changes in brain, heart, or skeletal muscle. In some embodiments, the disorder is myotonic dystrophy. In myotonic dystrophy, the main pathogenic effect of the toxic RNAs is to sequester binding proteins and compromise the regulation of alternative splicing (see, e.g., Osborne et al., “RNA-dominant diseases,” Hum. Mol. Genet., 2009 Apr. 15; 18(8):1471-81). Myotonic dystrophy (dystrophia myotonica (DM)) is of particular interest to geneticists because it produces an extremely wide range of clinical features. The classical form of DM, which is now called DM type 1 (DM1), is caused by an expansion of CTG repeats in the 3′-untranslated region (UTR) of DMPK, a gene encoding a cytosolic protein kinase. The CRISPR systems as described herein can target overexpressed RNA or toxic RNA, e.g., the DMPK gene or any of the mis-regulated alternative splicing in DM1 skeletal muscle, heart, or brain.

The CRISPR systems described herein can also target trans-acting mutations affecting RNA-dependent functions that cause various diseases such as, e.g., Prader Willi syndrome, Spinal muscular atrophy (SMA), and Dyskeratosis congenita. A list of diseases that can be treated using the CRISPR systems described herein is summarized in Cooper et al., “RNA and disease,” Cell, 136.4 (2009): 777-793, and WO 2016/205764 A1, both of which are incorporated herein by reference in the entirety. Those of skill in this field will understand how to use the new CRISPR systems to treat these diseases.

The CRISPR systems described herein can also be used in the treatment of various tauopathies, including, e.g., primary and secondary tauopathies, such as primary age-related tauopathy (PART)/Neurofibrillary tangle (NFT)-predominant senile dementia (with NFTs similar to those seen in Alzheimer Disease (AD), but without plaques), dementia pugilistica (chronic traumatic encephalopathy), and progressive supranuclear palsy. A useful list of tauopathies and methods of treating these diseases are described, e.g., in WO 2016205764, which is incorporated herein by reference in its entirety.

The CRISPR systems described herein can also be used to target mutations disrupting the cis-acting splicing codes that can cause splicing defects and diseases. These diseases include, e.g., motor neuron degenerative disease that results from deletion of the SMN1 gene (e.g., spinal muscular atrophy), Duchenne Muscular Dystrophy (DMD), frontotemporal dementia, and Parkinsonism linked to chromosome 17 (FTDP-17), and cystic fibrosis.

The CRISPR systems described herein can further be used for antiviral activity, in particular against RNA viruses. The CRISPR-associated proteins can target the viral RNAs using suitable guide RNAs selected to target viral RNA sequences.

The CRISPR systems described herein can also be used to treat a cancer in a subject (e.g., a human subject). For example, the CRISPR-associated proteins described herein can be programmed with crRNA targeting a RNA molecule that is aberrant (e.g., comprises a point mutation or are alternatively-spliced) and found in cancer cells to induce cell death in the cancer cells (e.g., via apoptosis).

The CRISPR systems described herein can also be used to treat an autoimmune disease or disorder in a subject (e.g., a human subject). For example, the CRISPR-associated proteins described herein can be programmed with crRNA targeting a RNA molecule that is aberrant (e.g., comprises a point mutation or are alternatively-spliced) and found in cells responsible for causing the autoimmune disease or disorder.

Further, the CRISPR systems described herein can also be used to treat an infectious disease in a subject. For example, the CRISPR-associated proteins described herein can be programmed with crRNA targeting a RNA molecule expressed by an infectious agent (e.g., a bacteria, a virus, a parasite or a protozoan) in order to target and induce cell death in the infectious agent cell. The CRISPR systems may also be used to treat diseases where an intracellular infectious agent infects the cells of a host subject. By programming the CRISPR-associated protein to target a RNA molecule encoded by an infectious agent gene, cells infected with the infectious agent can be targeted and cell death induced.

Furthermore, in vitro RNA sensing assays can be used to detect specific RNA substrates. The CRISPR-associated proteins can be used for RNA-based sensing in living cells. Examples of applications are diagnostics by sensing of, for examples, disease-specific RNAs.

A detailed description of therapeutic applications of the CRISPR systems described herein can be found, e.g., in U.S. Pat. No. 8,795,965, EP 3009511, WO 2016205764, and WO 2017070605; each of which is incorporated herein by reference in its entirety.

In some embodiments, the target RNA is a transcript (e.g., mRNA) of a target gene associated with an eye disease or disorder.

In some embodiments, the eye disease or disorder is amoebic keratitis, fungal keratitis, bacterial keratitis, viral keratitis, onchorcercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA), Usher's syndrome, choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof.

In some embodiments, the target gene is selected from the group consisting of Vascular Endothelial Growth Factor A (VEGFA), complement factor H (CFH), age-related maculopathy susceptibility 2 (ARMS2), HtrA serine peptidase 1 (HTRA1), ATP Binding Cassette Subfamily A Member 4 (ABCA4), Peripherin-2 (PRPH2), fibulin-5 (FBLN5), ERCC Excision Repair 6 Chromatin Remodeling Factor (ERCC6), Retina And Anterior Neural Fold Homeobox 2 (RAX2), Complement C3 (C3), Toll Like Receptor 4 (TLR4), Cystatin C (CST3), CX3C Chemokine Receptor 1 (CX3CR1), complement factor I (CFI), Complement C2 (C2), Complement Factor B (CFB), Complement C9 (C9), Mitochondrially Encoded TRNA Leucine 1 (UUA/G) (MT-TL-1), Complement Factor H Related 1 (CFHR1), Complement Factor H Related 3 (CFHR3), Ciliary Neurotrophic Factor (CNTF), pigment epithelium-derived factor (PEDF), rod-derived cone viability factor (RdCVF), glial-derived neurotrophic factor (GDNF), Myosin VIIA (MYO7A); Centrosomal Protein 290 (CEP290), Cadherin Related 23 (CDH23), Eyes Shut Homolog (EYS), Usherin (USH2A), adhesion G protein-coupled receptor VI (ADGRV1), ALMS1 Centrosome And Basal Body Associated Protein (ALMS1), Retinoid Isomerohydrolase 65 kDa (RPE65), Aryl-hydrocarbon-interacting protein-like 1 (AIPL1), Guanylate Cyclase 2D, Retinal (GUCY2D), Leber Congenital Amaurosis 5 Protein (LCA5), Cone-Rod Homeobox (CRX), Clarin (CLRN1), ATP Binding Cassette Subfamily A Member 4 (ABCA4), Retinol Dehydrogenase 12 (RDH12), Inosine Monophosphate Dehydrogenase 1 (IMPDH1), Crumbs Cell Polarity Complex Component 1 (CRB1), Lecithin retinol acyltransferase (LRAT), Nicotinamide Nucleotide Adenylyltransferase 1 (NMNAT1), TUB Like Protein 1 (TULP1), MER Proto-Oncogene, Tyrosine Kinase (MERTK), Retinitis Pigmentosa GTPase Regulato (RPGR), RP2 Activator Of ARL3 GTPase (RP2), X-linked retinitis pigmentosa GTPase regulator-interacting protein 1 (RPGRIP), Cyclic Nucleotide Gated Channel Subunit Alpha 3 (CNGA3), Cyclic Nucleotide Gated Channel Subunit Beta 3 (CNGB3), G Protein Subunit Alpha Transducin 2 (GNAT2), Fibroblast Growth Factor 2 (FGF2), Erythropoietin (EPO), BCL2 Apoptosis Regulator (BCL2), BCL2 Like 1 (BCL2L1), Nuclear Factor Kappa B (NFκB), Endostatin, Angiostatin, fis-like tyrosine kinase receptor (sFlt), Pigment-dispersing factor receptor (Pdfr), Interleukin 10 (IL10), soluble interleukin 17 (sIL17R), Interleukin-1-receptor antagonist (IL1-ra), TNF Receptor Superfamily Member 1A (TNFRSF1A), TNF Receptor Superfamily Member 1B (TNFRSF1B), and interleukin 4 (IL4).

In some embodiments, the target RNA is a transcript (e.g., mRNA) of a target gene associated with a neurodegenerative disease or disorder.

In some embodiments, the neurodegenerative disease or disorder is alcoholism, Alexander's disease, Alper's disease, Alzheimer's Disease, amyotrophic lateral sclerosis (ALS), ataxia telangiectasia, neuronal ceroid lipofuscinoses, Batten disease, bovine spongiform encephalopathy (BSE), Canavan disease, cerebral palsy, Cockayne syndrome, corticobasal degeneration, Creutzfeldt-Jakob disease, frontotemporal lobar degeneration, Huntington's disease, HIV-associated dementia, Kennedy's disease, Lewy body dementia, neuroborreliosis, primary age-related tauopathy (PART)/Neurofibrillary tangle-predominant senile dementia, Machado-Joseph disease, multiple system atrophy, multiple sclerosis, multiple sulfatase deficiency, mucolipidoses, narcolepsy, Niemann Pick disease, Parkinson's Disease, Pick's disease, Pompe disease, primary lateral sclerosis, prion diseases, neuronal loss, cognitive defect, motor neuron diseases, Duchenne Muscular Dystrophy (DMD), frontotemporal dementia, frontotemporal dementia and parkinsonism linked to chromosome 17, Lytico-Bodig disease (Parkinson-dementia complex of Guam), neuroaxonal dystrophies, Refsum's disease, Schilder's disease, subacute combined degeneration of spinal cord secondary to pernicious anaemia, Spielmeyer-Vogt-Sjogren-Batten disease, Parkinsonism linked to chromosome 17 (FTDP-17), Prader Willi syndrome, Myotonic dystrophy, chronic traumatic encephalopathy including dementia pugilistica, spinocerebellar ataxia, spinal muscular atrophy, Steele-Richardson-Olszewski disease, Tabes dorsalis, Niemann-Pick Type C (NPC1 and/or NPC2 defect), Smith-Lemli-Opitz Syndrome (SLOS), an inborn error of cholesterol synthesis, Tangier disease, Pelizaeus-Merzbacher disease, a neuronal ceroid lipofuscinosis, a primary glycosphingolipidosis, Farber disease or multiple sulphatase deficiency, Gaucher disease, Fabry disease, GM1 gangliosidosis, GM2 gangliosidosis, Krabbe disease, metachromatic leukodystrophy (MLD), NPC, GM1 gangliosidosis, Fabry disease, a neurodegenerative mucopolysaccharidosis, MPS I, MPS IH, MPS IS, MPS II, MPS III, MPS IIIA, MPS IIIB, MPS IIIC, MPS HID, MPS, IV, MPS IV A, MPS IV B, MPS VI, MPS VII, MPS IX, a disease with secondary lysosomal involvement, SLOS, Tangier disease, ganglioglioma, gangliocytoma, meningioangiomatosis, postencephalitic parkinsonism, subacute sclerosing panencephalitis, lead encephalopathy, tuberous sclerosis, Hallervorden-Spatz disease, lipofuscinosis, cerebellar ataxia, parkinsonism, Louis-Barr syndrome, multiple systems atrophy, fronto-temporal dementia or lower body Parkinson's syndrome, Niemann Pick disease, Niemann Pick type C, Niemann Pick type A, Tay-Sachs disease, multisystemic atrophy cerebellar type (MSA-C), fronto-temporal dementia with parkinsonism, progressive supranuclear palsy, cerebellar downbeat nystagmus, Sandhoff's disease or mucolipidosis type II, or combinations thereof.

In some embodiments, the target RNA is a transcript (e.g., mRNA) of a target gene associated with a cancer.

In some embodiments, the cancer is carcinomas, sarcomas, myelomas, leukemias, lymphomas and mixed type tumors. Non-limiting examples of cancers that may treated by methods and compositions described herein include, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; plasmacytoma, colorectal cancer, rectal cancer, and hairy cell leukemia.

In some embodiments, the target RNA is a transcript (e.g., mRNA) associated with a disease selected from the group consisting of: (shown in the format of “disease or disorder—causal gene or transcript”)

### Neuronal:

Rett syndrome—MECP2,
MDS—MECP2,
Angles syndrome—UBE3A-ATS,
AADC deficiency—AADC,
Canavan disease—ASPA,
Late infantile neuronal ceroid lipofuscinosis—CLN2 (also known as TPP1),
Friedreich ataxia—FRDA (also known as FXN),
Giant axonal neuropathy—GAN,
Leber's Hereditary Optic Neuropathy—ND1/ND4;

### Ocular:

Achromatopsia—CNGA3,
Leber Congenital Amaurosis 10 Protein—CEP290,
Retinitis Pigmentosa—RHO;

### Muscular:

Dysferlinopathy—DYSF,

### Danon Disease—LAMP2,

Myotonic dystrophy type 1 (DM1)—DMPK;

### Auditory:

Pendred syndrome—SLC26A4,
Wolfram syndrome—WFS1,
Stickler syndrome—COL11A2,
Nonsyndromic hearing loss—GJB2/OTOF/Myo6/STRC/KCNQ4/TECTA;

### Hepatic:

Homozygous Familial Hypercholesterolemia—LDLR/PCSK9,
Alpha—1 antitrypsin deficiency—SERPINA1;

### Others:

Phenylketonuria—phenylalanine hydroxylase (PAH),
Crigler—Najjar Syndrome—UGT1A1,
Ornithine transcarbamylase (OTC) deficiency—OTC,
Glycogen Storage Disease Type IA—G6Pase.

### Cells and Progenies Thereof

In certain embodiments, the methods of the disclosure can be used to introduce the CRISPR systems described herein into a cell, and cause the cell and/or its progeny to alter the production of one or more cellular produces, such as antibody, starch, ethanol, or any other desired products. Such cells and progenies thereof are within the scope of the disclosure.

In certain embodiments, the methods and/or the CRISPR systems described herein lead to modification of the translation and/or transcription of one or more RNA products of the cells. For example, the modification may lead to increased transcription/translation/expression of the RNA product. In other embodiments, the modification may lead to decreased transcription/translation/expression of the RNA product.

In certain embodiments, the cell is a prokaryotic cell.

In certain embodiments, the cell is a eukaryotic cell, such as a mammalian cell, including a human cell (a primary human cell or an established human cell line). In certain embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In certain embodiments, the cell is from fish (such as salmon), bird (such as poultry bird, including chick, duck, goose), reptile, shellfish (e.g., oyster, claim, lobster, shrimp), insect, worm, yeast, etc. In certain embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat.

In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

A related aspect provides cells or progenies thereof modified by the methods of the disclosure using the CRISPR systems described herein.

In certain embodiments, the cell is modified in vitro, in vivo, or ex vivo.

In certain embodiments, the cell is a stem cell.

### 8. Delivery

Through this disclosure and the knowledge in the art, the CRISPR systems described herein comprising an engineered Cas13f protein, such as those either substantially lacking or having enhanced collateral activity, or any of the components thereof described herein (Cas13f proteins, derivatives, functional fragments, or the various fusions or adducts thereof, and guide RNA/crRNA), nucleic acid molecules thereof, and/or nucleic acid molecules encoding or providing components thereof, can be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In certain embodiments, the CRISPR-associated proteins and/or any of the RNAs (e.g., guide RNAs or crRNAs) and/or accessory proteins can be delivered using suitable vectors, e.g., plasmids or viral vectors, such as adeno-associated viruses (AAV), lentiviruses, adenoviruses, retroviral vectors, and other viral vectors, or combinations thereof. The proteins and one or more crRNAs can be packaged into one or more vectors, e.g., plasmids or viral vectors. For bacterial applications, the nucleic acids encoding any of the components of the CRISPR systems described herein can be delivered to the bacteria using a phage. Exemplary phages, include, but are not limited to, T4 phage, Mu, λ, phage, T5 phage, T7 phage, T3 phage, 029, M13, MS2, Qβ, and ΦX174. Instead of packaging a single strand (ss)DNA sequence as a vector genome of a AAV particle, systems and methods of packaging an RNA sequence as a vector genome into a AAV particle is recently developed and applicable herein. See PCT/CN2022/075366, which is incorporated herein by reference in its entirety.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×10⁵ particles (also referred to as particle units, pu) of adenoviruses. In some embodiments, the dose preferably is at least about 1×10⁶ particles, at least about 1×10′ particles, at least about 1×10⁸ particles, and at least about 1×10⁶ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a nucleic acid-targeting CRISPR-associated proteins and/or an accessory protein, each operably linked to a promoter (e.g., the same promoter or a different promoter); (iii) a selectable marker; (iv) an origin of replication; and (v) a transcription terminator downstream of and operably linked to (ii). The plasmids can also encode the RNA components of a CRISPR complex, but one or more of these may instead be encoded on different vectors. The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

Further means of introducing one or more components of the new CRISPR systems to the cell is by using cell penetrating peptides (CPP). In some embodiments, a cell penetrating peptide is linked to the CRISPR-associated proteins. In some embodiments, the CRISPR-associated proteins and/or guide RNAs are coupled to one or more CPPs to effectively transport them inside cells (e.g., plantprotoplasts).

In some embodiments, the CRISPR-associated proteins and/or guide RNA(s) are encoded by one or more circular or non-circular DNA molecules that are coupled to one or more CPPs for cell delivery. CPPs are short peptides of fewer than 35 amino acids derived either from proteins or from chimeric sequences capable of transporting biomolecules across cell membrane in a receptor independent manner.

CPPs can be cationic peptides, peptides having hydrophobic sequences, amphipathic peptides, peptides having proline-rich and anti-microbial sequences, and chimeric or bipartite peptides. Examples of CPPs include, e.g., Tat (which is a nuclear transcriptional activator protein required for viral replication by HIV type 1), penetratin, Kaposi fibroblast growth factor (FGF) signal peptide sequence, integrin β3 signal peptide sequence, polyarginine peptide Args sequence, Guanine rich-molecular transporters, and sweet arrow peptide. CPPs and methods of using them are described, e.g., in Hallbrink et al., “Prediction of cell-penetrating peptides,” Methods Mol. Biol., 2015; 1324:39-58; Ramakrishna et al., “Gene disruption by cell-penetrating peptide-mediated delivery of Cas9 protein and guide RNA,” Genome Res., 2014 June; 24(6):1020-7; and WO 2016205764 A1; each of which is incorporated herein by reference in its entirety.

Various delivery methods for the CRISPR systems described herein are also described, e.g., in U.S. Pat. No. 8,795,965, EP 3009511, WO 2016205764, and WO 2017070605; each of which is incorporated herein by reference in its entirety.

Instead of packaging a single strand (ss)DNA sequence as a vector genome of a AAV particle, systems and methods of packaging an RNA sequence as a vector genome into a AAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of AAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be an RNA sequence for use (although it seems that the RNA coding sequence does not encode something), or an RNA sequence can be produced from the RNA coding sequence, e.g., by RNA processing (although it seems that the RNA coding sequence does not encode something), or a protein can be translated from the RNA coding sequence.

For example, a (e.g., Cas13f, NLS) coding sequence (encoding a (e.g., Cas13f, NLS) polypeptide) covers either a (e.g., Cas13f, NLS) DNA coding sequence from which a (e.g., Cas13f, NLS) polypeptide is expressed (indirectly via transcription and translation) or a (e.g., Cas13f, NLS) RNA coding sequence from which a (e.g., Cas13f, NLS) polypeptide is translated (directly).

For example, a (e.g., gRNA) coding sequence (encoding an RNA (e.g., a gRNA) sequence) covers either a (e.g., gRNA) DNA coding sequence from which an RNA sequence (e.g., a gRNA sequence or array) is transcribed or a (e.g., gRNA) RNA coding sequence (1) which per se is the RNA sequence (e.g., a gRNA sequence or array) for use, or (2) from which a gRNA sequence or array is produced, e.g., by RNA processing.

In some embodiments for RNA AAV vector genomes, 5′-ITR and/or 3′-ITR as DNA packaging signals would be unnecessary and can be omitted, while RNA packaging signals can be introduced.

In some embodiments for AAV RNA vector genomes, promoters to drive transcription of DNA sequences would be unnecessary and can be omitted at least partly.

In some embodiments for AAV RNA vector genomes, polyA signal sequence would be unnecessary and can be omitted, while a polyA tail can be introduced.

Similarly, other DNA elements of AAV DNA vector genomes can be either omitted or replaced with corresponding RNA elements and/or new RNA elements can be introduced, in order to adapt to the strategy of delivering an RNA vector genome by rAAV particles.

### 9. Kits

Another aspect of the disclosure provides a kit, comprising any two or more components of the subject CRISPR-Cas system described herein comprising an engineered Cas13f protein, such as those either substantially lacking or having enhanced collateral activity, such as the Cas13f proteins, derivatives, functional fragments, or the various fusions or adducts thereof, guide RNA/crRNA, complexes thereof, vectors encompassing the same, or host encompassing the same.

In certain embodiments, the kit further comprises an instruction to use the components encompassed therein, and/or instructions for combining with additional components that may be available elsewhere.

In certain embodiments, the kit further comprises one or more nucleotides, such as nucleotide(s) corresponding to those useful to insert the guide RNA coding sequence into a vector and operably linking the coding sequence to one or more control elements of the vector.

In certain embodiments, the kit further comprises one or more buffers that may be used to dissolve any of the components, and/or to provide suitable reaction conditions for one or more of the components.

Such buffers may include one or more of PBS, HEPES, Tris, MOPS, Na₂CO₃, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

The disclosure is further described in the following examples, which do not limit the scope of the disclosure described in the claims.

Further embodiments are illustrated in the following Examples which are given for illustrative purposes only and are not intended to limit the scope of the disclosure.

### EXAMPLES

### Example 1 Engineering of Cas13f for Collateral Activity

This Example demonstrates that by introducing one or more specific amino acid mutations, the spacer sequence-independent collateral cleavage activity (“collateral activity”, “off-target cleavage activity”) of a reference Cas13f polypeptide (wild type, “WT”, SEQ ID NO: 1) can be decreased or increased while maintaining the spacer sequence-specific cleavage activity (“cleavage activity”, “on-target cleavage activity”).

### Designs and Constructions:

A publicly available online tool TASSER was used to predict the 3D structure of reference Cas13f polypeptide, and the predicted structure was visualized with PyMOL as shown in FIG. 1 to predict the position of the various structural domains in 3D.

A one-plasmid mammalian dual-fluorescence reporter system was constructed for detection of the collateral activities of Cas13f mutants as shown in FIG. 2.

The plasmid comprised a Cas13f mutant coding sequence flanked by both 5′ and 3′ SV40 NLS (SEQ ID NO: 5) coding sequences under the regulation of a CAG promoter and a poly A sequence, a EGFP green fluorescent reporter gene (with its RNA transcript as an RNA target for cleavage activity) under the regulation of a SV40 promoter and a poly A sequence, a mCherry red fluorescent reporter gene (with its RNA transcript as RNA target for collateral activity) under the regulation of a SV40 promoter and a poly A sequence, and a sequence encoding a gRNA in 5′-DR sequence (SEQ ID NO: 2)-EGFP-targeting spacer sequence (SEQ ID NO: 6)-DR sequence (SEQ ID NO: 2)-3′ configuration under the regulation of a U6 promoter.

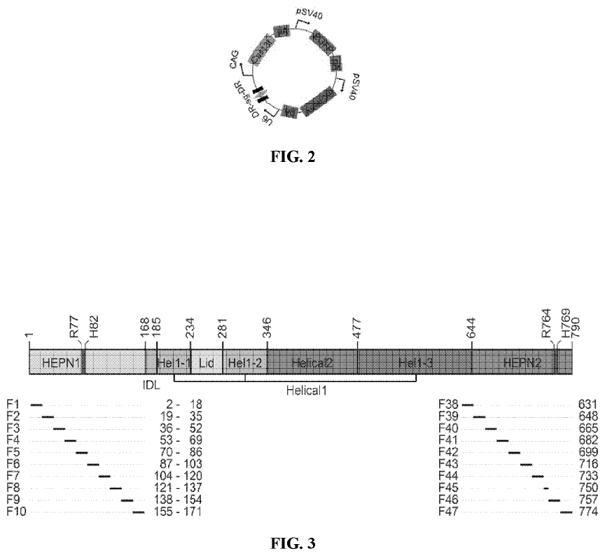

The HEPN1, HEPN2, IDL, and Hell-3 domains of the reference Cas13f polypeptide were chosen for generating a Cas13f mutagenesis library. 20 small segments were selected over those domains (F1-F10 and F38-F47, FIG. 3), each with 17 residues except for F45V1 and F45V2 with 9 residues.

For designing Cas13f mutants, all the non-Ala (A) residues of each segment were substituted with Ala (A) residues in several versions, and all the Ala (A) residues of each segment were substituted with Val (V) residues in several versions. For example, for F1 segment, F1V1-F1V4 mutations were designed. About 4-5 total mutations were introduced into each segment in each version. The Cas13f mutants so generated and the amino acid sequences of the mutated segment are provided in Table 1 below, and the other part of each of the Cas13f mutants is the same as the reference Cas13f polypeptide of SEQ ID NO: 1.

TABLE 1
Design of Cas13f mutants

| Muta- | Amino Acid | Muta- | Amino Acid | Muta- | Amino Acid |
|---|---|---|---|---|---|
| tion | Sequence | tion | Sequence | tion | Sequence |
| F1V1 | AAIELAAEEAAFAFN | F16V4 | KDIFVAEEAFQGNSY | F32V2 | KHGIEAAALRITIDIA |
| | QA | | FA | | K |
| F1V2 | NGAEAKKEEAAFYFA | F17V1 | INAHAAVIAEDELAEL | F32V3 | KAGAENLNARATIDI |
| | AA | | C | | NK |
| F1V3 | NGIALKKAEAAAYAN | F17V2 | IAGHKGAIGEAEAKE | F32V4 | KHGIANLNLAITADA |
| | QA | | LC | | NK |
| F1V4 | NGIELKKEAVVFYFN | F17V3 | ANGAKGVIGEDELKE | F33V1 | ARAAVLARIAIPRAFV |
| | QV | | AA | | A |
| F2V1 | ELALAAIEANIFAAER | F17V4 | INGHKGVAGADALK | F33V2 | SRKAAANRAAIPRGF |
| | R | | ALC | | AK |
| F2V2 | EANAKAAEDAIFDKE | F18V1 | AAFLIANQAANAVEA | F33V3 | SRKVVLNRIAAARGA |
| | RR | | RI | | VK |
| F2V3 | ALNLKAIADNAADK | F18V2 | YAFLIGAADAAKAEG | F33V4 | SAKAVLNAIVIPAGFV |
| | ERR | | RI | | K |
| F2V4 | ELNLKVIEDNIFDKA | F18V3 | YAAAAGNQDANKVE | F34V1 | RHILGWQEAEAVAAA |
| | AA | | GRA | | IR |
| F3V1 | AALLAAPQILAAMEN | F18V4 | YVFLIGNQDVNKVAG | F34V2 | RHIAAWAESEKASKK |
| | FI | | AI | | IR |
| F3V2 | KTAANNPAILAKMEA | F19V1 | AQFLEAFRAANAVQQ | F34V3 | RAALGWQASEKVSK |
| | FI | | VA | | KAR |
| F3V3 | KTLLNNPQAAAKAE | F19V2 | TAFLEKFRNAASAQQ | F34V4 | AHILGAQESAKVSKK |
| | NFA | | AK | | IA |
| F3V4 | KTLLNNAQILVKMAN | F19V3 | TQAAEKFRNANSVAA | F35V1 | EAECEILLAAEAEEL |
| | AI | | VK | | AA |
| F4V1 | FNFRAVAANAAAEID | F19V4 | TQFLAKAANVNSVQ | F35V2 | EAEAEIAASKEYEEA |
| | CL | | QVK | | SK |
| F4V2 | FAFRDATKAAKGEIA | F20V1 | AAEMLAPEAFPANAF | F35V3 | AAACAALLSKEYEEL |
| | CL | | AE | | SK |
| F4V3 | ANFRDVTKNAKGEA | F20V2 | DDEAAKPEYAPAAYF | F35V4 | EVECEILLSKAYAALS |
| | DAA | | AE | | K |
| F4V4 | FNAADVTKNVKGAI | F20V3 | DDAMLKPAYFPANYA | F36V1 | QFFQAADYDAMARI |
| | DCL | | AA | | NAL |
| F5V1 | ALALRELRNFYSHAA | F20V4 | DDEMLKAEYFAVNY | F36V2 | QFFQSKAAAKMTRIA |
| | HA | | FVE | | GL |
| F5V2 | LAKAREARNFYSHY | F21V1 | AAVARIADRVLNRLN | F36V3 | AFFASKDYDKATRIN |
| | VAK | | AA | | GA |
| F5V3 | LLKLAALRNFYSHYV | F21V2 | SGAGRIKARVLARLA | F36V4 | QAAQSKDYDKMTAA |
| | HK | | KA | | NGL |
| F6V1 | RDVRELAAAEAPILE | F21V3 | SGVGRAKDRAANRA | F37V1 | AEANALIALMAVAL |
| | AY | | NKA | | MAQ |
| F6V2 | RAAREASKGEKPILE | F21V4 | SGVGAIKDAVLNALN | F37V2 | YEKAKAIALMAAYL |
| | KA | | KV | | MGA |
| F6V3 | RDVRALSKGAKPAAE | F22V1 | IASNAAAAGEIIAYDA | F37V3 | YEKNKLIAAAAVYAA |
| | KY | | M | | GQ |
| F6V4 | ADVAELSKGEKAILA | F22V2 | IKANKAKKAEIIAAA | F37V4 | YAKNKLAVLMVVYL |
| | KY | | KM | | MGQ |
| F7V1 | YQFAIEAAAAENVAL | F22V3 | AKSAKAKKGEAIAYD | F38V1 | LRILFAEHAALDDIAA |
| | EI | | KA | | T |
| F7V2 | AAFAIESTGSEAAKLE | F22V4 | IKSNKVKKGAIAVYD | F38V2 | ARILFKEHTKLAAITK |
| | I | | KM | | A |
| F7V3 | YQAAAESTGSENVK | F23V1 | REVMAFIAAALPVAE | F38V3 | LRAAFKEATKADDIT |
| | AEA | | AL | | KT |
| F7V4 | YQFVIASTGSANVKL | F23V2 | REAMAFINNSAPADE | F38V4 | LAILAKAHTKLDDAT |
| | AI | | KA | | KT |
| F8V1 | IEAAAWLAAAAALFF | F23V3 | RAVAAAANNSLPVDE | F39V1 | TVDFAIADAVTVAIPF |
| | LC | | KL | | A |
| F8V2 | IENDAWAADAGVAFF | F23V4 | AEVMVFINNSLAVDA | F39V2 | AVAFKISAKVAVKIPF |
| | AA | | KL | | S |
| F8V3 | AANDAWLADAGVLA | F24V1 | APAAYARYLAMVRF | F39V3 | TADFKASDKATAKIPF |
| | ALC | | WDR | | S |
| F8V4 | IENDVALVDVGVLFF | F24V2 | KPKDAKRALGMARF | F39V4 | TVDAKISDKVTVKA |
| | LC | | WAR | | AAS |
| F9V1 | IFLAAAQANALIAGIS | F24V3 | KAKDYKRYAGAVRA | F40V1 | NYPALVYAMAAAYV |
| | G | | WDR | | DNI |
| F9V2 | IFLKKSQAAKLISAIA | F24V4 | KPKDYKAYLGMVAF | F40V2 | NAPSLVATMSSKAVA |
| | A | | ADA | | NI |
| F9V3 | AFAKKSAANKAISGIS | F25V1 | EADNIAREFETAEWA | F40V3 | AYPSLAYTMSSKYAD |
| | G | | AY | | AI |
| F9V4 | IALKKSQVNKLASGA | F25V2 | EKAAIKREFEAKEWS | F40V4 | NYASAVYTASSKYVD |
| | SG | | KA | | NA |
| F10V1 | FARNADAAQPRRNLF | F25V3 | AKDNAKRAAETKEW | F41V1 | GNYGFANADADAPIL |
| | AY | | SKY | | GA |
| F10V2 | FKRADATGQPRRALF | F25V4 | EKDNIKAEFATKAAS | F41V2 | ANYAFSNKAKDKPIL |
| | TA | | KY | | AK |
| F10V3 | AKRNDDTGAPRRNA | F26V1 | LPANFWAAANLERVA | F41V3 | GAAGFSAKDKAKPIL |
| | ATY | | AL | | GK |
| F10V4 | FKANDDTGQAAANL | F26V2 | APSAFWTAKALERAY | F41V4 | GNYGASNKDKDKAA |
| | FTY | | GL | | AGK |
| F11V1 | FAIREAAAVVPEMQA | F26V3 | LPSNAWTAKNAARV | F42V1 | IAAIEAQRMEFIAEVL |
| | HF | | YGA | | A |
| F11V2 | FSIREGYKAAPEMAK | F26V4 | LASNFATVKNLEAVY | F42V2 | IDVIEKARAEFIKEAA |
| | AF | | GL | | G |
| F11V3 | ASAREGYKVVPEAQ | F27V1 | AREAAAELFNALAA | F42V3 | ADVAEKQRMEAAKE |
| | KHA | | AVE | | VLG |
| F11V4 | FSIAAGYKVVAAMQ | F27V2 | AREKNAEAFAKAKA | F42V4 | IDVIAKQAMAFIKAV |
| | KHF | | DAE | | LG |
| F12V1 | LLFALVNHLANQAAA | F27V3 | ARAKNAALANKLKA | F43V1 | FEAYLFDDAIIDAAAF |
| | IE | | DVA | | A |
| F12V2 | LLFSLAAHLSAADDY | F27V4 | VAEKNVELFNKLKVD | F43V2 | FEKALFAAKIIAKSKF |
| | IE | | VE | | A |
| F12V3 | AAFSAVNHASNQDD | F28V1 | AMAERELEAYQAIND | F43V3 | AEKYAFDDKAADKS |
| | YIE | | AA | | KFA |
| F12V4 | LLASLVNALSNQDDY | F28V2 | KMDERELEKAAKIA | F43V4 | FAKYLADDKIIDKSK |
| | AA | | AAK | | AV |
| F13V1 | AAHQPAAIAEALFFH | F28V3 | KADAREAEKYQKAN | F44V1 | AAAAHIAFAEIAEELV |
| | RI | | DAK | | E |
| F13V2 | KAAAPYDIGEGAFFA | F28V4 | KMDEAALAKYQKIN | F44V2 | DTATAASFAEIVEEAA |
| | RI | | DVK | | E |
| F13V3 | KAHQPYDAGEGLAA | F29V1 | ALANLRRLAAAFAVA | F44V3 | DTATHISAAAAVAELV |
| | HRA | | WE | | E |
| F13V4 | KVHQAYDIGAGLFFH | F29V2 | DAAAARRLASDFGA | F44V4 | DTVTHISFVEIVEALV |
| | AI | | KWE | | A |
| F14V1 | AAAFLNIAAILRNMA | F29V3 | DLVNLRRAASDAGV | F45V1 | AAWAADRLA |
| | FY | | KWA | | |
| F14V2 | ASTFAAISGILRAMKF | F29V4 | DLANLAALVSDFGVK | F45V2 | KGADKAAAT |
| | A | | AE | | |
| F14V3 | ASTFLNASGAARNAK | F30V1 | EADWDEYAAQIAAQI | F46V1 | LAALAAARNKALHA |
| | FY | | TD | | EIL |
| F14V4 | VSTALNISGILANMK | F30V2 | EKAWAEYSGQIKKQI | F46V2 | ATKAKDARNKALHG |
| | AY | | AA | | EAA |
| F15V1 | AYQAARLVEQRAELA | F30V3 | EKDWDEASGAAKKA | F46V3 | LTKLKDVRNKALHG |
| | RE | | ITD | | AIL |
| F15V2 | TAASKRLAEARGELK | F30V4 | AKDADAYSGQIKKQ | F47V1 | TGTAFDETAALINEL |
| | RE | | ATD | | AA |
| F15V3 | TYQSKRAVAQRGAA | F31V1 | AQALTIMAQRITAGL | F47V2 | AAASFDEAKSLINEL |
| | KRE | | AA | | KK |
| F15V4 | TYQSKALVEQAGELK | F31V2 | SAKLAIMKQRIAAAL | F47V3 | TGTSFAETKSAIAEAK |
| | AA | | KK | | K |
| F16V1 | AAIFAWEEPFQANAA | F31V3 | SQKATIAKARITAGA | F47V4 | TGTSADATKSLANAL |
| | FE | | KK | | KK |
| F16V2 | KDAAAWEEPFAGAS | F31V4 | SQKLTAMKQAATVG | | |
| | YFE | | LKK | | |
| F16V3 | KDIFAWAAPAQGNSY | F32V1 | AHAIENLNLRIAIAI | | |
| | AE | | NA | | |

### Transfection and Detection:

HEK293T cells were cultured in 24-well tissue culture plates according to standard methods for 12 hours, before the plasmid was transfected into the cells using standard polyethyleneimine (PEI) transfection. The transfected cells were then cultured at 37° C. under 5% CO₂ for about 48 hours. Then the cultured cells were analyzed by flow cytometry.

The cleavage activity of each Cas13f mutant was inversely correlated to the percentage proportion of EGFP positive cells (% EGFP). The lower the % EGFP⁺ is, the higher the cleavage activity would be. The collateral activity of each Cas13f mutant was inversely correlated to the percentage proportion of mCherry positive cells (% mCherry). The higher the % mCherry⁺ is, the lower the collateral activity would be. Dead Cas13f (“dCas13f”, “dead”) (Cas13f mutant with R77A, H82A, R764A, and H769A mutations in HEPN domains based on the reference Cas13f polypeptide of SEQ ID NO: 1) with no cleavage and collateral activities was used as a negative control.

### Results:

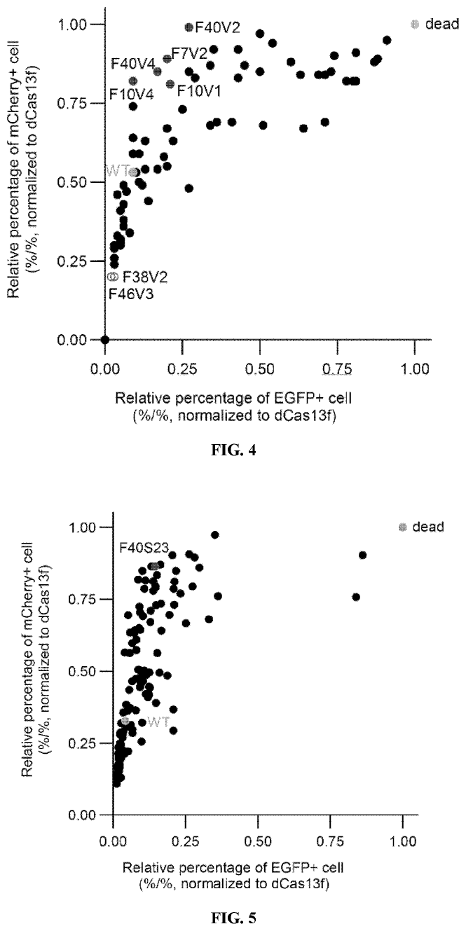

The flow cytometry results (Table 2, FIG. 4) show the cleavage and collateral activities of Cas13f mutants. The Cas13f mutants located at the upper left area of FIG. 4 had low collateral activity (high % mCherry) and high cleavage activity (low % EGFP).

TABLE 2
Averaged cleavage and collateral activities of Cas13f mutants in Table 1 (n = 3)

| | | Collateral | Collateral | | Cleavage | Cleavage |
|---|---|---|---|---|---|---|
| | % | Activity | Activity | % | Activity | Activity |
| Mutation | mCherry | (1-% mCherry) | Relative to WT | EGFP | (1-% EGFP) | Relative to WT |
| dead | 100.00% | 0.00% | — | 100.00% | 0.00% | — |
| WT | 52.78% | 47.22% | 100.00% | 9.44% | 90.56% | 100.00% |
| F2V1 | 84.39% | 15.61% | 33.06% | 62.76% | 37.24% | 41.12% |
| F2V2 | 82.66% | 17.34% | 36.72% | 43.40% | 56.60% | 62.50% |
| F2V3 | 44.29% | 55.71% | 117.98% | 14.45% | 85.55% | 94.47% |
| F2V4 | 62.68% | 37.32% | 79.03% | 21.91% | 78.09% | 86.23% |
| F3V1 | 57.84% | 42.16% | 89.28% | 19.15% | 80.85% | 89.28% |
| F3V2 | 97.49% | 2.51% | 5.32% | 49.88% | 50.12% | 55.34% |
| F3V3 | 72.97% | 27.03% | 57.24% | 25.25% | 74.75% | 82.54% |
| F3V4 | 59.09% | 40.91% | 86.64% | 11.12% | 88.88% | 98.14% |
| F4V1 | 67.83% | 32.17% | 68.13% | 34.02% | 65.98% | 72.86% |
| F4V2 | 94.68% | 5.32% | 11.27% | 90.54% | 9.46% | 10.45% |
| F4V3 | 34.46% | 65.54% | 138.80% | 7.75% | 92.25% | 101.87% |
| F4V4 | 90.46% | 9.54% | 20.20% | 74.16% | 25.84% | 28.53% |
| F5V1 | 93.85% | 6.15% | 13.02% | 53.79% | 46.21% | 51.03% |
| F5V2 | 53.52% | 46.48% | 98.43% | 12.81% | 87.19% | 96.28% |
| F5V3 | 54.05% | 45.95% | 97.31% | 16.88% | 83.12% | 91.78% |
| F6V1 | 83.09% | 16.91% | 35.81% | 28.58% | 71.42% | 78.86% |
| F6V2 | 69.13% | 30.87% | 65.37% | 36.36% | 63.64% | 70.27% |
| F6V3 | 34.26% | 65.74% | 139.22% | 8.29% | 91.71% | 101.27% |
| F6V4 | 62.62% | 37.38% | 79.16% | 12.83% | 87.17% | 96.26% |
| F7V1 | 53.15% | 46.85% | 99.22% | 9.60% | 90.40% | 99.82% |
| F7V2 | 89.15% | 10.85% | 22.98% | 19.56% | 80.44% | 88.83% |
| F7V3 | 68.61% | 31.39% | 66.48% | 41.22% | 58.78% | 64.91% |
| F7V4 | 47.94% | 52.06% | 110.25% | 27.48% | 72.52% | 80.08% |
| F7V4 | 83.93% | 16.07% | 34.03% | 69.18% | 30.82% | 34.03% |
| F8V1 | 81.71% | 18.29% | 38.73% | 79.74% | 20.26% | 22.37% |
| F8V2 | 82.28% | 17.72% | 37.53% | 78.36% | 21.64% | 23.90% |
| F8V3 | 81.80% | 18.20% | 38.54% | 81.01% | 18.99% | 20.97% |
| F8V4 | 31.62% | 68.38% | 144.81% | 4.94% | 95.06% | 104.97% |
| F9V1 | 86.56% | 13.44% | 28.46% | 45.49% | 54.51% | 60.19% |
| F9V2 | 49.51% | 50.49% | 106.93% | 10.51% | 89.49% | 98.82% |
| F9V3 | 69.49% | 30.51% | 64.61% | 71.16% | 28.84% | 31.85% |
| F9V4 | 66.77% | 33.23% | 70.37% | 63.70% | 36.30% | 40.08% |
| F10V1 | 81.31% | 18.69% | 39.58% | 21.23% | 78.77% | 86.98% |
| F10V2 | 31.65% | 68.35% | 144.75% | 4.70% | 95.30% | 105.23% |
| F10V3 | 83.60% | 16.40% | 34.73% | 71.23% | 28.77% | 31.77% |
| F10V4 | 82.15% | 17.85% | 37.80% | 9.29% | 90.71% | 100.17% |
| F38V1 | 32.61% | 67.39% | 142.71% | 3.81% | 96.19% | 106.22% |
| F38V2 | 20.31% | 79.69% | 168.76% | 3.50% | 96.50% | 106.56% |
| F38V3 | 30.78% | 69.22% | 146.59% | 5.26% | 94.74% | 104.62% |
| F38V4 | 58.60% | 41.40% | 87.67% | 9.04% | 90.96% | 100.44% |
| F39V1 | 47.31% | 52.69% | 111.58% | 7.36% | 92.64% | 102.30% |
| F39V2 | 46.39% | 53.61% | 113.53% | 3.86% | 96.14% | 106.16% |
| F39V3 | 92.12% | 7.88% | 16.69% | 35.47% | 64.53% | 71.26% |
| F39V4 | 91.68% | 8.32% | 17.62% | 42.72% | 57.28% | 63.25% |
| F40V1 | 64.40% | 35.60% | 75.39% | 8.56% | 91.44% | 100.97% |
| F40V2 | 98.57% | 1.43% | 3.03% | 27.11% | 72.89% | 80.49% |
| F40V3 | 26.44% | 73.56% | 155.78% | 3.41% | 96.59% | 106.66% |
| F40V4 | 85.24% | 14.76% | 31.26% | 16.98% | 83.02% | 91.67% |
| F41V1 | 52.81% | 47.19% | 99.94% | 9.63% | 90.37% | 99.79% |
| F41V2 | 35.67% | 64.33% | 136.23% | 6.44% | 93.56% | 103.31% |
| F41V3 | 74.46% | 25.54% | 54.09% | 8.86% | 91.14% | 100.64% |
| F41V4 | 87.26% | 12.74% | 26.98% | 34.35% | 65.65% | 72.49% |
| F42V1 | 23.98% | 76.02% | 160.99% | 3.06% | 96.94% | 107.05% |
| F42V2 | 68.10% | 31.90% | 67.56% | 51.06% | 48.94% | 54.04% |
| F42V3 | 88.21% | 11.79% | 24.97% | 87.02% | 12.98% | 14.33% |
| F42V4 | 67.18% | 32.82% | 69.50% | 20.16% | 79.84% | 88.16% |
| F43V1 | 55.08% | 44.92% | 95.13% | 19.99% | 80.01% | 88.35% |
| F43V2 | 29.09% | 70.91% | 150.17% | 2.93% | 97.07% | 107.19% |
| F43V3 | 85.38% | 14.62% | 30.96% | 73.31% | 26.69% | 29.47% |
| F43V4 | 91.33% | 8.67% | 18.36% | 81.46% | 18.54% | 20.47% |
| F44V1 | 49.36% | 50.64% | 107.24% | 5.85% | 94.15% | 103.96% |
| F44V2 | 85.19% | 14.81% | 31.36% | 27.28% | 72.72% | 80.30% |
| F44V3 | 88.13% | 11.87% | 25.14% | 59.60% | 40.40% | 44.61% |
| F44V3 | 94.20% | 5.80% | 12.28% | 88.56% | 11.44% | 12.63% |
| F44V4 | 28.71% | 71.29% | 150.97% | 2.62% | 97.38% | 107.53% |
| F45V1 | 49.07% | 50.93% | 107.86% | 12.29% | 87.71% | 96.85% |
| F45V2 | 30.45% | 69.55% | 147.29% | 4.59% | 95.41% | 105.36% |
| F46V1 | 41.39% | 58.61% | 124.12% | 4.77% | 95.23% | 105.16% |
| F46V2 | 88.99% | 11.01% | 23.32% | 87.97% | 12.03% | 13.28% |
| F46V3 | 20.17% | 79.83% | 169.06% | 1.99% | 98.01% | 108.23% |
| F47V1 | 85.00% | 15.00% | 31.77% | 49.65% | 50.35% | 55.60% |
| F47V1 | 43.31% | 56.69% | 120.06% | 6.02% | 93.98% | 103.78% |
| F47V2 | 29.73% | 70.27% | 148.81% | 3.47% | 96.53% | 106.59% |
| F47V3 | 37.90% | 62.10% | 131.51% | 6.07% | 93.93% | 103.72% |
| F47V4 | 83.56% | 16.44% | 34.82% | 70.86% | 29.14% | 32.18% |

It was found that the Cas13f mutants with mutation in F7, F10, F40, F38, or F46, specially F7V2, F10V1, F10V4, F40V2, F40V4, F38V2, or F46V3, exhibited relatively low % EGFP but much higher or lower % mCherry, indicating that these mutants retained a high cleavage activity but greatly reduced or enhanced collateral activity.

A second round of mutagenesis study in or nearby these regions (F10V1, F10V4, F38V2, F40V2, F40V4, F46V1, and F46V3) of these mutants was conducted by generating a number of additional mutants with single or multiple (e.g., double, triple, or quadruple) combination mutations. The sequences of the mutated segments of these mutants are listed in Table 3 below, and their cleavage and collateral activities are listed in Table 4 below and FIG. 5.

TABLE 3
Design of Cas13f mutants

| | Amino Acid | | Amino Acid | | Amino Acid |
|---|---|---|---|---|---|
| Mutation | Sequence | Mutation | Sequence | Mutation | Sequence |
| F10S1 | AKRNDDTGQP | F40S7 | NYPSLVATMS | F10S33 | FARNADTGQP |
| | RRNLFTY | | SKYVDNI | | RRNLFTY |
| F10S2 | FARNDDTGQP | F40S8 | NYPSLVYAMS | F10S34 | FARNDDAGQP |
| | RRNLFTY | | SKYVDNI | | RRNLFTY |
| F10S3 | FKANDDTGQP | F40S9 | NYPSLVYTAS | F10S35 | FARNDDTAQP |
| | RRNLFTY | | SKYVDNI | | RRNLFTY |
| F10S4 | FKRADDTGQP | F40S10 | NYPSLVYTMA | F10S36 | FARNDDTGQP |
| | RRNLFTY | | SKYVDNI | | RRNLFAY |
| F10S5 | FKRNADTGQP | F40S11 | NYPSLVYTMS | F10S37 | FKRNADAGQP |
| | RRNLFTY | | AKYVDNI | | RRNLFTY |
| F10S6 | FKRNDATGQP | F40S12 | NYPSLVYTMS | F10S38 | FKRNADTAQP |
| | RRNLFTY | | SAYVDNI | | RRNLFTY |
| F10S7 | FKRNDDAGQP | F40S13 | NYPSLVYTMS | F10S39 | FKRNADTGQP |
| | RRNLFTY | | SKAVDNI | | RRNLFAY |
| F10S8 | FKRNDDTAQP | F40S14 | NYPSLVYTMS | F10S40 | FKRNDDAAQP |
| | RRNLFTY | | SKYADNI | | RRNLFTY |
| F10S9 | FKRNDDTGAP | F40S15 | NYPSLVYTMS | F10S41 | FKRNDDAGQP |
| | RRNLFTY | | SKYVANI | | RRNLFAY |
| F10S10 | FKRNDDTGQA | F40S16 | NYPSLVYTMS | F10S42 | FKRNDDTAQP |
| | RRNLFTY | | SKYVDAI | | RRNLFAY |
| F10S11 | FKRNDDTGQP | F40S17 | NYPSLVYTMS | F10S43 | FKANDDTGQA |
| | ARNLFTY | | SKYVDNA | | ARNLFTY |
| F10S12 | FKRNDDTGQP | F46S1 | ATKLKDARNK | F10S44 | FKANDDTGQA |
| | RANLFTY | | ALHGEIL | | RANLFTY |
| F10S13 | FKRNDDTGQP | F46S2 | LAKLKDARNK | F10S45 | FKANDDTGQP |
| | RRALFTY | | ALHGEIL | | AANLFTY |
| F10S14 | FKRNDDTGQP | F46S3 | LTALKDARNK | F10S46 | FKRNDDTGQA |
| | RRNAFTY | | ALHGEIL | | AANLFTY |
| F10S15 | FKRNDDTGQP | F46S4 | LTKAKDARNK | F10S47 | FKANDDTGQA |
| | RRNLATY | | ALHGEIL | | RRNLFTY |
| F10S16 | FKRNDDTGQP | F46S5 | LTKLADARNK | F10S48 | FKANDDTGQP |
| | RRNLFAY | | ALHGEIL | | ARNLFTY |
| F10S17 | FKRNDDTGQP | F46S6 | LTKLKAARNK | F10S49 | FKANDDTGQP |
| | RRNLFTA | | ALHGEIL | | RANLFTY |
| F38S1 | ARILFKEHTK | F46S7 | LTKLKDVRNK | F10S50 | FKRNDDTGQA |
| | LDDITKT | | ALHGEIL | | ARNLFTY |
| F38S2 | LAILFKEHTK | F46S10 | LTKLKDARNA | F10S51 | FKRNDDTGQA |
| | LDDITKT | | ALHGEIL | | RANLFTY |
| F38S3 | LRALFKEHTK | F46S11 | LTKLKDARNK | F10S52 | FKRNDDTGQP |
| | LDDITKT | | VLHGEIL | | AANLFTY |
| F38S4 | LRIAFKEHTK | F46S12 | LTKLKDARNK | F40S18 | NAPSLVATMS |
| | LDDITKT | | AAHGEIL | | SKAVDNI |
| F38S5 | LRI LAKEHTKLDD | F46S14 | LTKLKDARNK | F40S19 | NAPSLVATMS |
| | ITKT | | ALHAEIL | | SKYVANI |
| F38S6 | LRILFAEHTK | F46S15 | LTKLKDARNK | F40S20 | NAPSLVYTMS |
| | LDDITKT | | ALHGAIL | | SKAVANI |
| F38S7 | LRILFKAHTK | F46S16 | LTKLKDARNK | F40S21 | NYPSLVATMS |
| | LDDITKT | | ALHGEAL | | SKAVANI |
| F38S8 | LRILFKEATK | F46S17 | LTKLKDARNK | F40S22 | NAPSLVATMS |
| | LDDITKT | | ALHGEIA | | SKYVDNI |
| F38S9 | LRILFKEHAK | F10S18 | FARNADAAQP | F40S23 | NAPSLVYTMS |
| | LDDITKT | | RRNLFTY | | SKAVDNI |
| F38S10 | LRILFKEHTA | F10S19 | FARNADAGQP | F40S24 | NAPSLVYTMS |
| | LDDITKT | | RRNLFAY | | SKYVANI |
| F38S11 | LRILFKEHTK | F10S20 | FARNADTAQP | F40S25 | NYPSLVATMS |
| | ADDITKT | | RRNLFAY | | SKAVDNI |
| F38S12 | LRILFKEHTK | F10S21 | FARNDDAAQP | F40S26 | NYPSLVATMS |
| | LADITKT | | RRNLFAY | | SKYVANI |
| F38S13 | LRILFKEHTK | F10S22 | FKRNADAAQP | F40S27 | NYPSLVYTMS |
| | LDAITKT | | RRNLFAY | | SKAVANI |
| F38S14 | LRILFKEHTK | F10S23 | FARNADAGQP | F40S28 | NYASAVYTAS |
| | LDDATKT | | RRNLFTY | | SKYVDNI |
| F38S15 | LRILFKEHTK | F10S24 | FARNADTAQP | F40S29 | NYASAVYTMS |
| | LDDIAKT | | RRNLFTY | | SKYVDNA |
| F38S16 | LRILFKEHTK | F10S25 | FARNADTGQP | F40S30 | NYASLVYTAS |
| | LDDITAT | | RRNLFAY | | SKYVDNA |
| F38S17 | LRILFKEHTK | F10S26 | FARNDDAAQP | F40S31 | NYPSAVYTAS |
| | LDDITKA | | RRNLFTY | | SKYVDNA |
| F40S1 | AYPSLVYTMS | F10S27 | FARNDDAGQP | F40S32 | NYASAVYTMS |
| | SKYVDNI | | RRNLFAY | | SKYVDNI |
| F40S2 | NAPSLVYTMS | F10S28 | FARNDDTAQP | F40S33 | NYASLVYTAS |
| | SKYVDNI | | RRNLFAY | | SKYVDNI |
| F40S3 | NYASLVYTMS | F10S29 | FKRNADAAQP | F40S34 | NYASLVYTMS |
| | SKYVDNI | | RRNLFTY | | SKYVDNA |
| F40S4 | NYPALVYTMS | F10S30 | FKRNADAGQP | F40S35 | NYPSAVYTAS |
| | SKYVDNI | | RRNLFAY | | SKYVDNI |
| F40S5 | NYPSAVYTMS | F10S31 | FKRNADTAQP | F40S36 | NYPSAVYTMS |
| | SKYVDNI | | RRNLFAY | | SKYVDNA |
| F40S6 | NYPSLAYTMS | F10S32 | FKRNDDAAQP | F40S37 | NYPSLVYTAS |
| | SKYVDNI | | RRNLFAY | | SKYVDNA |

TABLE 4
Averaged cleavage and collateral activities of Cas13f mutants in Table 3 (n = 3)

| | | Collateral | Collateral | | Cleavage | Cleavage |
|---|---|---|---|---|---|---|
| | % | Activity | Activity | % | Activity | Activity |
| Mutation | mCherry | (1-% mCherry) | Relative to WT | EGFP | (1-% EGFP) | Relative to WT |
| dead | 100.00% | 0.00% | — | 100.00% | 0.00% | — |
| WT | 32.81% | 67.19% | 100.00% | 4.30% | 95.70% | 100.00% |
| F10V1 | 76.12% | 23.88% | 35.54% | 36.23% | 63.77% | 66.64% |
| F10V4 | 69.16% | 30.84% | 45.90% | 10.36% | 89.64% | 93.67% |
| F38V2 | 22.17% | 77.83% | 115.84% | 3.30% | 96.70% | 101.04% |
| F40V2 | 97.30% | 2.70% | 4.02% | 35.12% | 64.88% | 67.80% |
| F40V4 | 73.51% | 26.49% | 39.43% | 16.53% | 83.47% | 87.22% |
| F46V1 | 46.65% | 53.35% | 79.40% | 10.38% | 89.62% | 93.65% |
| F46V3 | 14.19% | 85.81% | 127.71% | 1.34% | 98.66% | 103.09% |
| F38S2 | 21.31% | 78.69% | 117.12% | 2.99% | 97.01% | 101.37% |
| F38S3 | 31.50% | 68.50% | 101.95% | 4.53% | 95.47% | 99.76% |
| F38S4 | 16.00% | 84.00% | 125.02% | 2.16% | 97.84% | 102.24% |
| F38S5 | 21.33% | 78.67% | 117.09% | 2.85% | 97.15% | 101.52% |
| F38S6 | 20.66% | 79.34% | 118.08% | 2.34% | 97.66% | 102.05% |
| F38S7 | 17.70% | 82.30% | 122.49% | 2.29% | 97.71% | 102.10% |
| F38S8 | 19.61% | 80.39% | 119.65% | 2.02% | 97.98% | 102.38% |
| F38S9 | 19.97% | 80.03% | 119.11% | 2.58% | 97.42% | 101.80% |
| F38S10 | 13.89% | 86.11% | 128.16% | 1.85% | 98.15% | 102.56% |
| F38S11 | 17.80% | 82.20% | 122.34% | 2.24% | 97.76% | 102.15% |
| F38S12 | 13.53% | 86.47% | 128.69% | 1.70% | 98.30% | 102.72% |
| F38S13 | 19.83% | 80.17% | 119.32% | 2.78% | 97.22% | 101.59% |
| F38S15 | 17.22% | 82.78% | 123.20% | 1.78% | 98.22% | 102.63% |
| F38S16 | 19.40% | 80.60% | 119.96% | 2.27% | 97.73% | 102.12% |
| F38S17 | 20.16% | 79.84% | 118.83% | 2.03% | 97.97% | 102.37% |
| F40S1 | 23.02% | 76.98% | 114.57% | 2.57% | 97.43% | 101.81% |
| F40S2 | 21.38% | 78.62% | 117.01% | 1.89% | 98.11% | 102.52% |
| F40S3 | 17.89% | 82.11% | 122.21% | 2.01% | 97.99% | 102.39% |
| F40S4 | 16.33% | 83.67% | 124.53% | 1.98% | 98.02% | 102.42% |
| F40S5 | 22.66% | 77.34% | 115.11% | 3.32% | 96.68% | 101.02% |
| F40S6 | 20.38% | 79.62% | 118.50% | 2.41% | 97.59% | 101.97% |
| F40S7 | 63.27% | 36.73% | 54.67% | 7.29% | 92.71% | 96.88% |
| F40S8 | 22.95% | 77.05% | 114.67% | 2.73% | 97.27% | 101.64% |
| F40S9 | 50.53% | 49.47% | 73.63% | 8.76% | 91.24% | 95.34% |
| F40S11 | 50.24% | 49.76% | 74.06% | 9.66% | 90.34% | 94.40% |
| F40S12 | 48.81% | 51.19% | 76.19% | 11.61% | 88.39% | 92.36% |
| F40S13 | 48.52% | 51.48% | 76.62% | 18.70% | 81.30% | 84.95% |
| F40S14 | 44.60% | 55.40% | 82.45% | 12.38% | 87.62% | 91.56% |
| F40S15 | 32.20% | 67.80% | 100.91% | 10.02% | 89.98% | 94.02% |
| F40S16 | 25.60% | 74.40% | 110.73% | 9.79% | 90.21% | 94.26% |
| F40S17 | 49.58% | 50.42% | 75.04% | 12.54% | 87.46% | 91.39% |
| F40S18 | 29.38% | 70.62% | 105.10% | 20.85% | 79.15% | 82.71% |
| F40S19 | 39.01% | 60.99% | 90.77% | 14.82% | 85.18% | 89.01% |
| F40S20 | 36.77% | 63.23% | 94.11% | 20.81% | 79.19% | 82.75% |
| F40S21 | 90.66% | 9.34% | 13.90% | 26.23% | 73.77% | 77.08% |
| F40S22 | 81.19% | 18.81% | 28.00% | 13.85% | 86.15% | 90.02% |
| F40S25 | 68.11% | 31.89% | 47.46% | 33.03% | 66.97% | 69.98% |
| F40S26 | 87.07% | 12.93% | 19.24% | 16.33% | 83.67% | 87.43% |
| F40S28 | 59.78% | 40.22% | 59.86% | 6.65% | 93.35% | 97.54% |
| F40S29 | 50.32% | 49.68% | 73.94% | 10.80% | 89.20% | 93.21% |
| F40S30 | 64.16% | 35.84% | 53.34% | 16.69% | 83.31% | 87.05% |
| F40S31 | 85.97% | 14.03% | 20.88% | 29.81% | 70.19% | 73.34% |
| F40S32 | 46.55% | 53.45% | 79.55% | 6.65% | 93.35% | 97.54% |
| F40S33 | 37.23% | 62.77% | 93.42% | 5.87% | 94.13% | 98.36% |
| F40S34 | 30.51% | 69.49% | 103.42% | 4.45% | 95.55% | 99.84% |
| F40S35 | 57.38% | 42.62% | 63.43% | 8.02% | 91.98% | 96.11% |
| F40S36 | 84.91% | 15.09% | 22.46% | 21.74% | 78.26% | 81.78% |
| F40S37 | 67.07% | 32.93% | 49.01% | 12.95% | 87.05% | 90.96% |
| F46S1 | 21.37% | 78.63% | 117.03% | 4.13% | 95.87% | 100.18% |
| F46S2 | 75.80% | 24.20% | 36.02% | 83.89% | 16.11% | 16.83% |
| F46S4 | 22.22% | 77.78% | 115.76% | 5.19% | 94.81% | 99.07% |
| F46S5 | 35.62% | 64.38% | 95.82% | 3.54% | 96.46% | 100.79% |
| F46S6 | 15.32% | 84.68% | 126.03% | 2.10% | 97.90% | 102.30% |
| F46S7 | 21.88% | 78.12% | 116.27% | 2.41% | 97.59% | 101.97% |
| F46S10 | 21.36% | 78.64% | 117.04% | 3.09% | 96.91% | 101.26% |
| F46S11 | 47.44% | 52.56% | 78.23% | 8.00% | 92.00% | 96.13% |
| F46S12 | 28.56% | 71.44% | 106.33% | 6.74% | 93.26% | 97.45% |
| F46S14 | 16.75% | 83.25% | 123.90% | 2.37% | 97.63% | 102.02% |
| F46S15 | 11.06% | 88.94% | 132.37% | 1.31% | 98.69% | 103.12% |
| F10S1 | 47.87% | 52.13% | 77.59% | 9.32% | 90.68% | 94.75% |
| F10S2 | 60.95% | 39.05% | 58.12% | 8.08% | 91.92% | 96.05% |
| F10S3 | 28.01% | 71.99% | 107.14% | 2.46% | 97.54% | 101.92% |
| F10S4 | 13.75% | 86.25% | 128.37% | 1.77% | 98.23% | 102.64% |
| F10S5 | 13.10% | 86.90% | 129.33% | 2.70% | 97.30% | 101.67% |
| F10S6 | 13.06% | 86.94% | 129.39% | 1.48% | 98.52% | 102.95% |
| F10S8 | 28.72% | 71.28% | 106.09% | 2.61% | 97.39% | 101.77% |
| F10S9 | 16.59% | 83.41% | 124.14% | 1.48% | 98.52% | 102.95% |
| F10S10 | 23.55% | 76.45% | 113.78% | 1.97% | 98.03% | 102.43% |
| F10S12 | 64.24% | 35.76% | 53.22% | 7.51% | 92.49% | 96.65% |
| F10S13 | 29.06% | 70.94% | 105.58% | 3.52% | 96.48% | 100.82% |
| F10S17 | 29.73% | 70.27% | 104.58% | 6.75% | 93.25% | 97.44% |
| F10S18 | 70.99% | 29.01% | 43.18% | 13.04% | 86.96% | 90.87% |
| F10S19 | 79.44% | 20.56% | 30.60% | 27.44% | 72.56% | 75.82% |
| F10S21 | 76.93% | 23.07% | 34.34% | 23.26% | 76.74% | 80.19% |
| F10S22 | 44.22% | 55.78% | 83.02% | 12.72% | 87.28% | 91.20% |
| F10S23 | 73.04% | 26.96% | 40.13% | 14.92% | 85.08% | 88.90% |
| F10S24 | 77.93% | 22.07% | 32.85% | 13.93% | 86.07% | 89.94% |
| F10S26 | 79.52% | 20.48% | 30.48% | 14.58% | 85.42% | 89.26% |
| F10S27 | 78.63% | 21.37% | 31.81% | 20.90% | 79.10% | 82.65% |
| F10S28 | 73.15% | 26.85% | 39.96% | 21.12% | 78.88% | 82.42% |
| F10S29 | 36.34% | 63.66% | 94.75% | 5.08% | 94.92% | 99.18% |
| F10S30 | 41.86% | 58.14% | 86.53% | 12.43% | 87.57% | 91.50% |
| F10S31 | 56.32% | 43.68% | 65.01% | 15.32% | 84.68% | 88.48% |
| F10S32 | 31.35% | 68.65% | 102.17% | 6.19% | 93.81% | 98.03% |
| F10S33 | 83.36% | 16.64% | 24.77% | 15.15% | 84.85% | 88.66% |
| F10S34 | 78.65% | 21.35% | 31.78% | 10.81% | 89.19% | 93.20% |
| F10S35 | 81.50% | 18.50% | 27.53% | 11.26% | 88.74% | 92.73% |
| F10S36 | 81.09% | 18.91% | 28.14% | 21.22% | 78.78% | 82.32% |
| F10S37 | 32.27% | 67.73% | 100.80% | 4.32% | 95.68% | 99.98% |
| F10S38 | 44.43% | 55.57% | 82.71% | 9.31% | 90.69% | 94.76% |
| F10S39 | 49.52% | 50.48% | 75.13% | 16.10% | 83.90% | 87.67% |
| F10S40 | 32.02% | 67.98% | 101.18% | 2.82% | 97.18% | 101.55% |
| F10S41 | 36.42% | 63.58% | 94.63% | 7.83% | 92.17% | 96.31% |
| F10S42 | 45.67% | 54.33% | 80.86% | 9.60% | 90.40% | 94.46% |
| F10S43 | 63.40% | 36.60% | 54.47% | 5.93% | 94.07% | 98.30% |
| F10S44 | 70.46% | 29.54% | 43.96% | 9.39% | 90.61% | 94.68% |
| F10S45 | 90.27% | 9.73% | 14.48% | 20.48% | 79.52% | 83.09% |
| F10S46 | 79.02% | 20.98% | 31.22% | 14.62% | 85.38% | 89.22% |
| F10S47 | 56.27% | 43.73% | 65.08% | 5.79% | 94.21% | 98.44% |
| F10S48 | 84.92% | 15.08% | 22.44% | 10.15% | 89.85% | 93.89% |
| F10S49 | 86.39% | 13.61% | 20.26% | 13.26% | 86.74% | 90.64% |
| F10S50 | 72.44% | 27.56% | 41.02% | 9.16% | 90.84% | 94.92% |
| F10S51 | 64.41% | 35.59% | 52.97% | 9.48% | 90.52% | 94.59% |
| F10S52 | 69.55% | 30.45% | 45.32% | 19.42% | 80.58% | 84.20% |
| F10S7 | 24.93% | 75.07% | 111.73% | 2.43% | 97.57% | 101.95% |
| F10S11 | 65.01% | 34.99% | 52.08% | 9.00% | 91.00% | 95.09% |
| F10S14 | 27.91% | 72.09% | 107.29% | 3.36% | 96.64% | 100.98% |
| F10S15 | 42.10% | 57.90% | 86.17% | 11.36% | 88.64% | 92.62% |
| F10S16 | 41.00% | 59.00% | 87.81% | 11.98% | 88.02% | 91.97% |
| F10S20 | 66.74% | 33.26% | 49.50% | 25.15% | 74.85% | 78.21% |
| F10S25 | 89.51% | 10.49% | 15.61% | 28.09% | 71.91% | 75.14% |
| F10S43 | 69.47% | 30.53% | 45.44% | 5.15% | 94.85% | 99.11% |
| F38S1 | 21.47% | 78.53% | 116.88% | 1.91% | 98.09% | 102.50% |
| F38S13 | 24.62% | 75.38% | 112.19% | 2.72% | 97.28% | 101.65% |
| F40S10 | 38.43% | 61.57% | 91.64% | 4.55% | 95.45% | 99.74% |
| F40S23 | 86.38% | 13.62% | 20.27% | 14.48% | 85.52% | 89.36% |
| F40S24 | 56.52% | 43.48% | 64.71% | 4.14% | 95.86% | 100.17% |
| F40S27 | 81.81% | 18.19% | 27.07% | 8.77% | 91.23% | 95.33% |
| F46S2 | 24.44% | 75.56% | 112.46% | 2.58% | 97.42% | 101.80% |
| F46S3 | 90.32% | 9.68% | 14.41% | 86.15% | 13.85% | 14.47% |
| F46S16 | 43.54% | 56.46% | 84.03% | 5.55% | 94.45% | 98.69% |
| F46S17 | 27.08% | 72.92% | 108.53% | 3.32% | 96.68% | 101.02% |

TABLE 5
Averaged cleavage and collateral activities of
some Cas13f mutants from Tables 2 and 4 (n = 3)

| | | Collateral | Collateral | | Cleavage | Cleavage |
|---|---|---|---|---|---|---|
| | % | Activity | Activity | % | Activity | Activity |
| Mutation | mCherry | (1-% mCherry) | Relative to WT | EGFP | (1-% EGFP) | Relative to WT |
| F7V2 | 89.15% | 10.85% | 22.98% | 19.56% | 80.44% | 88.83% |
| F10V1 | 81.31% | 18.69% | 39.58% | 21.23% | 78.77% | 86.98% |
| F10V4 | 82.15% | 17.85% | 37.80% | 9.29% | 90.71% | 100.17% |
| F40V4 | 85.24% | 14.76% | 31.26% | 16.98% | 83.02% | 91.67% |
| F40S22 | 81.19% | 18.81% | 28.00% | 13.85% | 86.15% | 90.02% |
| F40S26 | 87.07% | 12.93% | 19.24% | 16.33% | 83.67% | 87.43% |
| F40S36 | 84.91% | 15.09% | 22.46% | 21.74% | 78.26% | 81.78% |
| F10S21 | 76.93% | 23.07% | 34.34% | 23.26% | 76.74% | 80.19% |
| F10S24 | 77.93% | 22.07% | 32.85% | 13.93% | 86.07% | 89.94% |
| F10S26 | 79.52% | 20.48% | 30.48% | 14.58% | 85.42% | 89.26% |
| F10S27 | 78.63% | 21.37% | 31.81% | 20.90% | 79.10% | 82.65% |
| F10S33 | 83.36% | 16.64% | 24.77% | 15.15% | 84.85% | 88.66% |
| F10S34 | 78.65% | 21.35% | 31.78% | 10.81% | 89.19% | 93.20% |
| F10S35 | 81.50% | 18.50% | 27.53% | 11.26% | 88.74% | 92.73% |
| F10S36 | 81.09% | 18.91% | 28.14% | 21.22% | 78.78% | 82.32% |
| F10S45 | 90.27% | 9.73% | 14.48% | 20.48% | 79.52% | 83.09% |
| F10S46 | 79.02% | 20.98% | 31.22% | 14.62% | 85.38% | 89.22% |
| F10S48 | 84.92% | 15.08% | 22.44% | 10.15% | 89.85% | 93.89% |
| F10S49 | 86.39% | 13.61% | 20.26% | 13.26% | 86.74% | 90.64% |
| F40S23 | 86.38% | 13.62% | 20.27% | 14.48% | 85.52% | 89.36% |
| F40S27 | 81.81% | 18.19% | 27.07% | 8.77% | 91.23% | 95.33% |

Overall, the Cas13f mutants in Table 5 exhibited both a low collateral activity (e.g., <25% collateral activity represented as >750% mCherry⁺ cells) and a high cleavage activity (e.g., >75% cleavage activity represented as <25% EGFP⁺ cells), including F40S23 (containing Y666A & Y677A mutations, which Cas13f mutant was designated as “Cas13f v2” of full length of SEQ ID NO: 3).

Some other Cas13f mutants retained a high cleavage activity (e.g., >75% cleavage activity represented as <25% EGFP⁺ cells) but also a high collateral activity (e.g., >75% collateral activity represented as <25% mCherry⁺ cells). Such Cas13f mutants maybe useful for detection methods such as SHERLOCK relying on both cleavage and collateral activities.

### Example 2 Engineering of Cas13f for Increased Cleavage Activity

Cas13f mutants had been screened for a low spacer sequence-independent collateral cleavage activity (“collateral activity”, “off-target cleavage activity”) in Example 1. In order to further improve the spacer sequence-specific cleavage activity (“cleavage activity”, “on-target cleavage activity”) while ensuring no or low collateral activity, one or more of mutations (Table 6) was further introduced into mutant F40S23 (Cas13f-Y666A, Y677A, or designated as Cas13f v2, SEQ ID NO: 3) developed in Example 1.

This Example demonstrates that by introducing one or more specific amino acid mutations, the cleavage activity of Cas13f v2 can be increased.

TABLE 6
Available mutations for introduction into Cas13f v2

| | Corresponding mutation |
|---|---|
| Mutation | name in Example 1 |
| D160A | F10S6 |
| Q163A | F10S9 |
| D642A | F38S12 |
| L631A | F38S1 |
| P667A | F40S3 |
| H638A | F38S8 |
| T647A | F38S17 |
| D762A | F46S6 |
| L634A | F38S4 |
| L641A | F38S11 |
| V670A | F40S6 |
| A763V | F46S7 |
| T161A | F10S7 |

### Designs and Constructions:

A two-plasmid mammalian fluorescence reporter system was constructed for detection of the cleavage activities of Cas13f mutants.

One plasmid comprised a ATXN2 cDNA coding sequence (with its RNA transcript as a cleavage target) followed by a p2A (self-cleaving peptide) and an EGFP reporter gene (SEQ ID NO: 7) under the regulation of SV40 promoter and a poly A sequence, as shown in FIG. 6. EGFP mRNA was transcribed together with the ATXN2 RNA transcript from the plasmid to form a chimeric transcript. When the ATXN2 RNA transcript as a part of the chimeric transcript was cleaved by a ATXN2-targeting gRNA guided Cas13f mutant, the EGFR mRNA as another part of the chimeric transcript would also be gradually degraded due to, e.g., overall RNA instability, leading to reduced fluorescent intensity of EGFP (Green).

The other plasmid comprised a Cas13f mutant coding sequence flanked by both 5′ and 3′ SV40 NLS (SEQ ID NO: 5) coding sequence under the regulation of a Cbh promoter and a poly A sequence, a sequence encoding a gRNA in 5′-DR sequence (SEQ ID NO: 2)-AXTN2-targeting spacer sequence (SEQ ID NO: 8)-DR sequence (SEQ ID NO: 2)-3′ configuration under the regulation of a U6 promoter and a mCherry reporter gene (with its RNA transcript as a collateral cleavage target) under the regulation of a SV40 promoter and a poly A sequence. As a negative control, a non-targeting spacer sequence (“NT”, SEQ ID NO: 9) was used in place of the AXTN2-targeting spacer sequence (SEQ ID NO: 8). In the case that the Cas13f mutant retained collateral activity, the mCherry RNA transcript may be cleaved, leading to reduced fluorescent intensity of mCherry (Red).

A similar pair of plasmids was constructed with Rho cDNA coding sequence followed by a p2A (self-cleaving peptide) and an EGFP reporter gene (SEQ ID NO: 10) and a Rho-targeting spacer sequence (SEQ ID NO: 11) for additional testing.

### Transfection and Detection:

To evaluate the cleavage and collateral activities of Cas13f mutants in mammalian cells, the two plasmids were co-transfected into HEK293T cells. Expression levels of EGFP and mCherry were measured 72 hours by florescent measurement after the co-transfection. Low EGFP mean fluorescent intensity (MFI) indicated high cleavage activity as desired. High mCherry MFI indicated low or no collateral cleavage activity as desired.

According to standard cell culture methods, HEK293T cells were grown in 24-well tissue culture plates to a suitable density before the cells were transfected with both plasmids using a PEI transfection reagent. Transfected cells were cultured at 37° C. in an incubator under 5% CO₂ for about 72 hours, before measuring EGFP and mCherry fluorescent signals in the cells with FACS. Cas13f mutants leading to both low EGFP MFI and high mCherry MFI were selected.

All the MFI results (mean±SD) of the Cas13f mutants were normalized to the negative control.

RT-qPCR was carried out for an additional genome locus, SOD1, to investigate SOD1 mRNA knockdown indicative of cleavage activities of Cas13f mutants. According to standard cell culture methods, Cos7 cells were grown in 6-well tissue culture plates to a suitable density before the cells were transfected with the Cas13f mutant encoding plasmid (with SOD1-targeting spacer sequence of SEQ ID NO: 12) using a PEI transfection reagent. After 72 hours, an amount of the top 30% mCherry-positive cells were sorted by flow sorting, total RNA was extracted from the positive cells, and SOD1 mRNA level was measured by RT-qPCR and normalized to a housekeeping gene, GAPDH.

### Results:

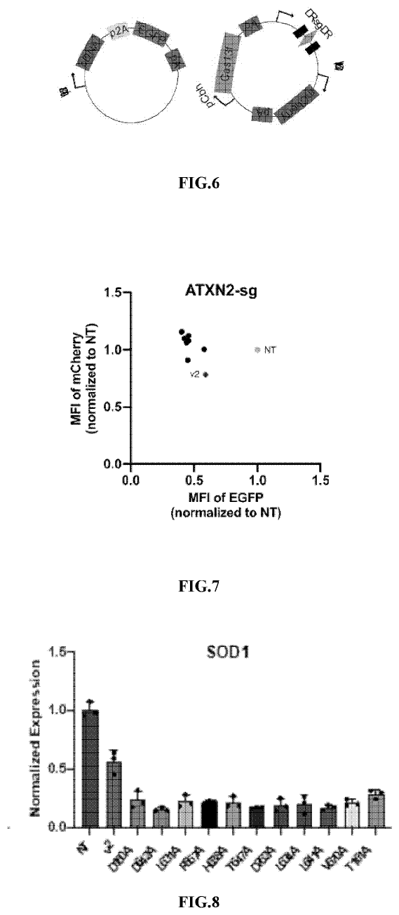

Cas13f mutants located at the upper left area of FIG. 7 have not only higher cleavage activity (low EGFP MFI) but also lower collateral cleavage activity (high mCherry MFI) (Table 7) than Cas13f v2. Among others, v2+L641A was designated as Cas13f v2.5.

TABLE 7
Averaged cleavage and collateral activities of Cas13f mutants,
as presented by MFIs with gRNA targeting ATXN2 RNA transcript
(spacer sequence, SEQ ID NO: 8) (n = 3)

| | MFI of | MFI of |
|---|---|---|
| Mutant | mCherry | EGFP |
| NT | 1.000 | 1.000 |
| v2 | 0.781 | 0.590 |
| v2 + D160A | 0.908 | 0.449 |
| v2 + P667A | 1.060 | 0.440 |
| v2 + T647A | 1.122 | 0.456 |
| v2 + D762A | 1.156 | 0.403 |
| v2 + L641A | 1.097 | 0.424 |
| v2 + A763V | 1.003 | 0.579 |
| v2 + T161A | 1.078 | 0.454 |

The RT-qPCR results show the improved SOD1 mRNA knockdown efficiency of the indicated Cas13f mutants than Cas13f v2 (FIG. 8, Table 8).

TABLE 8
Averaged SOD1 mRNA level in Cos7 cells by RT-qPCR for Cas13f
mutants, n = 3 (spacer SEQ ID NO: 12) (n = 3)

| | Averaged SOD1 |
|---|---|
| Mutant | mRNA level |
| NT | 1.001 |
| v2 | 0.562 |
| v2 + D160A | 0.233 |
| v2 + D642A | 0.153 |
| v2 + L631A | 0.221 |
| v2 + P667A | 0.218 |
| v2 + H638A | 0.208 |
| v2 + T647A | 0.166 |
| v2 + D762A | 0.189 |
| v2 + L634A | 0.197 |
| v2 + L641A | 0.171 |
| v2 + V670A | 0.208 |
| v2 + T161A | 0.285 |

The above results show that the additional introduction of a single-point mutation listed in Table 6 into Cas13f v2 enhanced the cleavage activity while maintaining or even lowering the collateral activity of Cas13f v2.

Based on the above results and with the same experimental procedures, the single mutations were subsequently combined in pair for introduction into Cas13f v2. Among others, Cas13f v2+D160A&D642A was designated as Cas13f v3 (SEQ ID NO: 4).

TABLE 9
Averaged cleavage and collateral activities of Cas13f mutants
as presented by MFI with gRNA targeting Rho RNA transcript
(spacer sequence, SEQ ID NO: 11) (n = 3) (FIG. 9)

| | MFI of | MFI of |
|---|---|---|
| Mutant | mCherry | EGFP |
| NT | 1.000 | 1.000 |
| v2 | 0.869 | 0.665 |
| v2 + D160A | 0.892 | 0.578 |
| v2 + D642A | 1.084 | 0.497 |
| v2 + L631A | 0.921 | 0.533 |
| v2 + P667A | 0.964 | 0.528 |
| v2 + H638A | 0.913 | 0.540 |
| v2 + L634A | 0.956 | 0.620 |
| v2 + L641A | 1.058 | 0.636 |
| v2 + L631A&H638A | 0.978 | 0.640 |
| v2 + L631A&L641A | 1.055 | 0.840 |
| v2 + L631A&D642A | 0.966 | 0.655 |
| v2 + D160A&L631A | 0.968 | 0.469 |
| v2 + H638A&L641A | 0.909 | 0.700 |
| v2 + H638A&D642A | 0.921 | 0.464 |
| v2 + L641A&D642A | 0.995 | 0.551 |
| v2 + D160A&D642A (Cas13f v3) | 1.113 | 0.430 |

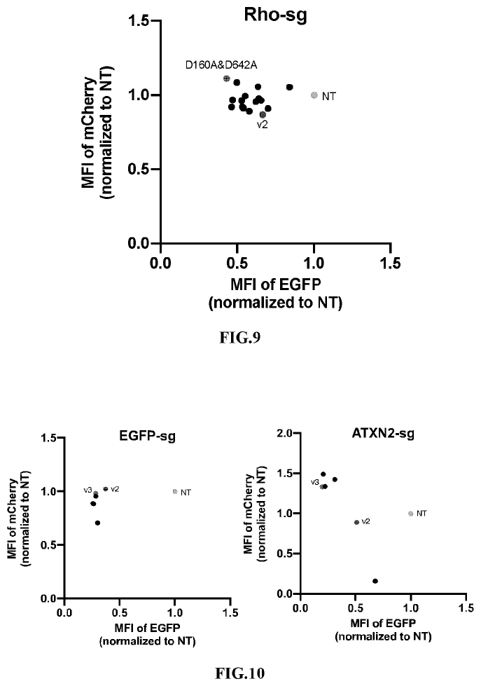

TABLE 10
Averaged cleavage and collateral cleavage activities of Cas13f mutants
as presented by MFIs with gRNA targeting EGFP RNA transcript (spacer
sequence, SEQ ID NO: 6) (n = 3) (FIG. 10, left panel)

| | MFI of | MFI of |
|---|---|---|
| Mutant | mCherry | EGFP |
| NT | 1.000 | 1.000 |
| v2 | 1.024 | 0.374 |
| v2 + D160A | 0.709 | 0.301 |
| v2 + H638A | 0.889 | 0.259 |
| v2 + D642A | 0.885 | 0.265 |
| v3 | 0.982 | 0.283 |
| v2 + H638A&D642A | 0.957 | 0.284 |

TABLE 11
Averaged cleavage and collateral cleavage activities
of Cas13f mutants as presented by MFIs with gRNA
targeting ATXN2 RNA transcript (spacer sequence,
SEQ ID NO: 8) (n = 3) (FIG. 10, right panel)

| | MFI of | MFI of |
|---|---|---|
| Mutant | mCherry | EGFP |
| NT | 1.000 | 1.000 |
| v2 | 0.891 | 0.510 |
| v2 + D160A | 1.492 | 0.209 |
| v2 + H638A | 0.161 | 0.679 |
| v2 + D642A | 1.425 | 0.313 |
| v3 | 1.335 | 0.202 |
| v2 + H638A&D642A | 1.338 | 0.225 |

TABLE 12
Averaged SOD1 mRNA level in Cos7 cells by RT-qPCR
for Cas13f mutants, n = 3 (spacer sequence,
SEQ ID NO: 12) (n = 3) (FIG. 11)

| | Averaged SOD1 |
|---|---|
| Protein | mRNA level |
| NT | 1.005 |
| v2 | 0.307 |
| v3 | 0.125 |
| v2 + H638A&D642A | 0.202 |

Among others, both the flow cytometry results (FIG. 9-10, Tables 9-11) and RT-qPCR results (FIG. 11, Table 12) show both the higher cleavage activity and lower collateral activity of Cas13f v3 than Cas13f v2.

### Example 3 Further Engineering of Cas13f for Increased Cleavage Activity

This Example demonstrates that by introducing a specific amino acid mutation, the cleavage activity of Cas13f v3 can be increased.

### Designs and Constructions:

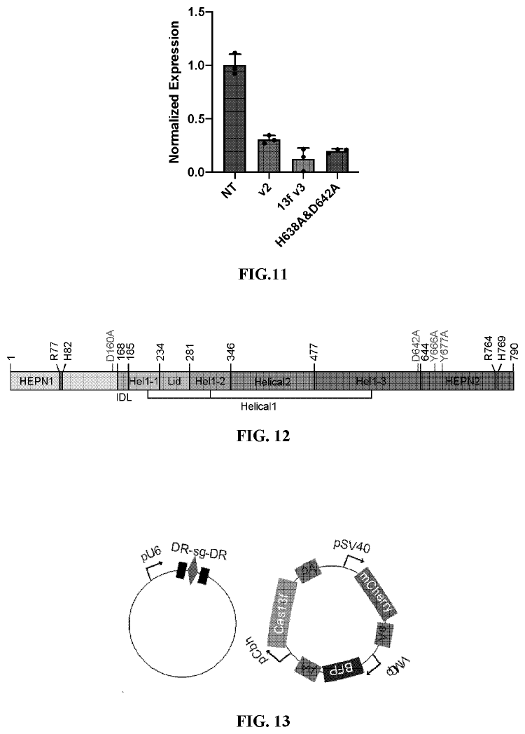

RNA is a negatively charged molecule that prefers to interact with positively charged basic amino acids in protein. To obtain a Cas13f mutant with increased cleavage activity, one of the non-basic amino acids of Cas13f v3 protein except those in the HEPN1 and HEPN2 domains was mutated to arginine (R, a common positively charged basic amino acid) to create a Cas13f mutant based on Cas13f v3 (FIG. 12). A two-plasmid mammalian fluorescence reporter system was constructed for detection of the cleavage activities of Cas13f mutants as shown in FIG. 13.

One plasmid comprised a red fluorescent reporter gene (mCherry) under the regulation of a SV40 promoter and a poly A sequence, a Cas13f mutant coding sequence flanked by both 5′ and 3′ terminal SV40 NLS (SEQ ID NO: 5) coding sequence under the regulation of a Cbh promoter and a poly A sequence, and a BFP fluorescent reporter gene under the regulation of a CMV promoter and a poly A sequence. The blue fluorescence from BFP would indicate successful transfection and expression of the plasmid in host cells.

The other plasmid comprised a sequence encoding a gRNA in 5′-DR sequence (SEQ ID NO: 2) -mCherry-targeting spacer sequence (SEQ ID NO: 13)-DR sequence (SEQ ID NO: 2)-3′ configuration under the regulation of a U6 promoter. As a negative control, a non-targeting spacer sequence (“NT”, SEQ ID NO: 14) was used in place of the mCherry-targeting spacer sequence (SEQ ID NO: 13) in the plasmid.

### Transfection and Detection:

HEK293T cells were cultured in 24-well tissue culture plates according to standard methods for 12 hours, before the two plasmids were co-transfected into the cells using standard polyethyleneimine (PEI) transfection. The transfected cells were then cultured at 37° C. under 5% CO₂ for about 48 hours. Then the cultured cells were analyzed by flow cytometry. The cleavage activity of each Cas13f mutant was calculated as the mean red fluorescence intensity (“RFP MFI”, weaker RFP MFI indicating higher cleavage activity) of BFP positive cells (“BFP⁺”, indicating successful transfection and expression of the plasmid).

### Results:

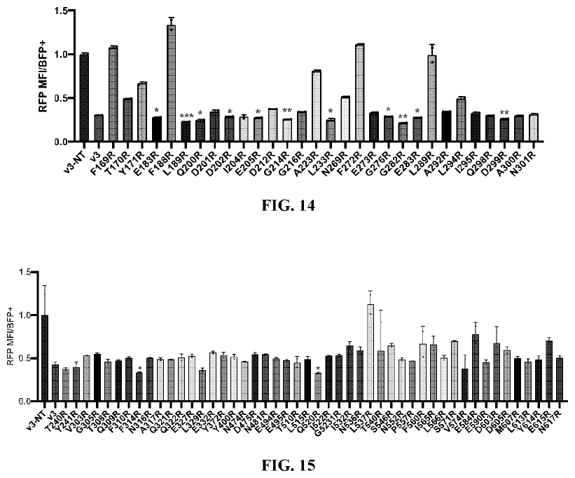

The Cas13f mutants were tested in batches with Cas13f v3, thereby excluding the effect of transfection efficiency on cleavage activity. The flow cytometry results show the RFP MFI of each Cas13f mutant with a single amino acid substitution to R. Among others, the Cas13f mutants with a single amino acid substitution to Rat position 183, 189, 200, 202, 205, 214, 233, 276, 282, 283, 299, 314, 520, 258, 259, 339, 410, 433, 595, 598, 213, 338, 508, or 526 on the basis of Cas13f v3 had weaker RFP MFI than that Cas13fv3, indicating increased cleavage activities (Table. 13-16 and FIGS. 14-17).

TABLE 13
Averaged RFP MFI (n = 2) of BFP
positive cells for Cas13f mutants (FIG. 14)

| | Averaged | | Averaged | | Averaged |
|---|---|---|---|---|---|
| Mutant | RFP MFI | Mutant | RFP MFI | Mutant | RFP MFI |
| V3-NT | 1.000 | I204R | 0.286 | G282R | 0.212 |
| V3 | 0.304 | E205R | 0.273 | E283R | 0.276 |
| F169R | 1.079 | D212R | 0.376 | L289R | 0.994 |
| T170R | 0.490 | G214R | 0.257 | A292R | 0.346 |
| Y171R | 0.668 | G216R | 0.343 | L294R | 0.495 |
| E183R | 0.276 | A223R | 0.807 | I295R | 0.327 |
| F188R | 1.339 | L233R | 0.252 | Q298R | 0.298 |
| L189R | 0.231 | N269R | 0.511 | D299R | 0.263 |
| Q200R | 0.246 | F272R | 1.110 | A300R | 0.295 |
| D201R | 0.346 | E273R | 0.330 | N301R | 0.311 |
| D202R | 0.286 | G276R | 0.289 | | |

TABLE 14
Averaged RFP MFI (n = 2 or 1) of BFP
positive cells for Cas13f mutants (FIG. 15)

| | Averaged | | Averaged | | Averaged |
|---|---|---|---|---|---|
| Mutant | RFP MFI | Mutant | RFP MFI | Mutant | RFP MFI |
| V3-NT | 1.000 | Y372R | 0.534 | N552R | 0.486 |
| V3 | 0.428 | Y400R | 0.515 | P557R | 0.469 |
| T240R | 0.374 | N474R | 0.460 | F560R | 0.667 |
| Y241R | 0.395 | D475R | 0.544 | I565R | 0.659 |
| V303R | 0.534 | N481R | 0.544 | L566R | 0.504 |
| G305R | 0.549 | E494R | 0.499 | S571R | 0.699 |
| T308R | 0.457 | E495R | 0.477 | V574R | 0.379 |
| Q309R | 0.472 | T510R | 0.447 | E584R | 0.774 |
| F310R | 0.504 | L515R | 0.491 | E590R | 0.453 |
| F314R | 0.336 | Q520R | 0.330 | D603R | 0.677 |
| N316R | 0.504 | I522R | 0.527 | D605R | 0.594 |
| A317R | 0.489 | G5231R | 0.531 | M607R | 0.501 |
| Q321R | 0.489 | I532R | 0.648 | L613R | 0.459 |
| Q322R | 0.509 | N536R | 0.590 | Y614R | 0.487 |
| E327R | 0.525 | L537R | 1.127 | E615R | 0.707 |
| L329R | 0.364 | T540R | 0.586 | N617R | 0.506 |
| E332R | 0.565 | S546R | 0.647 | | |

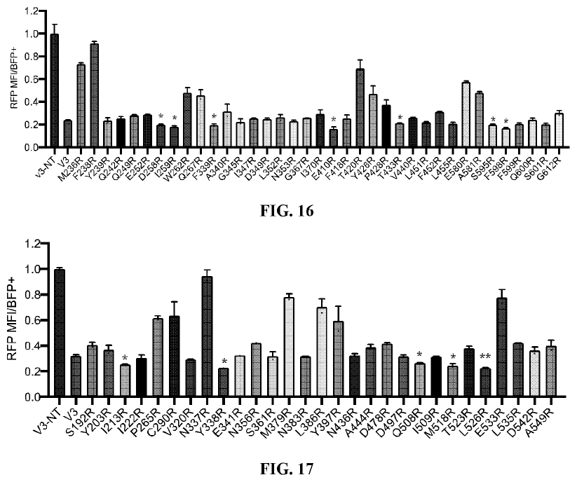

TABLE 15
Averaged RFP MFI (n = 2) of BFP
positive cells for Cas13f mutants (FIG. 16)

| | Averaged | | Averaged | | Averaged |
|---|---|---|---|---|---|
| Mutant | RFP MFI | Mutant | RFP MFI | Mutant | RFP MFI |
| V3-NT | 1.000 | A340R | 0.316 | T433R | 0.212 |
| V3 | 0.238 | G345R | 0.225 | V440R | 0.260 |
| M236R | 0.731 | I347R | 0.253 | L451R | 0.221 |
| F238R | 0.915 | D349R | 0.246 | F452R | 0.312 |
| Y239R | 0.237 | L352R | 0.260 | L455R | 0.206 |
| Q242R | 0.253 | N353R | 0.230 | E580R | 0.575 |
| Q249R | 0.282 | G367R | 0.257 | A581R | 0.479 |
| E252R | 0.289 | I370R | 0.294 | S595R | 0.198 |
| D258R | 0.199 | E410R | 0.163 | F598R | 0.168 |
| I259R | 0.182 | F418R | 0.252 | F599R | 0.202 |
| W262R | 0.479 | T420R | 0.693 | Q600R | 0.241 |
| Q267R | 0.458 | Y426R | 0.471 | S601R | 0.201 |
| F339R | 0.195 | P428R | 0.374 | G612R | 0.301 |

TABLE 16
Averaged RFP MFI (n = 2 or 1) of BFP
positive cells for Cas13f mutants (FIG. 17)

| | Averaged | | Averaged | | Averaged |
|---|---|---|---|---|---|
| Mutant | RFP MFI | Mutant | RFP MFI | Mutant | RFP MFI |
| V3-NT | 1.000 | E341R | 0.325 | Q508R | 0.263 |
| V3 | 0.320 | N356R | 0.422 | I509R | 0.313 |
| S192R | 0.406 | S361R | 0.316 | M518R | 0.245 |
| Y203R | 0.369 | M379R | 0.782 | T523R | 0.381 |
| I213R | 0.253 | N383R | 0.316 | L526R | 0.226 |
| I222R | 0.303 | L386R | 0.703 | E533R | 0.776 |
| P265R | 0.617 | Y397R | 0.596 | L535R | 0.421 |
| C290R | 0.635 | N436R | 0.324 | D542R | 0.363 |
| V320R | 0.294 | A444R | 0.389 | A549R | 0.399 |
| N337R | 0.945 | D478R | 0.416 | | |
| Y338R | 0.227 | D497R | 0.317 | | |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An engineered Cas13f polypeptide, wherein the engineered Cas13f polypeptide:
(1) comprises a mutation in a region spatially close to a) the N-terminal endonuclease catalytic RXXXXH motif (e.g., the N-terminal endonuclease catalytic RNFYSH motif) of a reference Cas13f polypeptide (e.g., of SEQ ID NO: 1), and/or b) the C-terminal endonuclease catalytic RXXXXH motif (e.g., the C-terminal endonuclease catalytic RNKALH motif) of the reference Cas13f polypeptide (e.g., of SEQ ID NO: 1);
(2) substantially preserves (e.g., having at least about 50%, 60%, 70%, 72.5%, 75%, 77.5%, 80%, 82.5%, 85%, 87.5%, 90%, 92.5%, 95%, 96%, 97%, 98%, 99%, or more of) the spacer sequence-specific cleavage activity of the reference Cas13f polypeptide (e.g., of SEQ ID NO: 1) towards a target RNA complementary to the spacer sequence; and
(3) substantially lacks (e.g., having no more than about 50%, 45%, 40%, 35%, 30%, 27.5%, 25%, 22.5%, 20%, 17.5%, 15%, 12.5%, 10%, 7.5% 5%, 4.5%, 4%, 3.5%, 3%, 2.5%, 2%, 1.5%, 1% or less of) the spacer sequence-independent collateral cleavage activity of the reference Cas13f polypeptide (e.g., of SEQ ID NO: 1) towards a non-target RNA that does not bind to the spacer sequence.

2. The engineered Cas13f polypeptide of claim 1, wherein the region includes residues within 140, 130, 120, 110, 100, 90, 80, 70, 60, 50, 40, 30, 20, or 10 amino acids from any residues of the N-terminal endonuclease catalytic RXXXXH motif or the C-terminal endonuclease catalytic RXXXXH motif; or
wherein the region includes residues more than 100, 110, 120, or 130 residues away from any residues of the N-terminal endonuclease catalytic RXXXXH motif or the C-terminal endonuclease catalytic RXXXXH motif but are spatially within about 1 to about 10 or about 5 Angstrom of any residue of the N-terminal endonuclease catalytic RXXXXH motif or the C-terminal endonuclease catalytic RXXXXH motif.

3. (canceled)

4. The engineered Cas13f polypeptide of claim 1, wherein the region comprises, consists essentially of, or consists of residues corresponding to the HEPN1 domain (e.g., residues 1-168), the IDL domain (e.g., residues 168-185), the Helical1 domain (e.g., Helical1-1 (Hell-1) domain (e.g., residues 185-234), Helical1-2 (Hell-2) domain (e.g., residues 281-346), Helical1-3 (Hell-3) domain (e.g., residues 477-644)), the Helical2 domain (e.g., residues 346-477), or the HEPN2 domain (e.g., residues 644-790) of the reference Cas13f polypeptide of SEQ ID NO: 1.

5. The engineered Cas13f polypeptide of claim 1, wherein the mutation comprises, consists essentially of, or consists of, within a stretch of about 8 to about 20 (e.g., about 9 or about 17) consecutive amino acids within the region,
(a) substitution(s) of one or more (e.g., 1, 2, 3, 4, 5, or more) non-Ala (A) residues to Ala (A) residues;
(b) substitution(s) of one or more (e.g., 1, 2, 3, 4, 5, or more) charged residues, nitrogen-containing side chain group residues, bulky (such as F or Y) residues, aliphatic residues, and/or polar residues to charge-neutral short chain aliphatic residues (such as A, V, or I);
(c) substitution(s) of one or more (e.g., 1, 2, 3, 4, 5, or more) Ile (I) and/or Leu (L) residues to Ala (A) residues; and/or
(d) substitution(s) of one or more (e.g., 1, 2, 3, 4, 5, or more) Ala (A) residues to Val (V) residues;
optionally wherein the one or more non-Ala residues and/or the one or more charged or polar residues comprise N, Q, R, K, H, D, E, Y, S, T, L residues or a combination thereof;
and/or wherein the one or more non-Ala residues and/or the one or more charged or polar residues comprise N, Q, R, K, H, D, Y, L residues or a combination thereof.

6-7. (canceled)

8. The engineered Cas13f polypeptide of claim 5, wherein one or more Y residue(s) within the stretch is substituted: wherein the one or more Y residues(s) correspond to Y666 and/or Y677 of the reference Cas13f polypeptide of SEQ ID NO: 1; and/or
wherein the engineered Cas13f polypeptide has one or more D residue(s) within the stretch is substituted: wherein the one or more D residues(s) correspond to D160 and/or D642 of the reference Cas13f polypeptide of SEQ ID NO: 1; and/or
wherein the engineered Cas13f polypeptide has charge-neutral short chain aliphatic residue that is Ala (A).

9-12. (canceled)

13. The engineered Cas13f polypeptide of claim 1, wherein the mutation comprises, consists essentially of, or consists of:
(a) substitutions within 1, 2, 3, 4, or 5 of the stretches of about 8 to about 20 (e.g., about 9 or about 17) consecutive amino acids within the region;
(b) a mutation corresponding to a mutation (e.g., any one in Tables 1-5) that results in an engineered Cas13f polypeptide having at least about 75% of a spacer sequence-specific cleavage activity and no more than about 25% of a spacer sequence-independent collateral cleavage activity, or a combination thereof; and/or
(c) a mutation corresponding to the F7V2, F10V1, F10V4, F40V4, F40S22, F40S26, F40S36, F10S21, F10S24, F10S26, F10S27, F10S33, F10S34, F10S35, F10S36, F10S45, F10S46, F10S48, F10S49, F40S23, or F40S27 mutation in Table 5, or a combination thereof.

14. The engineered Cas13f polypeptide of claim 1, wherein the engineered Cas13f polypeptide retains at least about 50%, 60%, 70%, 72.5%, 75%, 77.5%, 80%, 82.5%, 85%, 87.5%, 90%, 92.5%, 95%, 96%, 97%, 98%, 99%, or more of the spacer sequence-specific cleavage activity of the reference Cas13f polypeptide of SEQ ID NO: 1 towards the target RNA;
wherein the engineered Cas13f polypeptide has no more than 50%, 45%, 40%, 35%, 30%, 27.5%, 25%, 22.5%, 20%, 17.5%, 15%, 12.5%, 10%, 7.5%, 5%, 4.5%, 4%, 3.5%, 3%, 2.5%, 2%, 1.5%, 1%, or less of the spacer sequence-independent collateral cleavage activity of the reference Cas13f polypeptide of SEQ ID NO: 1 towards the non-target RNA; and/or
wherein the engineered Cas13f polypeptide has at least about 80% of the spacer sequence-specific cleavage activity of the reference Cas13f polypeptide of SEQ ID NO: 1 towards the target RNA and no more than about 40% of the spacer sequence-independent collateral cleavage activity of the reference Cas13f polypeptide of SEQ ID NO: 1 towards the non-target RNA.

15. The engineered Cas13f polypeptide of claim 14, wherein the mutation is F40S23 (i.e., Y666A/Y677A double mutation); and/or
wherein the engineered Cas13f polypeptide, comprising, consisting essentially of, or consisting of the amino acid sequence of SEQ ID NO: 3.

16. (canceled)

17. The engineered Cas13f polypeptide of claim 1, further comprising a mutation corresponding to a combination of any one, two, or more (e.g., 3, 4, or 5 more) mutations in Table 6 (such as, D160A, D642A, and/or L641A); and/or
wherein the mutation is a combination of any one, two, or more (e.g., 3, 4, or 5 more) single mutations in Table 6 (such as, D160A, D642A, and/or L641A) with F40S23 (i.e., Y666A/Y677A double mutation); and/or
wherein the mutation is a Y666A/Y677A double mutation in combination with 1, 2, or 3 mutations selected from D160A, L641A, and D642A.

18-19. (canceled)

20. The engineered Cas13f polypeptide of claim 1, wherein the mutation is any combination mutations in Tables 7-12;
optionally wherein the mutation is a D160A/D642A/Y666A/Y677A quadruple mutation.

21. (canceled)

22. The engineered Cas13f polypeptide of claim 1, wherein the engineered Cas13f polypeptide has increased spacer sequence-specific cleavage activity than that of the engineered Cas13f polypeptide of SEQ ID NO: 3; and/or
wherein the engineered Cas13f polypeptide has a mutation corresponding to a combination of a mutation in Tables 13-16 with D160A/D642A/Y666A/Y677A mutation;
and/or wherein the engineered Cas13f polypeptide comprises, consists essentially of, or consists of the amino acid sequence of SEQ ID NO: 4; and/or
wherein the engineered Cas13f polypeptide further comprises an amino acid substitution of a non-basic amino acid residue to Arg (R) residue: optionally further comprises a mutation corresponding to a combination of any one, two, or more (e.g., 3, 4, or 5 more) single mutations in Tables 13-16.

23-25. (canceled)

26. The engineered Cas13f polypeptide of claim 1, wherein the engineered Cas13f polypeptide has increased spacer sequence-specific cleavage activity than that of the engineered Cas13f polypeptide of SEQ ID NO: 4; and/or
wherein the engineered Cas13f polypeptide has a sequence identity of at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% and less than 100% to the reference Cas13f polypeptide of SEQ ID NO: 1; and/or
wherein the engineered Cas13f polypeptide further comprises a nuclear localization signal (NLS) sequence or a nuclear export signal (NES); optionally comprising an N- and/or a C-terminal NLS.

27-28. (canceled)

29. A polynucleotide encoding the engineered Cas13f polypeptide of claim 1;
optionally the polynucleotide is codon-optimized for expression in a eukaryote, a mammal, such as a human or a non-human mammal, a plant, an insect, a bird, a reptile, a rodent (e.g., mouse, rat), a fish, a worm/nematode, or a yeast.

30. A CRISPR-Cas13f system comprising:
a) the engineered Cas13f polypeptide of claim 1 or a polynucleotide coding sequence (e.g., a DNA coding sequence or an RNA coding sequence) thereof, and
b) a guide RNA (gRNA) or a polynucleotide coding sequence (e.g., a DNA coding sequence or an RNA coding sequence) thereof, the gRNA comprising:
i. a direct repeat (DR) sequence capable of forming a complex with the engineered Cas13f polypeptide; and,
ii. a spacer sequence capable of hybridizing to a target RNA, and guiding or recruiting the complex to the target RNA;
optionally wherein the DR sequence has substantially the same secondary structure of that of SEQ ID NO: 2; and
optionally wherein the spacer sequence is in a length of at least 15 nucleotides, optionally 30 nucleotides.

31. A vector comprising the polynucleotide of claim 29;
optionally wherein the polynucleotide is operably linked to a promoter and optionally an enhancer;
optionally wherein the promoter is a constitutive promoter, an inducible promoter, a ubiquitous promoter, or a cell, tissue, or organ specific promoter;
optionally wherein the vector is a plasmid;
optionally wherein the vector is a retroviral vector, a phage vector, an adenoviral vector, a herpes simplex viral (HSV) vector, an AAV vector, or a lentiviral vector;
optionally wherein the AAV vector is a recombinant AAV vector of the serotype AAV1, AAV2, AAV4, AAV5, AAV6, AAV7, AAVrh74, AAV8, AAV9, AAV10, AAV 11, AAV 12, AAV 13, AAV.PHP.eB, or AAV-DJ; and/or
optionally wherein the AAV vector is an RNA-encapsulated AAV vector.

32. A delivery system comprising (1) a delivery vehicle, and (2) the engineered Cas13f polypeptide of claim 1;
optionally wherein the delivery vehicle is a nanoparticle (e.g., LNP), a liposome, an exosome, a microvesicle, or a gene-gun.

33. A cell or a progeny thereof, comprising the engineered Cas13f polypeptide of claim 1;
optionally wherein the cell is a eukaryotic cell (e.g., a non-human mammalian cell, a human cell, or a plant cell) or a prokaryotic cell (e.g., a bacteria cell).

34. A non-human multicellular eukaryote comprising the cell or progeny of claim 33; optionally wherein the non-human multicellular eukaryote is an animal (e.g., rodent or primate) model for a human genetic disorder.

35. A method of modifying a target RNA, the method comprising contacting the target RNA with the CRISPR-Cas13f system of claim 30.

36-46. (canceled)

47. A method of treating a condition or disease in a subject in need thereof, the method comprising administering to the subject a composition comprising the CRISPR-Cas13f system of claim 30, wherein upon administrating, the engineered Cas13f polypeptide cleaves the target RNA, thereby treating the condition or disease in the subject.

48-51. (canceled)

52. A CRISPR-Cas13f complex comprising the engineered Cas13f polypeptide of claim 1, and a guide RNA comprising a DR sequence that binds the engineered Cas13f polypeptide and a spacer sequence capable of hybridizing to a target RNA, and guiding or recruiting the complex to the target RNA;
optionally wherein the target RNA is encoded by a eukaryotic DNA;
optionally wherein the eukaryotic DNA is a non-human mammalian DNA, a non-human primate DNA, a human DNA, a plant DNA, an insect DNA, a bird DNA, a reptile DNA, a rodent DNA, a fish DNA, a worm/nematode DNA, or a yeast DNA;
optionally wherein the target RNA is an mRNA; and/or
optionally wherein the CRISPR-Cas13f complex further comprises a target RNA comprising a sequence capable of hybridizing to the spacer sequence.


SEQUENCE LISTING (14 sequences)

SEQ ID NO: 1 - protein, 790 aa, unidentified
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNYPSLVYTMSSKYVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 2 - RNA, 36 nt, unidentified
  source 1..36: mol_type = other RNA
gctgtgatagacctcgatttgtggggtagtaacagc

SEQ ID NO: 3 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDDTGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLDDITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 4 - protein, 790 aa, synthetic construct
MNGIELKKEEAAFYFNQAELNLKAIEDNIFDKERRKTLLNNPQILAKMENFIFNFRDVTKNAKGEIDCLLLKLRELRNFYSHYVHKRDVRELSKGEKPILEKYYQFAIESTGSENVKLEIIENDAWLADAGVLFFLCIFLKKSQANKLISGISGFKRNDATGQPRRNLFTYFSIREGYKVVPEMQKHFLLFSLVNHLSNQDDYIEKAHQPYDIGEGLFFHRIASTFLNISGILRNMKFYTYQSKRLVEQRGELKREKDIFAWEEPFQGNSYFEINGHKGVIGEDELKELCYAFLIGNQDANKVEGRITQFLEKFRNANSVQQVKDDEMLKPEYFPANYFAESGVGRIKDRVLNRLNKAIKSNKAKKGEIIAYDKMREVMAFINNSLPVDEKLKPKDYKRYLGMVRFWDREKDNIKREFETKEWSKYLPSNFWTAKNLERVYGLAREKNAELFNKLKADVEKMDERELEKYQKINDAKDLANLRRLASDFGVKWEEKDWDEYSGQIKKQITDSQKLTIMKQRITAGLKKKHGIENLNLRITIDINKSRKAVLNRIAIPRGFVKRHILGWQESEKVSKKIREAECEILLSKEYEELSKQFFQSKDYDKMTRINGLYEKNKLIALMAVYLMGQLRILFKEHTKLADITKTTVDFKISDKVTVKIPFSNAPSLVYTMSSKAVDNIGNYGFSNKDKDKPILGKIDVIEKQRMEFIKEVLGFEKYLFDDKIIDKSKFADTATHISFAEIVEELVEKGWDKDRLTKLKDARNKALHGEILTGTSFDETKSLINELKK

SEQ ID NO: 5 - protein, 7 aa, Betapolyomavirus macacae
PKKKRKV

SEQ ID NO: 6 - RNA, 30 nt, synthetic construct
  source 1..30: mol_type = other RNA
gtcctccttgaagtcgatgcccttcagctc

SEQ ID NO: 7 - DNA, 1041 bp, synthetic construct
  source 1..1041: mol_type = other DNA
atgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactgtcgacgccactaacttctccctgttgaaacaagcaggggatgtcgaagagaatcccgggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 8 - RNA, 30 nt, synthetic construct
  source 1..30: mol_type = other RNA
gtagagacaggagctggagaaccatgagca

SEQ ID NO: 9 - RNA, 23 nt, synthetic construct
  source 1..23: mol_type = other RNA
gagacccagatctgccggtctct

SEQ ID NO: 10 - DNA, 1206 bp, synthetic construct
  source 1..1206: mol_type = other DNA
atgaatggcacagaaggccctaacttctacgtgcccttctccaatgcgacgggtgtggtacgcagccacttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccgtcgacgccactaacttctccctgttgaaacaagcaggggatgtcgaagagaatcccgggccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 11 - RNA, 30 nt, synthetic construct
  source 1..30: mol_type = other RNA
gaagccacctaggaccatgaagaggtcagc

SEQ ID NO: 12 - RNA, 30 nt, synthetic construct
  source 1..30: mol_type = other RNA
gcctctcttcatcctttggcccaccgtgtt

SEQ ID NO: 13 - RNA, 30 nt, synthetic construct
  source 1..30: mol_type = other RNA
gcagcttcaccttgtagatgaactcgccgt

SEQ ID NO: 14 - RNA, 30 nt, synthetic construct
  source 1..30: mol_type = other RNA
ggtcttcgatattcaagcgtcggaagacct